(19) United States
(12) Patent Application Publication
(10) Pub. No.: US 2010/0081679 A1
(43) Pub. Date: Apr. 1, 2010

(54) DIAMINOPYRIMIDINES AS FUNGICIDES

(75) Inventors: Joerg Nico Greul, Leichlingen (DE); Oliver Gaertzen, Koeln (DE); Ralf Dunkel, Leichlingen (DE); Amos Mattes, Langenfeld (DE); Stefen Hillebrand, Neuss (DE); Ulrike Wachendorff-Neumann, Neuwied (DE); Peter Dahmen, Neuss (DE); Arnd Voerste, Koeln (DE); Peter Schreier, Koeln (DE); Pierre-Yves Coqueron, Lyon (FR)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim (DE)

(21) Appl. No.: 12/529,612

(22) PCT Filed: Feb. 26, 2008
(86) PCT No.: PCT/EP2008/001503

§ 371 Date: Nov. 4, 2009

(30) Foreign Application Priority Data
Mar. 2, 2007 (DE) 10 2007 010 801.1

(51) Int. Cl. A01N 43/54; C07D 239/48; C07D 401/12; A01P 3/00

(52) U.S. Cl. 514/275; 544/323; 544/324

(57) ABSTRACT

Use of diaminopyrimidines of the formula (I)

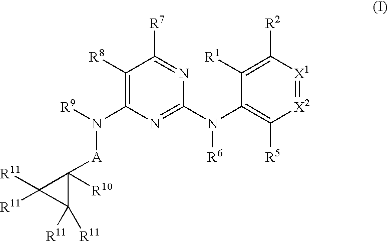

in which
R¹, R², R³, R⁴, R⁵, R⁶, R⁷, R⁸, R⁹, R¹⁰, R¹¹ and X¹, X² and A have the meanings given in the description, and also agrochemically active salts thereof as fungicides.

Diaminopyrimidines of the formulae (Ia), (Ib), (Ic), (Id), (Ie), (If)

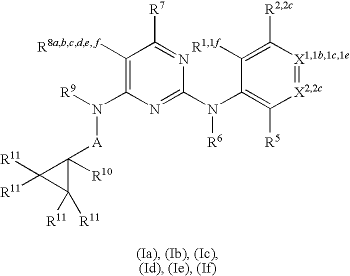

in which R^{8a}, R^{8b}, R^{8c}, R^{8d}, R^{8e}, R^{8f}, R^{3b}, R^{3c}, R^{3e}, X^{1b}, X^{1c}, X^{1e} and R¹, R², R³, R⁴, R⁵, R⁶, R⁷, R⁸, R⁹, R¹⁰, R¹¹ and X¹, X² and A have the meanings given in the description, and also agrochemically active salts thereof and their use for controlling unwanted microorganisms.

The invention relates to cyclopropyl-substituted diaminopyrimidines and their use for controlling unwanted microorganisms.

It is already known that certain alkynyl-substituted diaminopyrimidines can be used as fungicidal crop protection agents (see DE 4029650 A1). However, in particular at low application rates, the fungicidal activity of these compounds is not always sufficient.

Since the ecological and economical demands made on modern fungicides are increasing constantly, for example with respect to activity spectrum, toxicity, selectivity, application rate, formation of residues and favourable manufacture, and there can furthermore be problems, for example, with resistance, there is a constant need to develop novel fungicides which, at least in some areas, have advantages over those of the prior art.

Surprisingly, it has now been found that the present cyclopropyl-substituted diaminopyrimidines achieve the objects mentioned at least in some aspects and are therefore suitable as fungicides.

Some of these cyclopropyl-substituted diaminopyrimidines are already known as pharmaceutically active compounds (see, for example, US 04/256145, WO 05/016893, WO 05/013996, WO 04/056786, WO 04/056807, WO 03/076437, WO 02/096888, WO 02/004429), but not their surprising fungicidal activity.

The invention provides the use of compounds of the formula (I) as fungicides,

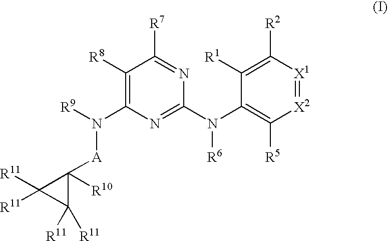

where the symbols have the following meanings:

X¹ represents nitrogen or CR³,

X² represents nitrogen or CR⁴,

A represents C(R¹⁴)₂ or a direct bond,

R¹ to R⁵ independently of one another represent hydrogen, halogen, cyano, hydroxyl, nitro, a 3- to 8-membered unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹²; (CH₂)ₘSR¹², (CH₂)ₘSOR¹², (CH₂)ₘSO₂R¹², (CH₂)ₘSON(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl; where m=1-8,

where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,

R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)-carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,

R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,

R⁷ represents hydrogen, cyano, methyl, CF₃, CFH₂ or CF₂H,

R⁸ represents fluorine, chlorine, bromine, iodine, cyano, methyl, C₁-C₃-haloalkyl,

R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,

R¹⁰ represents hydrogen, C₁-C₈-alkyloxy, unsubstituted or substituted C₁-C₈-alkyl, C₃-C₈-cyloalkyl, C₁-C₈-haloalkyl, C₁-C₄-trialkylsilyl, unsubstituted or substituted aryl, COOR¹², C═OR¹², OR¹²,

R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,

R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,

or

if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,

or

if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,

R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,

R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S),

and agrochemically active salts thereof as fungicides.

Compounds of the formula (I) are highly suitable for controlling unwanted microorganisms. In particular, they have strong fungicidal activity and can be used both in crop protection and in the protection of materials.

The compounds of the formula (I) can be present either in pure form or as mixtures of various possible isomeric forms, in particular of stereoisomers, such as, for example, E and Z, threo and erythro, and also optical isomers, such as R and S isomers or atropisomers, and, if appropriate, also of tautomers. What is claimed are both the E and the Z isomers and the threo and erythro and also the optical isomers, any mixtures of these isomers, and also the possible tautomeric forms.

The formula (I) provides a general description of the compounds according to the invention.

Preference is given to using, as fungicides, compounds of the formula (I) in which one or more of the symbols have one of the preferred meanings listed below, i.e.

X¹ represents nitrogen or CR³

X² represents nitrogen or CR⁴

A represents C(R¹⁴)₂ or a direct bond

R¹ to R⁵ independently of one another represent hydrogen, fluorine, chlorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF₃, OCH₂CF₂H_{,} OCH₂CF₂H_{,} OCH₂CF_{3,} OCH₂CF_{3,} OCH₂CH₂N(C₂H₅)_{2,} OCH₂CH₂N(CH₃)_{2,} OCH(CH₃)CH₂OCH_{3,} SH, SMe, SPh, SEt, SPr, SisoPr, SBu, SsecBu, SisoBu, StertBu, Spentyl, Ssecpentyl, Sneopentyl, S-Octyl, SCF₃, SCF₂H, SCFH₂, SOMe, SO-Et, SO—Pr, SOisoPr, SOBu, SOsecBu, SOisoBu, SOtertBu, SO-pentyl, SO₂Me, SO₂CF₃, SO₂Et, SO₂Pr, SO₂isoPr, SO₂Bu, SO₂secBu, SO₂isoBu, SO₂tertBu, SO₂-pentyl, SO₂CH₂CH═CH_{2,} SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtertBu, SONHpentyl, SONBu₂, SONHCF₃, SON(CF₃)₂, SO₂NHMe, SO₂NMe₂, SO₂NH_{2,} SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} COMe, COEt, COPr, COisoPr, COBu, COsecBu, COisoBu, COtertBu, COcyclopropyl, COCHF₂, COCH₂F, COCF₃, NHCOOMe, NHCOOisoPr, NHCOOtertBu, NHCOOnBu, NHCOOpentyl, NHCOOcycloproyl, NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(CH₃)COCH₃, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, NsecBu₂, NtetBu₂, NHMe, NH₂, NHtertBu, NHsecBu, NHEt, NHPr, NHiPr, NHBu, NHisoBu, NHsecBu, cyclopropylamino, NH(Et)cyclopropyl, NH(Et)cyclopropyl, NHCH(CH₃)CH₂OCH_{3,} NHCH(CH₃)CH₂OEt_{,} NCH₃COCH₃, NEtCOCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tertBu, NMeSOtertBu, NMeSO₂tertBu, NHSOsecBu, NHSO₂secBu, NMeSOsecBu, NMeSO₂secBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me)isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)_{2,} OSO₂NEt_{2,} CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tertBu, CO₂(CH₂)₂OH, CO₂(CH₂)₂OCH₃, CO₂(CH₂)₂OEt, COCH₂N(CH₃)₂, CO₂(CH₂)₃OCH₃, COCH₂NEt₂, CO₂(CH₂)₃OEt, CH₂SO₂NHMe, CH₂SO₂NHEt, CH₂SO₂NHisoPr, CH₂SO₂NHPr, CH₂SO₂NHtertBu, CH₂COtertBu, CH₂COCH₃, CH₂COEt, CH₂COOEt, CH₂COOMe, C(CH₃)₂OCH₃, CHCH₃OCH₃, CHCF₃OCH₃, CHCF₃OC₂H₅, C(CH₃)₂OEt, CHCH₃OEt, CHCF₃OEt, C(CH₃)₂OH, CHCH₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₂SH, (CH₂)₃SH, (CH₂)₄SH, CH₂SMe, (CH₂)₂SMe, (CH₂)₃SMe, (CH₂)₄SMe, CH₂SEt, (CH₂)₂SEt, (CH₂)₃SEt, (CH₂)₄SEt, CH₂NH₂, CH₂NAe₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe,(CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOispPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (I):

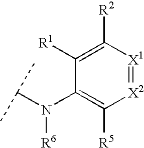

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino,

R⁶ represents hydrogen, benzyl, 2,4-dimethoxybenzyl, 4-methoxybenzyl, 2-hydroxybenzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkyl-sulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₄-trialkylsilyl or C₁-C₄-dialkylmonophenylsilyl, in which

R^{1-A} represents hydrogen, C₁-C₆-alkyl, C₁-C₆-haloalkyl or cyano,

R⁷ represents hydrogen, cyano, methyl, CF₃, CFH₂ or CF₂H,

R⁸ represents fluorine, chlorine, bromine, iodine, cyano, methyl, CF₃, CCl₃, CFH₂ or CF₂H,

R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, SO₂R¹³, SOR¹³,

R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,

R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl,

R¹² represents identical or different hydrogen, unsubstituted or substituted C₁-C₆-alkyl, unsubstituted or substituted C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₃-C₄-alkynyl, unsubstituted or substituted phenyl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another

or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,

or

if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,

R¹³ represents identical or different unsubstituted or substituted C₁-C₆-alkyl, unsubstituted or substituted C₁-C₆-haloalkyl, C₁-C₄-trialkylsilyl, unsubstituted or substituted C₁-C₄-alkenyl, unsubstituted or substituted C₁-C₄-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,

R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S),

and agrochemically active salts thereof as fungicides.

Particular preference is given to using, as fungicides, compounds of the formula (I) in which one or more of the symbols have one of the meanings below:

X¹ represents nitrogen or CR³,

X² represents nitrogen or CR⁴,

A represents C(R¹⁴)₂ or a direct bond,

R¹ to R⁵ independently of one another represent hydrogen, fluorine, chlorine, bromine, iodine, cyano, hydroxyl, nitro, OMe, OEt, OPr, OisoPr, OBu, OsecBu, OisoBu, OtertBu, O-(CH₂)₂OH, O—(CH₂)₂OCH₃, O—(CH₂)₃OH, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, SCF₃, SCF₂H, SOMe, SO-Et, SO—Pr, SOisoPr, SOBu, SOsecBu, SOisoBu, SO-tBu, Spentyl, Ssecpentyl, Sneopentyl, SOctyl, SO₂Me, SO₂CF₃, SO₂Et, SO₂Pr, SO₂isoPr, SO₂Bu, SO₂secBu, SO₂isoBu, SO₂-tBu, SO₂CH₂CH═CH_{2,} SO₂CH₂CN, SO₂CH₂C≡CH, SONHMe, SONMe₂, SONHEt, SONEt₂, SONHPr, SONPr₂, SONHnBu, SONBu₂, SONHCF_{3,} SON(CF₃)₂,═SO₂NHMe, SO₂NMe₂, SO₂NH_{2,} SO₂NHAc, SO₂NHPh, SO₂NHnBu, SO₂NHtBu, SO₂NEt₂, SO₂NHEt, SO₂NPr₂, SO₂NHPr, SO₂NHCF₃, SO₂N(CF₃)₂, SO₂NH(CH₂)₃NMe₂, SO₂NHCH₂CH═CH_{2,} COMe, COEt, COPr, COisoPr, COBu, COsecBu, COisoBu, COtertBu, COCHF₂, COCF₃, NHCOOMe, NHCOOisoPr, NHCOOtertBu, NHCOOnBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH_{3,} NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHsecBu, CONHisoBu, CONHtertBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONH(CH₂)₂OH, CONH(CH₂)₃OCH₃, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH,═CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, C(CH₃)₂OH, CHCH₃OH, CHCF₃OH, CH₂C(CH₃)₂OCH₃, CHCHF₂OCH₃, CHCHF₂OH, CH₂OH, CH₂NHCOOBn, CH₂NHCOOtertBu, CH₂SO₂CH₃, CH₂NH(CH₂)₂OCH₃, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)₂, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (I):

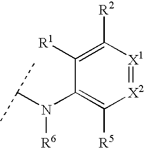

may be (2-oxo-2,3-dihydro-1H-indol-5-yl)aminoindol-5-yl)amino, 1H-indol-6-ylamino, 1H-indol-5-ylamino, 2-(trifluoromethyl)-1H-benzimidazol-6-yl]amino,(3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino,

R⁶ represents hydrogen, Me, CH₂OCH₃, formyl, COMe, COOMe, COOEt, COOtertBu, COOBn, COCF₃, benzyl, 2,4-dimethoxybenzyl, 4-methoxybenzyl, 2-hydroxybenzyl, C₂F₅OC(═O), CH₂CH═CH₂, CH₂C≡CH, SOCH₃, SO₂CH₃,

R⁷ represents hydrogen, cyano, methyl, CF₃, CFH₂ or CF₂H,

R⁸ represents fluorine, chlorine, bromine, iodine, cyano, methyl, CF₃, CCl₃, CFH₂ or CF₂H,

R⁹ represents hydrogen, Me, CH₂OCH₃, COMe, COOMe, COOEt, COOtertBu, COCF₃, benzyl, 4-methoxybenzyl, CH₂CH═CH₂, SO₂CH₃, CH₂C≡CH, SOCH₃,

R¹⁰ represents hydrogen, OH, OMe, OEt, OPr, Oi-Pr, OBu, OtBu, OiBu, OsBu, O-pentyl, Oneopentyl, Me, Et, Pr, i-Pr, Bu, tBu, iBu, sBu, pentyl, neopentyl, cyclopropyl, cyclobutyl, cyclopentyl, cyclohexyl, COOH, COOMe, COOEt, COOPr, COOi-Pr, COOBu, COOiBu, COOsBu, COOtBu, COO-pentyl, COOneopentyl, phenyl, 2-chlorophenyl, benzyl, COOMe, COEt, COMe, COPr, COisoPr, COBu, COtBu, COisoBu, COsBu, CO-pentyl, COneopentyl,

R¹¹ represents identical or different hydrogen, fluorine, chlorine, bromine,

R¹⁴ represents identical or different hydrogen, methyl, ethyl, cyclopropyl, cyclobutyl, cyclopentyl, isopropyl,
where two radicals R¹⁴ may also form a carbonyl or thiocarbonyl group (C═O or C═S),

and agrochemically active salts thereof as fungicides.

Very particular preference is given to using, as fungicides, compounds of the formula (I) in which one or more of the symbols have one of the meanings below:

X¹ represents nitrogen or CR³,

X² represents nitrogen or CR⁴,

A represents a direct bond, methylene or —CH(CH₃)— or —(C═O)—,

R¹ to R⁵ independently of one another represent hydrogen, fluorine, chlorine, bromine, iodine, cyano, hydroxyl, nitro, OMe, OEt, OPr, OisoPr, OBu, OsecBu, OisoBu, OtertBu, (CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBu, SOsecBu, SOisoBu, SO-tBu, SO₂Me, SO₂CF₃, SO₂Et, SO₂Pr, SO₂isoPr, SO₂Bu, SO₂secBu, SO₂isoBu, SO₂-tBu, SO₂CH₂CH═CH_{2,} SO₂CH₂CN, SO₂CH₂C≡CH, SONHMe, SONMe₂, SO₂NHMe, SO₂NMe₂, SO₂NH_{2,} SO₂NHAc, SO₂NHPh, SO₂NHnBu, SO₂NHtBu, SO₂NEt₂, SO₂NHEt, SO₂NPr₂, SO₂NHPr, SO₂NHCF₃, COOH, COMe, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCOCH₂OCH₃, NHCO(CH₂)₂OCH₃, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH_{3,} NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMeNMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH_{2,} CONHCH(CH₃)CH₂OCH₃, CONHCH(C₂H₅)CH₂OCH₃, CONH(CH₂)₂OCH₃, COOH, CO₂CH₃, CO₂Et, CO₂Pr, CO₂isoPr, CO₂Bu, CO₂secBu, CO₂isoBu, CO₂tBu, CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂SO₂NHCH(CH₃)CH₂OCH_{3,} CH₂COtertBu, CH₂COCH₃, CH₂COOEt, C(CH₃)₂OCH₃, CHCH₃OCH₃, CHCF₃OCH₃, CHCF₃OC₂H₅, CHCHF₂OCH₃, C(CH₃)₂OH, CHCH₃OH, CHCHF₂OH, CHCF₃OH, CH₂C(CH₃)₂OCH₃, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, (CH₂)₄OMe, CH₂SMe, (CH₂)₂SMe, (CH₂)₃SMe, (CH₂)₄SMe, CH₂NAc₂, CH₂N(COCF₃)₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, CH₂COOPr, (CH₂)₂COOPr, CH₂COOisoPr, (CH₂)₂COOisoPr, CH₂COOtertBu, (CH₂)₂COOtertBu, CH₂COO(CH₂)₂OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl-(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazo-lidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclo-pentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (I):

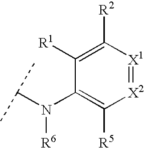

may be (2-oxo-2,3-dihydro-1H-indol-5-yl)aminoindol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino,

R⁶ represents hydrogen, Me, CH₂OCH₃, formyl, COMe, COOMe, COOEt, COOtertBu, COOBn, COCF₃, benzyl, 4-methoxybenzyl, C₂F₅OC(═O), CH₂CH═CH₂, CH₂C≡CH, SOCH₃, SO₂CH₃,

R⁷ represents hydrogen, cyano, methyl, CF₃, CFH₂,

R⁸ represents fluorine, chlorine, bromine, iodine, cyano, methyl, CF₃, CCl₃, CFH₂ or CF₂H

R⁹ represents hydrogen, Me, CH₂OCH₃, COMe, COOMe, COOEt, COOtertBu, COCF₃, benzyl, 4-methoxybenzyl,

R¹⁰ represents hydrogen, OEt, COOEt, 2-chlorophenyl, COOMe, COEt, COMe,

R¹¹ represents identical or different hydrogen, fluorine, chlorine,

and agrochemically active salts thereof as fungicides.

Special preference is given to using, as fungicides, compounds of the formula (I) in which one or more of the symbols have one of the meanings below:

X¹ represents nitrogen or CR³

X² represents nitrogen or CR⁴

A represents a direct bond, methylene or —CH(CH₃)—

R¹ to R⁵ independently of one another represent hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO₂Me, SO₂CF₃, SO₂Et, SO₂Pr, SO₂CH₂CH═CH_{2,} SO₂CH₂CN, SO₂CH₂C≡CH, SONHMe, SONMe₂, SO₂NHMe, SO₂NMe₂, SO₂NH_{2,} SO₂NHAc, SO₂NHPh, SO₂NHnBu, SO₂NHtBu, SO₂NEt₂, SO₂NHEt, SO₂NPr₂, COOH, COMe, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBu NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCOCH₂OCH₃, NHCO(CH₂)₂OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH_{3,} NCH₃COCH₃, acetyl(cyclopropyl)amino, [(1-methylcyclopropyl)carbonyl]amino, morpholin-1-yl, morpholin-4-yl-methyl, NHSO₂CH₃ NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCONHPr, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH_{2,} CONHCH(CH₃)CH₂OCH₃, CONH(CH₂)₂OCH₃, COOH, CO₂CH₃, CO₂Et, CO₂Pr, CO₂isoPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazol-idin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (I):

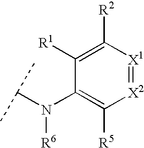

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzo-thiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino,

R⁶ represents hydrogen, Me, formyl, COMe, COOMe, COOEt, COOtertBu, COOBn, COCF₃, benzyl,

R⁷ represents hydrogen, methyl, CF₃,

R⁸ represents fluorine, chlorine, bromine, iodine, cyano, methyl, CF₃, CCl₃, CFH₂,

R⁹ represents hydrogen, Me,

R¹⁰ represents hydrogen, OEt, COOEt, 2-chlorophenyl,

R¹¹ represents identical or different hydrogen, fluorine, chlorine,

and agrochemically active salts thereof as fungicides.

Preference is furthermore given to using, as fungicides, compounds of the formula (I) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to using, as fungicides, compounds of the formula (I) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to using, as fungicides, compounds of the formula (I) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to using, as fungicides, compounds of the formula (I) in which one or more of the symbols have one of the meanings below:

X¹ represents nitrogen or CR³,

X² represents nitrogen or CR⁴,

A represents a direct bond, methylene or —CH(CH₃)—,

R¹ to R⁵ independently of one another represent COCl, CH═NOR^{12, CR}¹³═NOR¹², SF₅,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R¹ to R⁵ independently of one another represent CH═NOMe, C(CH₃)═NOMe, CH═NOEt, C(CH₃)═NOEt,

where the other substituents have one or more of the meanings mentioned above,

and also to the agrochemically active salts thereof.

Preference is furthermore given to using, as fungicides, compounds of the formula (I) in which one or more of the symbols have one of the meanings below:

A represents a direct bond, methylene or —CH(CH₃)—,

R¹⁰ represents CH₂OPh,

where the other substituents have one or more of the meanings mentioned above,

and also to the agrochemically active salts thereof.

Preference is furthermore given to using, as fungicides, compounds of the formula (I) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to using, as fungicides, compounds of the formula (I) in which one or more of the symbols have one of the meanings below:

X¹ represents CR³ and

X² represents CR⁴,

where the other substituents have one or more of the meanings mentioned above,

and also to the agrochemically active salts thereof.

Preference is furthermore given to using, as fungicides, compounds of the formula (I) in which one or more of the symbols have one of the meanings below:

X¹ represents nitrogen and

X² represents nitrogen,

where the other substituents have one or more of the meanings mentioned above,

and also to the agrochemically active salts thereof.

Preference is furthermore given to using, as fungicides, compounds of the formula (I) in which one or more of the symbols have one of the meanings below:

X¹ represents CR³ and

X² represents nitrogen,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R¹⁰ represents H or Me,

R^{11a,b,c} represents H,

X¹ represents CR³ and

X² represents CR⁴,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to using, as fungicides, compounds of the formula (I) in which one or more of the symbols have one of the meanings below:

R¹ represents H,

R⁵ represents H,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R¹ represents H,

R⁵ represents H,

X¹ represents CR³ and

X² represents CR⁴,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R⁸ represents chlorine, bromine, CF₃,

where the other substituents have one or more of the meanings mentioned above,

and also to the agrochemically active salts thereof.

Preference is furthermore given to using, as fungicides, compounds of the formula (I) in which one or more of the symbols have one of the meanings below:

R¹ to R⁵ independently of one another represent hydrogen, halogen, cyano, hydroxyl, nitro, an unsubstituted or substituted saturated, partially unsaturated or aromatic heterocyclic or carbocyclic five-, six- or seven-membered ring, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8,

where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,

where the other substituents have one or more of the meanings mentioned above,

and also to the agrochemically active salts thereof.

The radical definitions mentioned above can be combined with one another as desired. Moreover, individual definitions may not apply.

The invention also provides compounds of the formulae (Ia), (Ib), (Ic), (Id), (Ie) and (If).

Compounds of the formulae (Ia), (Ib), (Ic), (Id), (Ie) and (If) are highly suitable for controlling unwanted microorganisms. In particular, they have strong fungicidal activity and can be used both in crop protection and in the protection of materials.

a) Compounds of the formula (Ia)

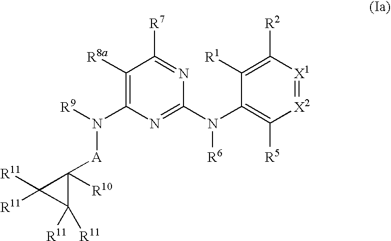

in which the symbols have the following meanings:

R^{8a} represents chlorine, iodine, CFH₂, CF₂H or CCl₃ and

X¹, X², A, R¹ to R⁷, R^{1-A}, R⁹, R¹⁰, R¹¹, R¹², R¹³ and R¹⁴ have the general, preferred, particularly preferred, very particularly preferred and especially preferred meanings given above, and also agrochemically active salts of these compounds, are preferred.

Preference is furthermore given to compounds of the formula (Ia) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (Ia) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (Ia) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to compounds of the formula (Ia) in which one or more of the symbols have one of the meanings below:

X¹ represents nitrogen or CR³,

X² represents nitrogen or CR⁴,

A represents a direct bond, methylene or —CH(CH₃)—,

R¹ to R⁵ independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (Ia) in which one or more of the symbols have one of the meanings below:

R¹ to R⁵ independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (Ia) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (Ia) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to agrochemically active salts thereof.

Preference is furthermore given to compounds of the formula (Ia) in which one or more of the symbols have one of the meanings below:

X¹ represents CR³ and

X² represents CR⁴,

where the other substituents have one or more of the meanings mentioned above,

and also to the agrochemically active salts thereof.

Preference is furthermore given to using compounds of the formula (Ia) in which one or more of the symbols have one of the meanings below:

X¹ represents nitrogen and

X² represents nitrogen,

where the other substituents have one or more of the meanings mentioned above,

and also to the agrochemically active salts thereof.

Preference is furthermore given to compounds of the formula (Ia) in which one or more of the symbols have one of the meanings below:

X¹ represents CR³ and

X² represents nitrogen,

where the other substituents have one or more of the meanings mentioned above,

and also to the agrochemically active salts thereof.

Preference is furthermore given to compounds of the formula (Ia) in which one or more of the symbols have one of the meanings below:

R¹⁰ represents H or Me,

R¹¹ represents H,

X¹ represents CR³ and

X² represents CR⁴,

where the other substituents have one or more of the meanings mentioned above,

and also to the agrochemically active salts thereof.

Preference is furthermore given to compounds of the formula (Ia) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (Ia) in which one or more of the symbols have one of the meanings below:

R¹ represents H,

R⁵ represents H,

where the other substituents have one or more of the meanings mentioned above,

and also to the agrochemically active salts thereof.

Preference is furthermore given to compounds of the formula (Ia) in which one or more of the symbols have one of the meanings below:

R¹ represents H,

R⁵ represents H,

X¹ represents CR³ and

X² represents CR⁴,

where the other substituents have one or more of the meanings mentioned above,

and also to the agrochemically active salts thereof.

Preference is furthermore given to compounds of the formula (Ia) in which one or more of the symbols have one of the meanings below:

R^{8a} represents chlorine,

where the other substituents have one or more of the meanings mentioned above,

and also to the agrochemically active salts thereof.

The radical definitions mentioned above can be combined with one another as desired. Moreover, individual definitions may not apply.

b) Compounds of the formula (Ib)

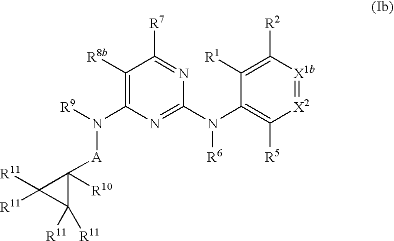

in which the symbols have the following meanings:

X^{1b} represents nitrogen or C—R^{3b}, and

R^{3b} represents hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent to one another, OR¹², SR¹², SOR¹², SO₂CF₃, SO₂BuBu, SO₂secBu, SO₂isoBu, SO₂-tertBu, SO₂-pentyl, SO₂neopentyl, SO₂CH₂CH═CH₂, SO₂CH₂CN, SO₂CH₂C≡CH, SON(R¹²)₂, SO₂NHMe, SO₂NMe₂, SO₂NH_{2,} SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl; where m=1-8,

where additionally or independently thereof in each case two adjacent radicals R², R^{3b} or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another; and

R^{8b} represents cyano and

X², A, R¹, R², R⁴ to R⁷, R^{1-A}, R⁹, R¹⁰, R¹¹, R¹², R¹³ and R¹⁴ have thegeneral meanings given above, and also agrochemically active salts of these compounds are preferred.

The formula (Ib) provides a general definition of the compounds according to the invention.

Preference is given to compounds of the formula (Ib) in which one or more of the symbols have one of the preferred meanings listed below, i.e.

X^{1b} represents nitrogen or C—R^{3b}, and

R^{3b} represents hydrogen, fluorine, chlorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF_{3,} OCF₂CF₂H_{,} OCH₂CF₂H_{,} OCH₂CF_{3,} OCH₂CH₂N(C₂H₅)_{2,} OCH₂CH₂N(CH₃)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SCFH₂, SOMe, SO-Et, SO—Pr, SOisoPr, SOBuBu, SOsecBu, SOisoBu, SO-tBu, SO-pentyl, SOneopentyl, SO₂CF₃, SO₂BuBu, SO₂secBu, SO₂isoBu, SO₂-tertBu, SO₂-pentyl, SO₂neopentyl, SO₂CH₂CH═CH₂, SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtertBu, SONHpentyl, SONBu₂, SONHCF_{3,} SON(CF₃)₂, SO₂NHMe, SO₂NMe₂, SO₂NH_{2,} SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} COMe, COEt, COPr, COisoPr, COBu, COsecBu, COisoBu, COtertBu, COcyclopropyl, COCHF₂, COCH₂F, COCF₃, NHCOOMe, NHCOOisoPr, NHCOO-tertBu, NHCOOnBuBu, NHCOOpentyl, NHCOOcycloproyl, NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(CH₃)COCH₃, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, N-sBu₂, N-tBu₂, NHMe, NH₂, NHtertBu, NHsBu, NHEt, NHPr, NHisoPr, NHBu, NHisoBu, NHsecBu, cyclopropylamino, NH(Et)cyclopropyl, NH(Et)cyclopropyl, NHCH(CH₃)CH₂OCH_{3,} NHCH(CH₃)CH₂OEt_{,} NCH₃COCH₃, NEtCOCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tBu, NMeSOtertBu, NMeSO₂tBu, NHSOsBu, NHSO₂sBu, NMeSOsBu, NMeSO₂sBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me) isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)_{2,} OSO₂NEt_{2,} CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, CO₂(CH₂)₂OCH₃, CO₂(CH₂)₂OEt, COCH₂N(CH₃)₂, CO₂(CH₂)₃OCH₃, COCH₂NEt₂, CO₂(CH₂)₃OEt, CH₂SO₂NHMe, CH₂SO₂NHisoPr, CH₂SO₂NHPr, CH₂SO₂NHtertBu, CH₂SO₂NHEt, CH₂COtertBu, CH₂COCH₃, CH₂COEt, CH₂COOEt, CH₂COOMe, C(CH₃)₂OCH₃, CHCH₃OCH₃, CHCF₃OCH₃, CHCF₃OC₂H₅, C(CH₃)₂OEt, CHCH₃OEt, CHCF₃OEt, C(CH₃)₂OH, CHCH₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₂SH, (CH₂)₃SH, (CH₂)₄SH, CH₂SMe, (CH₂)₂SMe, (CH₂)₃SMe, (CH₂)₄SMe,═CH₂SEt, (CH₂)₂SEt, (CH₂)₃SEt, (CH₂)₄SEt, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH2)3OCH3, O-cyclopentyl, CH2NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl and

R^{8b} represents cyano and

X², A, R¹, R², R⁴ to R⁷, R^{1-A}, R⁹, R¹⁰, R¹¹, R¹², R¹³ and R¹⁴ have the preferred meanings given above, and also to agrochemically active salts of these compounds.

Particular preference is given to compounds of the formula (Ib) in which one or more of the symbols have one of the particularly preferred meanings listed below, i.e.

X^{1b} represents nitrogen or C—R^{3b}, and

R^{3b} represents hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OH, O—(CH₂)₂OCH₃, O—(CH₂)₃OH, O—(CH₂)₃OCH₃, O-cyclopentyl, OCF₃, OCF₂H, OCF₂CF_{3,} OCF₂CF₂H, OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu SO₂CF₃, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SONHEt, SONEt₂, SONHPr, SONPr₂, SONHnBu, SONBu₂, SONHCF_{3,} SON(CF₃)₂, SO₂NHMe, SO₂NMe₂, SO₂NH_{2,} SO₂NHAc, SO₂NHPh, SO₂NHnBu, SO₂NEt₂, SO₂NHEt, SO₂NPr₂, SO₂NHPr, SO₂NHCF₃, SO₂N(CF₃)₂, SO₂NH(CH₂)₃NMe_{2,} SO₂NHCH₂CH═CH_{2,} COMe, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH_{3,} NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHsecBu, CONHisoBu, CONHtBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONH(CH₂)₂OH, CONH(CH₂)₃OCH₃, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH,═CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, C(CH₃)₂OH, CHCH₃OH, CHCF₃OH, CH₂C(CH₃)₂OCH₃, CHCHF₂OCH₃, CHCHF₂OH, CH₂OH, CH₂NHCOOBn, CH₂NHCOOtertBu, CH₂SO₂CH₃, CH₂NH(CH₂)₂OCH₃, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe,═CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, 0-(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tent-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino , 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R^{3b} or R⁴, if appropriate via R¹² or R¹³, form a cycle, the following subunit of the general formula (Ib):

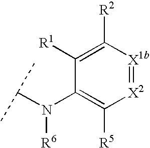

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8b} represents cyano and

X², A, R¹, R², R⁴ to R⁷, R^{1-A}, R⁹, R¹⁰, R¹¹, R¹², R¹³ and R¹⁴ have the particularly preferred meanings given above, and also to agrochemically active salts of these compounds.

Very particular preference is given to compounds of the formula (Ib) in which one or more of the symbols have one of the very particularly preferred meanings listed below, i.e.

X^{1b} represents nitrogen or C—R^{3b}, and

R^{3b} represents hydrogen, fluorine, chlorine, bromine, iodine, cyano, hydroxyl, nitro, OMe, OEt, OPr, OisoPr, OBu, OsecBu, OisoBu, OtertBu, (CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu, SO₂CF₃, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SO₂NHMe, SO₂NMe₂, SO₂NH_{2,} SO₂NHAc, SO₂NHPh, SO₂NHnBu, SO₂NEt₂, SO₂NHEt, SO₂NPr₂, SO₂NHPr, SO₂NHCF₃, COOH, COMe, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCOCH₂OCH₃, NHCO(CH₂)₂OCH₃, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMeNMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH_{2,} CONHCH(CH₃)CH₂OCH₃, CONHCH(C₂H₅)CH₂OCH₃, CONH(CH₂)₂OCH₃, COOH, CO₂CH₃, CO₂Et, CO₂Pr, CO₂isoPr, CO₂Bu, CO₂secBu, CO₂isoBu, CO₂tBu, CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, CHCHF₂OCH₃, C(CH₃)₂OH, CHCH₃OH, CHCHF₂OH, CHCF₃OH, CH₂C(CH₃)₂OCH₃, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, (CH₂)₄OMe, CH₂SMe, (CH₂)₂SMe, (CH₂)₃SMe, (CH₂)₄SMe, CH₂NAc₂, CH₂N(COCF₃)₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, CH₂COOPr, (CH₂)₂COOPr, CH₂COOisoPr, (CH₂)₂COOisoPr, CH₂COOtertBu, (CH₂)₂COOtertBu, CH₂COO(CH₂)₂OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O-(CH2)3OCH3, O-cyclopentyl, CH2NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R^{3b} or R⁴, if appropriate via R¹² or R¹³, form a cycle, the following subunit of the general formula (Ib):

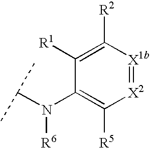

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8b} represents cyano and

X², A, R¹, R², R⁴ to R⁷, R^{1-A}, R⁹, R¹⁰, R¹¹, R¹², R¹³ and R¹⁴ have the very particularly preferred meanings given above, and also to agrochemically active salts of these compounds.

Special preference is given to compounds of the formula (Ib) in which one or more of the symbols have one of the especially preferred meanings listed below, i.e.

X^{1b} represents nitrogen or C—R^{3b}, and

R^{3b} represents hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO₂CF₃,—SO₂CH₂CH═CH_{2,} SO₂CH₂CN, SO₂CH₂C≡CH, SONHMe, SONMe₂, SO₂NHMe, SO₂NMe₂, SO₂NH_{2,} SO₂NHAc, SO₂NHPh, SO₂NHnBu, SO₂NEt₂, SO₂NHEt, SO₂NPr₂, COOH, COMe, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCOCH₂OCH₃, NHCO(CH₂)₂OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, morpholin-1-yl, morpholin-4-yl-methyl, NHSO₂CH₃ NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH_{2,} CONHCH(CH₃)CH₂OCH₃, CONH(CH₂)₂OCH₃, COOH, CO₂CH₃, CO₂Et, CO₂Pr, CO₂isoPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R^{3b} or R⁴, if appropriate via R¹² or R¹³, form a cycle, the following subunit of the general formula (Ib):

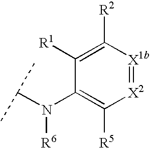

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8b} represents cyano and

X², A, R¹, R², R⁴ to R⁷, R^{1-A}, R⁹, R¹⁰, R¹¹, R¹², R¹³ and R¹⁴ have the especially preferred meanings given above, and also to agrochemically active salts of these compounds.

Preference is furthermore given to compounds of the formula (Ib) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (Ib) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (Ib) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to compounds of the formula (Ib) in which one or more of the symbols have one of the meanings below:

X^{1b} represents nitrogen or CR^{3b},

X² represents nitrogen or CR⁴,

A represents a direct bond, methylene or —CH(CH₃)—,

R¹ to R⁵ and R^{3b} independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (Ib) in which one or more of the symbols have one of the meanings below:

R¹ to R⁵ and R^{3b} independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (Ib) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (Ib) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to agrochemically active salts thereof.

Preference is furthermore given to compounds of the formula (Ib) in which one or more of the symbols have one of the meanings below:

X^{1b} represents CR^{3b} and

X² represents CR⁴,

where the other substituents have one or more of the meanings mentioned above,

and also to the agrochemically active salts thereof.

Preference is furthermore given to compounds of the formula (Ib) in which one or more of the symbols have one of the meanings below:

X^{1b} represents nitrogen and

X² represents nitrogen,

where the other substituents have one or more of the meanings mentioned above,

and also to the agrochemically active salts thereof.

Preference is furthermore given to compounds of the formula (Ib) in which one or more of the symbols have one of the meanings below:

X^{1b} represents CR^{3b} and

X² represents nitrogen,

where the other substituents have one or more of the meanings mentioned above,

and also to the agrochemically active salts thereof.

Preference is furthermore given to compounds of the formula (Ib) in which one or more of the symbols have one of the meanings below:

R¹⁰ represents H or Me,

R¹¹ represents H,

X^{1b} represents CR^{3b} and

X² represents CR⁴,

where the other substituents have one or more of the meanings mentioned above,

and also to the agrochemically active salts thereof.

Preference is furthermore given to compounds of the formula (Ib) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (Ib) in which one or more of the symbols have one of the meanings below:

R¹ represents H,

R⁵ represents H,

where the other substituents have one or more of the meanings mentioned above,

and also to the agrochemically active salts thereof.

Preference is furthermore given to compounds of the formula (Ib) in which one or more of the symbols have one of the meanings below:

R¹ represents H,

R⁵ represents H,

X^{1b} represents CR^{3b} and

X² represents CR⁴,

where the other substituents have one or more of the meanings mentioned above,

and also to the agrochemically active salts thereof.

The radical definitions mentioned above can be combined with one another as desired. Moreover, individual definitions may not apply.

c) Compounds of the formula (Ic)

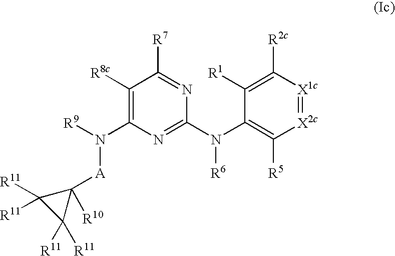

in which the symbols have the following meanings:

X^{1c} represents nitrogen or C—R^{3c},

X^{2c} represents nitrogen or C—R^{4c},

R^{2c} and R^{4c} independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent to one another, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF_{3,} OCF₂CF₂H_{,} OCH₂CF₂H_{,} OCH₂CF_{3,} OCH₂CH₂N(C₂H₅)_{2,} OCH₂CH₂N(CH₃)_{2,} OCH(CH₃)CH₂OCH₃, SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl; where m=1-8,

where additionally or independently thereof in each case two adjacent radicals R^{2c}, R^{3c} or R^{4c}, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another;

R^{3c} represents hydrogen, fluorine, chlorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF_{3,} OCF₂CF₂H_{,} OCH₂CF₂H_{,} OCH₂CF_{3,} OCH₂CH₂N(C₂H₅)_{2,} OCH₂CH₂N(CH₃)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SCFH₂, SOMe, SO-Et, SO—Pr, SOisoPr, SOBuBu, SOsecBu, SOisoBu, SO-tBu, SO-pentyl, SOneopentyl, SO₂Me, SO₂CF₃, SO₂Et, SO₂Pr, SO₂isoPr, SO₂BuBu, SO₂secBu, SO₂isoBu, SO₂-tBu, SO₂-pentyl, SO₂neopentyl, SO₂CH₂CH═CH_{2,} SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtertBu, SONHpentyl, SONBu₂, SONHCF_{3,} SON(CF₃)₂, SO₂NHMe, SO₂NMe₂, SO₂NH_{2,} SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} COMe, COEt, COPr, COisoPr, COBu, COsecBu, COisoBu, COtertBu, COcyclopropyl, COCHF₂, COCH₂F, COCF₃, NHCOOMe, NHCOOisoPr, NHCOO-tertBu, NHCOOnBuBu, NHCOOpentyl, NHCOOcycloproyl, NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, N-sBu₂, N-tBu₂, NHMe, NH₂, NHtertBu, NHsBu, NHEt, NHPr, NHisoPr, NHBu, NHisoBu, NHsecBu, cyclopropylamino, NH(Et)cyclopropyl, NH(Et)cyclopropyl, NHCH(CH₃)CH₂OCH_{3,} NHCH(CH₃)CH₂OEt_{,} NCH₃COCH₃, NEtCOCH₃, acetyl(cyclopropyl)amino, [(1-methylcyclopropyl)carbonyl]amino, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tBu, NMeSOtertBu, NMeSO₂tBu, NHSOsBu, NHSO₂sBu, NMeSOsBu, NMeSO₂sBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me)isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)_{2,} OSO₂NEt_{2,} CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, CO₂(CH₂)₂OCH₃, CO₂(CH₂)₂OEt, COCH₂N(CH₃)₂, CO₂(CH₂)₃OCH₃, COCH₂NEt₂, CO₂(CH₂)₃OEt, CH₂SO₂NHMe, CH₂SO₂NHisoPr, CH₂SO₂NHPr, CH₂SO₂NHtertBu, CH₂SO₂NHEt, CH₂COtertBu, CH₂COCH₃, CH₂COEt, CH₂COOEt, CH₂COOMe, C(CH₃)₂OCH₃, CHCH₃OCH₃, CHCF₃OCH₃, CHCF₃OC₂H₅, C(CH₃)₂OEt, CHCH₃OEt, CHCF₃OEt, C(CH₃)₂OH, CHCH₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₂SH, (CH₂)₃SH, (CH₂)₄SH, CH₂SMe, (CH₂)₂SMe, (CH₂)₃SMe, (CH₂)₄SMe,═CH₂SEt, (CH₂)₂SEt, (CH₂)₃SEt, (CH₂)₄SEt, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

R^{8c} represents fluorine and

A, R¹, R⁵ to R⁷, R^{1-A}, R⁹, R¹⁰, R¹¹, R¹², R¹³ and R¹⁴ have the general meanings given above, and also agrochemically active salts of these compounds are preferred.

The formula (Ic) provides a general definition of the compounds according to the invention.

Preference is given to compounds of the formula (Ic) in which one or more of the symbols have one of the preferred meanings listed below, i.e.

X^{1c} represents nitrogen or C—R^{3c},

X^{2c} represents nitrogen or C—R^{4c},

R^{2c}, R^{4c} and R^{3c} independently of one another represent hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OH, O—(CH₂)₂OCH₃, O—(CH₂)₃OH, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu,═SO₂Me, SO₂CF₃, SO₂Et, SO₂Pr, SO₂isoPr, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SONHEt, SONEt₂, SONHPr, SONPr₂, SONHnBu, SONBu₂, SONHCF_{3,} SON(CF₃)₂,═SO₂NHMe, SO₂NMe₂, SO₂NH_{2,} SO₂NHAc, SO₂NHPh, SO₂NHnBu, SO₂NEt₂, SO₂NHEt, SO₂NPr₂, SO₂NHPr, SO₂NHCF₃, SO₂N(CF₃)₂, SO₂NH(CH₂)₃NMe_{2,} SO₂NHCH₂CH═CH_{2,} COMe, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, NHSO₂CH₃ NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHsecBu, CONHisoBu, CONHtertBu, CONHCH₂CH═CH_{2,} CONHCH(CH₃)CH₂OH, CONHCH(CH₃)CH₂OCH₃, CONHCH(C₂H₅)CH₂OCH₃, CONH(CH₂)₂OCH₃, CONH(CH₂)₂OH, CONH(CH₂)₃OCH₃, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH,═CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, C(CH₃)₂OH, CHCH₃OH, CHCF₃OH, CH₂C(CH₃)₂OCH₃, CHCHF₂OCH₃, CHCHF₂OH, CH₂OH, CH₂NHCOOBn, CH₂NHCOOtertBu, CH₂SO₂CH₃, CH₂NH(CH₂)₂OCH₃, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe,═CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH_{3,} CH₂COO(CH₂)₃OH, CH₂COO(CH₂)₃OCH_{3,} CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R^{2c}, R^{3c} or R^{4c}, if appropriate via R¹² or R¹³, form a cycle, the following subunit of the general formula (Ic):

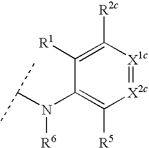

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8c} represents fluorine and

A, R¹, R⁵ to R⁷, R^{1-A}, R⁹, R¹⁰, R¹¹, R¹², R¹³ and R¹⁴ have the preferred meanings given above, and also agrochemically active salts of these compounds are preferred.

Particular preference is given to compounds of the formula (Ic) in which one or more of the symbols have one of the particularly preferred meanings listed below, i.e.

C^{1c} represents nitrogen or C—R^{3c},

X^{2c} represents nitrogen or C—R^{4c},

R^{2c}, R^{4c} and R^{3c} independently of one another represent hydrogen, fluorine, chlorine, bromine, iodine, cyano, hydroxyl, nitro, OMe, OEt, OPr, OisoPr, OBu, OsecBu, OisoBu, OtertBu, (CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu, SO₂Me, SO₂CF₃, SO₂Et, SO₂Pr, SO₂isoPr, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SO₂NHMe, SO₂NMe₂, SO₂NH_{2,} SO₂NHAc, SO₂NHPh, SO₂NHnBu, SO₂NEt₂, SO₂NHEt, SO₂NPr₂, SO₂NHPr, SO₂NHCF₃, COOH, COMe, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCOCH₂OCH₃, NHCO(CH₂)₂OCH₃, NHCO(CH₂)₃OCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH_{3,} NCH₃COCH₃, acetyl(cyclopropyl)amino, [(1-methylcyclopropyl)carbonyl]amino, NHSO₂CH₃ NHSOMe, NHSO₂Me, NHSOEt, NHSO₂Et, NMeSOMeNMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH_{2,} CONHCH(CH₃)CH₂OCH₃, CONHCH(C₂H₅)CH₂OCH₃, CONH(CH₂)₂OCH₃, COOH, CO₂CH₃, CO₂Et, CO₂Pr, CO₂isoPr, CO₂Bu, CO₂secBu, CO₂isoBu, CO₂tBu, CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, CHCHF₂OCH₃, C(CH₃)₂OH, CHCH₃OH, CHCHF₂OH, CHCF₃OH, CH₂C(CH₃)₂OCH₃, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, (CH₂)₄OMe, CH₂SMe, (CH₂)₂SMe, (CH₂)₃SMe, (CH₂)₄SMe, CH₂NAc₂, CH₂N(COCF₃)₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, CH₂COOPr, (CH₂)₂COOPr, CH₂COOisoPr, (CH₂)₂COOisoPr, CH₂COOtertBu, (CH₂)₂COOtertBu, CH₂COO(CH₂)₂OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH2NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl-(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H -tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R^{2c}, R^{3c} or R^{4c}, if appropriate via R¹² or R¹³, form a cycle, the following subunit of the general formula (Ic):

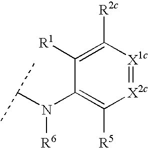

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8c} represents fluorine and

A, R¹, R⁵ to R⁷, R^{1-A}, R⁹, R¹⁰, R¹¹, R¹², R¹³ and R¹⁴ have the particularly preferred meanings given above, and also agrochemically active salts of these compounds are preferred.

Very particular preference is given to compounds of the formula (Ic) in which one or more of the symbols have one of the very particularly preferred meanings listed below, i.e.)

X^{1c} represents nitrogen or C—R^{3c},

X^{2c} represents nitrogen or C—R^{4c},

R^{2c}, R^{3c} and R^{4c} independently of one another represent hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF_{3,} OCF₂CF₂H_{,} OCH₂CH₂N(C₂H₅)_{2,} OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO₂Me, SO₂CF₃, SO₂Et, SO₂Pr, SO₂CH₂CH═CH_{2,} SO₂CH₂CN, SO₂CH₂C≡CH, SONHMe, SONMe₂, SO₂NHMe, SO₂NMe_{2,} SO₂NH_{2,} SO₂NHAc, SO₂NHPh, SO₂NHnBu, SO₂NEt₂, SO₂NHEt, SO₂NPr₂, COOH, COMe, SO₂NH(CH₂)₃NMe_{2,} SO₂NHCH₂CH═CH₂, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, NHSO₂CH₃ NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONH(CH₂)₂OCH₃, COOH, CO₂CH₃, CO₂Et, CO₂Pr, CO₂isoPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R^{2c}, R^{3c} or R^{4c}, if appropriate via R¹² or R¹³, form a cycle, the following subunit of the general formula (Ic):

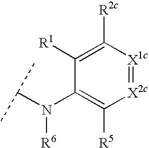

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8c} represents fluorine and

A, R¹, R⁵ to R⁷, R^{1-A}, R⁹, R¹⁰, R¹¹, R¹², R¹³ and R¹⁴ have the very particularly preferred meanings given above, and also agrochemically active salts of these compounds are preferred.

Preference is furthermore given to compounds of the formula (Ic) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (Ic) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (Ic) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to compounds of the formula (Ic) in which one or more of the symbols have one of the meanings below:)

X^{1c} represents nitrogen or CR^{3c},

X^{2c} represents nitrogen or CR^{4c},

A represents a direct bond, methylene or —CH(CH₃)—,

R¹ to R⁵ and R^{3c} to R^{4c} independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (Ic) in which one or more of the symbols have one of the meanings below:

R¹ to R⁵ and R^{3c} to R^{4c} independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (Ic) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (Ic) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to agrochemically active salts thereof.

Preference is furthermore given to compounds of the formula (Ic) in which one or more of the symbols have one of the meanings below:

X^{1c} represents CR^{3c} and

X^{2c} represents CR^{4c},

where the other substituents have one or more of the meanings mentioned above,

and also to the agrochemically active salts thereof.

Preference is furthermore given to compounds of the formula (Ic) in which one or more of the symbols have one of the meanings below:

X^{1c} represents nitrogen and

X^{2c} represents nitrogen,

where the other substituents have one or more of the meanings mentioned above,

and also to the agrochemically active salts thereof.

Preference is furthermore given to compounds of the formula (Ic) in which one or more of the symbols have one of the meanings below:

X^{1c} represents CR^{3c} and

X^{2c} represents nitrogen,

where the other substituents have one or more of the meanings mentioned above,

and also to the agrochemically active salts thereof.

Preference is furthermore given to compounds of the formula (Ic) in which one or more of the symbols have one of the meanings below:

R¹⁰ represents H or Me,

R¹¹ represents H,

X^{1c} represents CR^{3c} and

X^{2c} represents CR^{4c},

where the other substituents have one or more of the meanings mentioned above,

and also to the agrochemically active salts thereof.

Preference is furthermore given to compounds of the formula (Ic) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (Ic) in which one or more of the symbols have one of the meanings below:

R¹ represents H,

R⁵ represents H,

where the other substituents have one or more of the meanings mentioned above,

and also to the agrochemically active salts thereof.

Preference is furthermore given to compounds of the formula (Ic) in which one or more of the symbols have one of the meanings below:

R¹ represents H,

R⁵ represents H,

X^{1c} represents CR^{3c} and

X^{2c} represents CR^{4c},

where the other substituents have one or more of the meanings mentioned above,

and also to the agrochemically active salts thereof.

The radical definitions mentioned above can be combined with one another as desired. Moreover, individual definitions may not apply.

d) Compounds of the formula (Id)

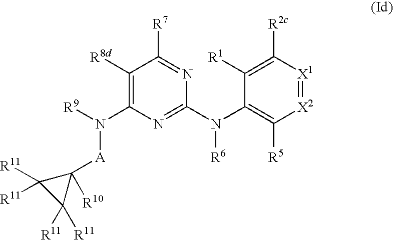

in which the symbols have the following meanings:

R¹ to R⁵ correspond to the above definitions, except for the following cases: either X¹ represents CR³ and R² and R³, in the following subunit of the general formula (Id),

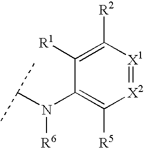

form (2-oxo-2,3-dihydro-1H-indol-5-yl)amino,

or X¹ represents CR³ and X² represents CR⁴ and R⁴ and R³, in the above subunit of the general formula (Id), also form (2-oxo-2,3-dihydro-1H-indol-5-yl)amino; and

R^{8d} represents CF₃ and

X¹, X², A, R⁶, R⁷ R^{1-A}, R⁹, R¹⁰, R¹¹, R¹², R¹³ and R¹⁴ have the general, preferred, particularly preferred, very particularly preferred and especially preferred meanings given above, and also agrochemically active salts of these compounds, are preferred.

Preference is furthermore given to compounds of the formula (Id) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (Id) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (Id) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to compounds of the formula (Id) in which one or more of the symbols have one of the meanings below:

X¹ represents nitrogen or CR³,

X² represents nitrogen or CR⁴,

A represents a direct bond, methylene or —CH(CH₃)—,

R¹ to R⁵ independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (Id) in which one or more of the symbols have one of the meanings below:

R¹ to R⁵ independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (Id) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (Id) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to agrochemically active salts thereof.

Preference is furthermore given to compounds of the formula (Id) in which one or more of the symbols have one of the meanings below:

X¹ represents CR³ and

X² represents CR⁴,

where the other substituents have one or more of the meanings mentioned above,

and also to the agrochemically active salts thereof.

Preference is furthermore given to compounds of the formula (Id) in which one or more of the symbols have one of the meanings below:

X¹ represents nitrogen and

X² represents nitrogen,

where the other substituents have one or more of the meanings mentioned above,

and also to the agrochemically active salts thereof.

Preference is furthermore given to compounds of the formula (Id) in which one or more of the symbols have one of the meanings below:

X¹ represents CR³ and

X² represents nitrogen,

where the other substituents have one or more of the meanings mentioned above,

and also to the agrochemically active salts thereof.

Preference is furthermore given to compounds of the formula (Id) in which one or more of the symbols have one of the meanings below:

R¹⁰ represents H or Me,

R¹¹ represents H,

X¹ represents CR³ and

X² represents CR⁴,

where the other substituents have one or more of the meanings mentioned above,

and also to the agrochemically active salts thereof.

Preference is furthermore given to compounds of the formula (Id) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (Id) in which one or more of the symbols have one of the meanings below:

R¹ represents H,

R⁵ represents H,

where the other substituents have one or more of the meanings mentioned above,

and also to the agrochemically active salts thereof.

Preference is furthermore given to compounds of the formula (Id) in which one or more of the symbols have one of the meanings below:

R¹ represents H,

R⁵ represents H,

X¹ represents CR³ and

X² represents CR⁴,

where the other substituents have one or more of the meanings mentioned above,

and also to the agrochemically active salts thereof.

The radical definitions mentioned above can be combined with one another as desired. Moreover, individual definitions may not apply.

e) Compounds of the formula (Ie)

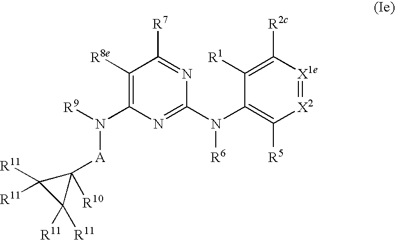

in which the symbols have the following meanings:

X^{1e} represents nitrogen or C—R^{3e},

R^{3e} represents hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent to one another, OR¹², SR¹², SOR¹², SO₂R¹², SON(R¹²)₂, SO₂NHMe, SO₂NMe₂, SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} OSO₂N(R¹²)₂, COEt, COPr, COisoPr, COBu, COsecBu, COisoBu, COtertBu, COcyclopropyl, COCHF₂, COCH₂F, COCF₃, NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8,

where additionally or independently thereof in each case two adjacent radicals R², R^{3e} or R⁴, if appropriate via R¹² or R¹³, together may form a cycle which, in the following subunit of the general formula (Ie):

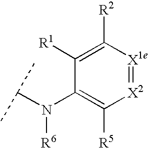

is (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3 ,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino;

R^{8e} represents Br and

X², A, R¹, R², R⁴ to R⁷, R^{1-A}, R⁹, R¹⁰, R¹¹, R¹², R¹³ and R¹⁴ have the general meanings given above, and also agrochemically active salts of these compounds are preferred.

Preference is given to compounds of the formula (Ie) in which one or more of the symbols have one of the preferred meanings listed below, i.e.

X^{1e} represents nitrogen or C—R^{3e},

R^{3e} represents hydrogen, fluorine, chlorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF₃, OCF₂CF₂H, OCH₂CF₂H, OCH₂CF₃, OCH₂CH₂N(C₂H₅)₂, OCH₂CH₂N(CH₃)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SCFH₂, SOMe, SO-Et, SO—Pr, SOisoPr, SOBuBu, SOsecBu, SOisoBu, SO-tBu, SO-pentyl, SOneopentyl, SO₂Me, SO₂CF₃, SO₂Et, SO₂Pr, SO₂isoPr, SO₂BuBu, SO₂secBu, SO₂isoBu, SO₂-tBu, SO₂-pentyl, SO₂neopentyl, SO₂CH₂CH═CH₂, SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtBu, SONHpentyl, SONBu₂, SONHCF₃, SON(CF₃)₂, SO₂NHMe, SO₂NMe₂, SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe₂, SO₂NH(CH₂)₃NEt₂, SO₂NHCH₂CH═CH₂, COEt, COPr, COisoPr, COBu COsecBu, COisoBu, COtertBu, COcyclopropyl, COCHF₂, COCH₂F, COCF₃, NHCOOMe, NHCOOisoPr, NHCOO-tertBu, NHCOOnBuBu, NHCOOpentyl, NHCOOcycloproyl, NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(CH₃)COCH₃, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, N-sBu₂, N-tBu₂, NHMe, NH₂, NHtertBu, NHsBu, NHEt, NHPr, NHisoPr, NHBu, NHisoBu, NHsecBu, cyclopropylamino, NH(Et)cyclopropyl, NH(Et)cyclopropyl, NHCH(CH₃)CH₂OCH₃, NHCH(CH₃)CH₂OEt, NCH₃COCH₃, NEtCOCH₃, acetyl(cyclopropyl)amino, [(1-methylcy clopropyl)carbonyl]amino, piperazin-1-yl, 4-methylpiperazin-1-yl, morpholin-1-yl, morpholin-4-yl-methyl, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tBu, NMeSOtertBu, NMeSO₂tBu, NHSOsBu, NHSO₂sBu, NMeSOsBu, NMeSO₂sBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me)isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)₂, OSO₂NEt₂, CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH₂, CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, CO₂(CH₂)₂OCH₃, CO₂(CH₂)₂OEt, COCH₂N(CH₃)₂, CO₂(CH₂)3OCH₃, COCH₂NEt₂, CO₂(CH₂)₃OEt, CH₂SO₂NHMe, CH₂SO₂NHisoPr, CH₂SO₂NHPr, CH₂SO₂NHtertBu, CH₂SO₂NHEt, CH₂SO₂NHisoPr, CH₂SO₂NHPr, CH₂SO₂NHtertBu, CH₂COtertBu, CH₂COCH₃, CH₂COEt, CH₂COOEt, CH₂COOMe, C(CH₃)₂OCH₃, CHCH₃OCH₃, CHCF₃OCH₃, C(CH₃)₂OEt, CHCH₃OEt, CHCF₃OEt, C(CH₃)₂OH, CHCH3OH, CHCF₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₃SH, (CH₂)₃SH, (CH₂)₄SH, CH₂SMe, (CH₂)₂SMe, (CH₂)₃SMe, (CH₂)₄SMe,═CH₂SEt, (CH₂)₂SEt, (CH₂)₃SEt, (CH₂)₄SEt, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH₃, CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tent-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxo cyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R^{3e} or R⁴, if appropriate via R¹² or R¹³, form a cycle, the following subunit of the general formula (Ie):

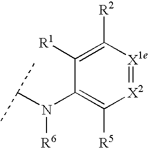

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)aminoamino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino; and

R^{8e} represents Br and

X², A, R¹, R², R⁴ to R⁷, R^{1-A}, R⁹, R¹⁰, R¹¹, R¹², R¹³ and R¹³ and R¹⁴ have the preferred meanings given above, and also to agrochemically active salts of these compounds.

Particular preference is given to compounds of the formula (Ie) in which one or more of the symbols have one of the particularly preferred meanings listed below, i.e.

X^{1e} represents nitrogen or C—R^{3e},

R^{3e} represents hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OH, O—(CH₂)₂OCH₃, O—(CH₂)₃OH, O—(CH₂)₃OCH₃, O-cyclopentyl, OCF₃, OCF₂H, OCF₂CF₃, OCF₂CF₂H, OCH₂CH₂N(C₂H₅)_{2,} OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu, ═SO₂Me, SO₂CF₃, SO₂Et, SO₂Pr, SO₂isoPr, SO₂BuBu, SO₂secBu, SO₂isoBu, SO₂-tBu, SO₂CH₂CH═CH₂, SO₂CH₂CN, SO₂CH₂CCH, SONHMe, SONMe₂, SONHEt, SONEt₂, SONHPr, SONPr₂, SONHnBu, SONBu₂, SONHCF₃, SON(CF₃)₂, ═SO₂NHMe, SO₂NMe₂, SO₂NHAc, SO₂NHPh, SO₂NHnBu, SO₂NEt₂, SO₂NHEt, SO₂NPr₂, SO₂NHPr, SO₂NHCF₃, SO₂N(CF₃)₂, SO₂NH(CH₂)₃NMe₂, SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₂, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methyl-cyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCOMe, OCOEt, OCOPr, OCOisoPr, OCOBu, OCOsecBu, OCOisoBu, OCOtertBu, OSO₂N(CH₃)₂, CONHEt, CONEt₂, CONHCH₃, CON(CH₃)₂, CONHPr, CONHisoPr, CONHPh, COCH₂CN, CONPr₂, CONHBu, CONHsecBu, CONHisoBu, CONHtertBu, CONHCH₂CH═CH₂, CONHCH(CH₃)CH₂OH, CONHCH(CH₃)CH₂OCH₃, CONHCH(C₂H₅)CH₂OCH₃, CONH(CH₂)₂OCH₃, CONH(CH₂)₂OH, CONH(CH₂)₃OCH₃, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, ═CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, C(CH₃)₂OH, CHCH₃OH, CHCF₃OH, CH₂C(CH₃)₂OCH₃, CHCHF₂OCH₃, CHCHF₂OH, CH₂OH, CH₂NHCOOBn, CH₂NHCOOtertBu, CH₂SO₂CH₃, CH₂NH(CH₂)₂OCH₃, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe,═CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH₃, CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylp entyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chloro cyclopropyl, cyclobutyl, cyclopentyl, cyclohexyl, CF₃, CF₂H, CCl₃, C₂F₅, C₃F₇, CF(CF₃)₂, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)-amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxo-imidazolidin-1-yl, 2,3-dimethyl-5-oxo-2,5-dihydro-1H-pyrazol-1-yl, thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R^{3e} or R⁴, if appropriate via R¹² or R¹³, form a cycle, the following subunit of the general formula (Ie):

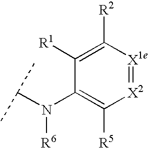

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino; and

R^{8e} represents Br and

X², A, R¹, R², R⁴ to R⁷, R^{1-A}, R⁹, R¹⁰, R¹¹, R¹², R¹³ and R¹⁴ have the particularly preferred meanings given above, and also to agrochemically active salts of these compounds.

Very particular preference is given to compounds of the formula (Ie) in which one or more of the symbols have one of the very particularly preferred meanings listed below, i.e.

X^{1e} represents nitrogen or C—R^{3e},

R^{3e} represents hydrogen, fluorine, chlorine, bromine, iodine, cyano, hydroxyl, nitro, OMe, OEt, OPr, OisoPr, OBu, OsecBu, OisoBu, OtertBu, (CH₂)₂OCH₃, O—(CH₂)₃OCH₃, O-cyclopentyl, OCF₃, OCF₂H, OCF₂CF₃, OCF₂CF₂H, OCH₂CH₂N(C₂H₅)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SO-isoPr, SOBuBu, SOsecBu, SO-isoBu, SO-tBu, SO₂Me, SO₂CF₃, SO₂Et, SO₂Pr, SO₂isoPr, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SO₂NHMe, SO₂NMe₂, SO₂NHAc, SO₂NHPh, SO₂NHnBu, SO₂NEt₂, SO₂NHEt, SO₂NPr₂, SO₂NHPr, SO₂NHCF₃, COOH, SO₂NH(CH₂)₃NMe_{2,} SO₂NHCH₂CH═CH₂, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methyl-cyclopropyl)carbonyl]amino, piperazin-1-yl, 4-methylpiperazin-1-yl, morpholin-1-yl, morpholin-4-yl-methyl, NHSO₂CH₃NHSOMe, NHSO₂Me, NHSOEt, NHSO₂Et, NMeSOMeNMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONHCH(C₂H₅)CH₂OCH₃, CONH(CH₂)₂OCH₃, COOH, CO₂CH₃, CO₂Et, CO₂Pr, CO₂isoPr, CO₂Bu, CO₂secBu, CO₂isoBu, CO₂tBu, CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, CHCHF₂OCH₃, C(CH₃)₂OH, CHCH₃OH, CHCHF₂OH, CHCF₃OH, CH₂C(CH₃)₂OCH₃, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, (CH₂)₄OMe, CH₂SMe, (CH₂)₂SMe, (CH₂)₃SMe, (CH₂)₄SMe, CH₂NAc₂, CH₂N(COCF₃)₂, CH₂NHAc, CH₂NHCOCF₃, CH₂NMe₂, (CH₂)₂NHMe, (CH₂)₂NMe₂, (CH₂)₃NMe,₂, (CH₂)₄NHMe, (CH₂)₄NMe₂, CH₂COOCH₃, (CH₂)₂COOMe, (CH₂)₃COOMe, CH₂COOEt, (CH₂)₂COOEt, CH₂COOPr, (CH₂)₂COOPr, CH₂COOisoPr, (CH₂)₂COOisoPr, CH₂COOtertBu, (CH₂)₂COOtertBu, CH₂COO(CH₂)₂OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (pip eridin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2 ,5-dioxo-2,5-dihydro-1 H-pyrrol-1-yl,

if in each case two adjacent radicals R², R^{3e} or R⁴, if appropriate via R¹² or R¹³, form a cycle, the following subunit of the general formula (Ie):

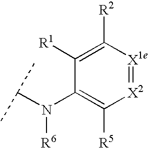

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl- 1, 1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzo furan-5-yl)-amino; and

R^{8e} represents Br and

X², A, R¹, R², R⁴ to R⁷, R^{1-A}, R⁹, R¹⁰, R¹¹, R¹², R¹³ and R¹⁴ have the very particularly preferred meanings given above, and also to agrochemically active salts of these compounds.

Special preference is given to compounds of the formula (Ie) in which one or more of the symbols have one of the especially preferred meanings listed below, i.e.

X^{1e} represents nitrogen or C—R^{3e},

R^{3e} represents hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF₃, OCF₂CF₂H, OCH₂CH₂N(C₂H₅)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO₂Me, SO₂CF₃, SO₂Et, SO₂Pr, SO₂CH₂CH═CH_{2,} SO₂CH₂CN, SO₂CH₂≡CH, SONHMe, SONMe₂, SO₂NHMe, SO₂NMe₂, SO₂NHAc, SO₂NHPh, SO₂NHnBu, SO₂NEt₂, SO₂NHEt, SO₂NPr₂, COOH, SO₂NH(CH₂)₃NMe_{2,} SO₂NHCH₂CH═CH_{2,} COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, morpholin-1-yl, morpholin-4-yl-methyl, NHSO₂CH₃ NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCOMe, OCOEt, OCOPr, OCOisoPr, OCOBu, OCOsecBu, OCOisoBu, OCOtertBu, OSO₂N(CH₃)₂, CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONH(CH₂)₂OCH₃, COOH, CO₂CH₃, CO₂Et, CO₂Pr, CO₂isoPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2 ,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxo-morpholin-4-yl, 2-oxoazetidin-1-yl, 2,5-dioxo-2,5-dihydro-1H-pyrrol-1-yl,

if in each case two adjacent radicals R², R^{3e} or R⁴, if appropriate via R¹² or R¹³, form a cycle, the following subunit of the general formula (Ie):

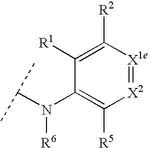

may be (2-oxo-2,3-dihydro-1H-indol-5-yl)amino, 1H-indol-6-ylamino, 1H-indol-5-ylamino, 2-(trifluoromethyl)-1H-b enzimidazol-6-yl]amino, (3-methyl- 1, 1-dioxido-2H-1,2,4-benzothiadiazin-7-yl)amino, (1,1-dioxido-2 H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino; and

R^{8e} represents Br and

X², A, R¹, R², R⁴ to R⁷, R^{1-A}, R⁹, R¹⁰, R¹¹, R¹², R¹³ and R¹⁴ have the especially preferred meanings given above, and also to agrochemically active salts of these compounds.

Preference is furthermore given to compounds of the formula (Ie) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (Ie) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (Ie) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to the agrochemically active salts thereof.

Preference is furthermore given to compounds of the formula (Ie) in which one or more of the symbols have one of the meanings below:

X^{1e} represents nitrogen or CR^{3e},

X² represents nitrogen or CR⁴,

A represents a direct bond, methylene or —CH(CH₃)—,

R¹ to R⁵ and R^{3e} independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (Ie) in which one or more of the symbols have one of the meanings below:

R¹ to R⁵ and R^{3e} independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (Ie) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (Ie) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ or R¹¹ does not represent hydrogen,

or

both radicals R¹¹ simultaneously represent methyl or fluorine,

where the other substituents have one or more of the meanings mentioned above,

and also to agrochemically active salts thereof.

Preference is furthermore given to compounds of the formula (Ie) in which one or more of the symbols have one of the meanings below:

X^{1e} represents CR^{3e} and

X² represents CR⁴,

where the other substituents have one or more of the meanings mentioned above,

and also to the agrochemically active salts thereof.

Preference is furthermore given to compounds of the formula (Ie) in which one or more of the symbols have one of the meanings below:

X^{1e} represents nitrogen and

X² represents nitrogen,

where the other substituents have one or more of the meanings mentioned above,

and also to the agrochemically active salts thereof.

Preference is furthermore given to compounds of the formula (Ie) in which one or more of the symbols have one of the meanings below:

X^{1e} represents CR^{3e} and

X² represents nitrogen,

where the other substituents have one or more of the meanings mentioned above,

and also to the agrochemically active salts thereof.

Preference is furthermore given to compounds of the formula (Ie) in which one or more of the symbols have one of the meanings below:

R¹⁰ represents H or Me,

R¹¹ represents H,

X^{1e} represents CR^{3e} and

X² represents CR⁴,

where the other substituents have one or more of the meanings mentioned above,

and also to the agrochemically active salts thereof.

Preference is furthermore given to compounds of the formula (Ie) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (Ie) in which one or more of the symbols have one of the meanings below:

R¹ represents H,

R⁵ represents H,

where the other substituents have one or more of the meanings mentioned above,

and also to the agrochemically active salts thereof.

Preference is furthermore given to compounds of the formula (Ie) in which one or more of the symbols have one of the meanings below:

R¹ represents H,

R⁵ represents H,

X^{1e} represents CR^{3e} and

X² represents CR⁴,

where the other substituents have one or more of the meanings mentioned above,

and also to the agrochemically active salts thereof.

The radical definitions mentioned above can be combined with one another as desired. Moreover, individual definitions may not apply.

f) Compounds of the formula (If),

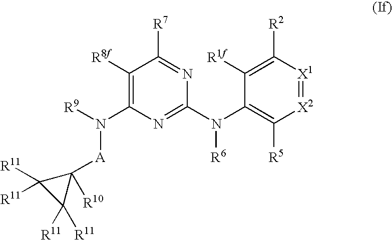

in which the symbols have the following meanings:

R^{1f} represents hydrogen, fluorine, bromine, iodine, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent to one another, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²), (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl; where m=1-8,

where additionally or independently thereof in each case two adjacent radicals R², R³ or R⁴, if appropriate via R¹² or R¹³, together may form a 3 to 7-membered, unsubstituted or substituted, saturated or unsaturated cycle which may contain no or up to four heteroatoms selected from the group consisting of N, O and S, where two oxygen atoms are not adjacent to one another; and

R^{8f} represents methyl and)

X¹, X², A, R², R³, R⁴ to R⁷, R^{1-A}, R⁹, R¹⁰, R¹¹, R¹², R¹³ and R¹⁴ have the general meanings given above, and also to agrochemically active salts of these compounds.

Preference is given to compounds of the formula (If) in which one or more of the symbols have one of the preferred meanings listed below, i.e.

R^{1f} represents hydrogen, fluorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF_{3,} OCF₂CF₂H_{,} OCH₂CF₂H_{,} OCH₂CF_{3,} OCH₂CH₂N(C₂H₅)_{2,} OCH₂CH₂N(CH₃)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SCFH₂, SOMe, SO-Et, SO—Pr, SOisoPr, SOBuBu, SOsecBu, SOisoBu, SO-tBu, SO-pentyl, SOneopentyl, SO₂Me, SO₂CF₃, SO₂Et, SO₂Pr, SO₂isoPr, SO₂BuBu, SO₂secBu, SO₂isoBu, SO₂-tBu, SO₂-pentyl, SO₂neopentyl, SO₂CH₂CH═CH_{2,} SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtertBu, SONHpentyl, SONBu₂, SONHCF_{3,} SON(CF₃)₂, SO₂NHMe, SO₂NMe₂, SO₂NH_{2,} SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe_{2,} SO₂NH(CH₂)₃NEt_{2,} SO₂NHCH₂CH═CH_{2,} COMe, COEt, COPr, COisoPr, COBu, COsecBu, COisoBu, COtertBu, COcyclopropyl, COCHF₂, COCH₂F, COCF₃, NHCOOMe, NHCOOisoPr, NHCOO-tertBu, NHCOOnBuBu, NHCOOpentyl, NHCOOcycloproyl, NH(C═S)OMe, NHCOMe, NHCOCF_{3,} NHCOEt, NHCOPr, NHCOisoPr, NHCOBu, NHCOisoBu, NHCOsecBu, NHCOisoBu, NHCOtertBu, N(Et)COMe, NHCOCH═CH_{2,} NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(CH₃)COCH₃, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, N-sBu₂, N-tBu₂, NHMe, NH₂, NHtertBu, NHsBu, NHEt, NHPr, NHisoPr, NHBu, NHisoBu, NHsecBu, cyclopropylamino, NH(Et)cyclopropyl, NH(Et)cyclopropyl, NHCH(CH₃)CH₂OCH_{3,} NHCH(CH₃)CH₂OEt_{,} NCH₃COCH₃, NEtCOCH₃, acetyl(cyclopropyl)amino, [(1-methylcy clopropyl)carbonyl]amino, piperazin-1-yl, 4-methylpiperazin-1-yl, morpholin-1-yl, morpholin-4-yl-methyl, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tBu, NMeSOtertBu, NMeSO₂tBu, NHSOsBu, NHSO₂sBu, NMeSOsBu, NMeSO₂sBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me)isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)_{2,} OSO₂NEt_{2,} CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH₂, CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, CO₂(CH₂)₂OCH₃, CO₂(CH₂)₂OEt, COCH₂N(CH₃)₂, CO₂(CH₂)₃OCH₃, COCH₂NEt₂, CO₂(CH₂)₃OEt, CH₂SO₂NHMe, CH₂SO₂NHisoPr, CH₂SO₂NHPr, CH₂SO₂NHtertBu, CH₂SO₂NHEt, CH₂SO₂NHisoPr, CH₂SO₂NHPr, CH₂SO₂NHtertBu, CH₂COtertBu, CH₂COCH₃, CH₂COEt, CH₂COOEt, CH₂COOMe, C(CH₃)₂OCH₃, CHCH₃OCH₃, CHCF₃OCH₃, CHCF₃OC₂H₅, C(CH₃)₂OEt, CHCH₃OEt, CHCF₃OEt, C(CH₃)₂OH, CHCH₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₂SH, (CH₂)₃SH, (CH₂)₄SH, CH₂SMe, (CH₂)₂SMe, (CH₂)₃SMe, (CH₂)₄SMe, CH₂SEt, (CH₂)₂SEt, (CH₂)₃SEt, (CH₂)₄SEt, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH₃, CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (If):

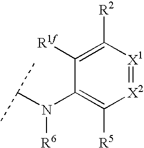

(2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-1-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino; and

R^{8f} represents methyl and

X¹, X², A, R², R³, R⁴ to R⁷, R^{1-A}, R⁹, R¹⁰, R¹¹, R¹², R¹³ and R¹⁴ have the especially preferred meanings given above, and also to agrochemically active salts of these compounds.

Particular preference is furthermore given to compounds of the formula (If) in which one or more of the symbols have one of the particularly preferred meanings below:, i.e.

R^{if} represents hydrogen, fluorine, bromine, iodine, cyano, hydroxyl, nitro, OMe, OEt, OPr, OisoPr, OBu, OsecBu, OisoBu, OtertBu, O—(CH₂)₂OH, O—(CH₂)₂OCH₃, O—(CH₂)₃OH, O—(CH₂)₃OCH₃, O-cyclopentyl, OCF₃, OCF₂H, OCF₂CF₃, OCF₂CF₂H, OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu, ═SO₂Me, SO₂CF₃, SO₂Et, SO₂Pr, SO₂isoPr, SO₂BuBu, SO₂secBu, SO₂isoBu, SO₂-tBu, SO₂CH₂CH═CH₂, SO₂CH₂CN, SO₂CH₂C≡CH, SONHMe, SONMe₂, SONHEt, SONEt₂, SONHPr, SONPr₂, SONHnBu, SONBu₂, SONHCF_{3,} SON(CF₃)₂, SO₂NHMe, SO₂NMe₂, SO₂NH_{2,} SO₂NHAc, SO₂NHPh, SO₂NHnBu, SO₂NEt₂, SO₂NHEt, SO₂NPr₂, SO₂NHPr, SO₂NHCF₃, SO₂N(CF₃)₂, SO₂NH(CH₂)₃NMe₂, SO₂NHCH₂CH═CH_{2,} COMe, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH_{3,} NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, ═N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCOMe, OCOEt, OCOPr, OCOisoPr, OCOBu, OCOsecBu, OCOisoBu, OCOtertBu, OSO₂N(CH₃)₂, CONHEt, CONEt₂, CONHCH₃, CON(CH₃)₂, CONHPr, CONHisoPr, CONHPh, COCH₂CN, CONPr₂, CONHBu, CONHsecBu, CONHisoBu, CONHtertBu, CONHCH₂CH═CH₂, CONHCH(CH₃)CH₂OH, CONHCH(CH₃)CH₂OCH₃, CONHCH(C₂H₅)CH₂OCH₃, CONH(CH₂)₂OCH₃, CONH(CH₂)₂OH, CONH(CH₂)₃OCH₃, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, ═CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, C(CH₃)₂OH, CHCH₃OH, CHCF₃OH, CH₂C(CH₃)₂OCH₃, CHCHF₂OCH₃, CHCHF₂OH, CH₂OH, CH₂NHCOOBn, CH₂NHCOOtertBu, CH₂SO₂CH₃, CH₂NH(CH₂)₂OCH₃, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, ═CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH₃, CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)_{2,} 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)-amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclop entyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,

if in each case two adjacent radicals R², R³ or R⁴, if appropriate via R¹² or R¹³, form a cycle, the following subunit of the general formula (If):

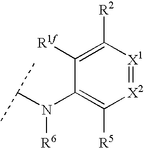

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphony1)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino; and

R^{8f} represents methyl and

X¹, X², A, R², R³, R⁴ to R⁷, R^{1-A}, R⁹, R¹⁰, R¹¹, R¹², R¹³ and R¹⁴ have the particularly preferred meanings given above, and also to agrochemically active salts of these compounds.

Very particular preference is given to compounds of the formula (If) in which one or more of the symbols have one of the very particularly preferred meanings listed below, i.e.

R^{1f} represents hydrogen, fluorine, bromine, iodine, cyano, hydroxyl, nitro, OMe, OEt, OPr, OisoPr, OBu, OsecBu, OisoBu, OtertBu, (CH₂)₂OCH₃, O—(CH₂)₃OCH₃, O-cyclopentyl, OCF₃, OCF₂H, OCF₂CF₃, OCF₂CF₂H, OCH₂CH₂N(C₂H₅)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO-Et, SO—Pr, SOisoPr, SOBuBu, SOsecBu, SOisoBu, SO-tBu, SO₂Me, SO₂CF₃, SO₂Et, SO₂Pr, SO₂isoPr, SO₂BuBu, SO₂secBu, SO₂isoBu, SO₂-tBu, SO₂CH₂CH═CH_{2,} SO₂CH₂CN, SO₂CH₂C≡CH, SONHMe, SONMe₂, SO₂NHMe, SO₂NMe₂, SO₂NH₂, SO₂NHAc, SO₂NHPh, SO₂NHnBu, SO₂NEt₂, SO₂NHEt, SO₂NPr₂, SO₂NHPr, SO₂NHCF₃, COOH, COMe, SO₂NH(CH₂)₃NMe₂, SO₂NHCH₂CH═CH₂, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, NHCO(CH₂)₃OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, piperazin-1-yl, 4-methylpiperazin-1-yl, morpholin-1-yl, morpholin-4-yl-methyl, NHSO₂CH₃ NHSOMe, NHSO₂Me, NHSOEt, NHSO₂Et, NMeSOMeNMeSO₂Me, NMeSOEt, NMeSO₂Et, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONHCH(C₂H₅)CH₂OCH₃, CONH(CH₂)₂OCH₃, COOH, CO₂CH₃, CO₂Et, CO₂Pr, CO₂isoPr, CO₂Bu, CO₂secBu, CO₂isoBu, CO₂tBu, CO₂(CH₂)₂OCH₃, COCH₂N(CH₃)₂, CO₂(CH₂)₃OCH₃, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCH₃OCH₃, CHCF₃OCH₃, CHCF₃OC₂H₅, CHCHF₂OCH₃, C(CH₃)₂OH, CHCH₃OH, CHCHF₂OH, CHCF₃OH, CH₂C(CH₃)₂OCH₃, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, (CH₂)₄OMe, CH₂SMe, (CH₂)₂SMe, (CH₂)₃SMe, (CH₂)₄SMe, CH₂NAc₂, CH₂N(COCF₃)₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, CH₂COOPr, (CH₂)₂COOPr, CH₂COOisoPr, (CH₂)₂COOisoPr, CH₂COOtertBu, (CH₂)₂COOtertBu, CH₂COO(CH₂)₂OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, 1-methoxycyclopropyl, 1-chlorocyclopropyl, cyclobutyl, cyclopentyl, cyclohexyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (pip eridin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxo-morpholin-4-yl, 2-oxoazetidin-1-yl, 2 ,5-dioxo-2,5-dihydro-1H-pyrrol-1-yl,

if in each case two adjacent radicals R², R³ or R⁴, if appropriate via R¹² or R¹³, form a cycle, the following subunit of the general formula (If):

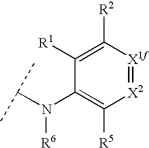

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzo furan-5-yl)-amino; and

R^{8f} represents methyl and

X¹, X², A, R² to R⁷, R^{1-A}, R⁹, R¹⁰, R¹¹, R¹², R¹³ and R¹⁴ have the very particularly preferred meanings given above, and also to agrochemically active salts of these compounds.

Special preference is given to compounds of the formula (If) in which one or more of the symbols have one of the especially preferred meanings listed below, i.e.

R^{1f} represents hydrogen, fluorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF_{3,} OCF₂CF₂H, OCH₂CH₂N(C₂H₅)_{2,} OCH(CH₃)CH₂OCH_{3,} SH, SMe, SPh, SEt, SPr, SisoPr, SBu, SsecBu, SisoBu, StBu, Spentyl, Ssecpentyl, Sneopentyl, SOctyl, SCF₃, SCF₂H, SOMe, SO₂Me, SO₂CF₃, SO₂Et, SO₂Pr, SO₂CH₂CH═CH₂, SO₂CH₂CN, SO₂CH₂C≡CH, SONHMe, SONMe₂, SO₂NHMe, SO₂NMe₂, SO₂NH₂, SO₂NHAc, SO₂NHPh, SO₂NHnBu, SO₂NEt₂, SO₂NHEt, SO₂NPr₂, COOH, COMe, SO₂NH(CH₂)₃NMe₂, SO₂NHCH₂CH═CH₂, COEt, COPr, COisoPr, COBu, COsecBu, COisoBu, COtertBu, COCHF₂, COCF₃, NHCOOMe, NHCOOisoPr, NHCOOtertBu, NHCOOnBuBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, morpholin-1-yl, morpholin-4-yl-methyl, NHSO₂CH₃NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCOMe, OCOEt, OCOPr, OCOisoPr, OCOBu, OCOsecBu, OCOisoBu, OCOtertBu, OSO₂N(CH₃)_{2,} CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONH(CH₂)₂OCH₃, COOH, CO₂CH₃, CO₂Et, CO₂Pr, CO₂iPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂OMe, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol-2-yl-, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2 ,5-dioxo-2,5-dihydro-1H-pyrrol-1-yl,

if in each case two adjacent radicals R², R³ or R⁴, if appropriate via R¹² or R¹³, form a cycle, the following subunit of the general formula (If):

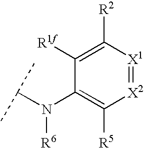

may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2 H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)-amino; and

R^{8f} represents methyl and

X², A, R², R³, R⁴ to R⁷, R^{1-A}, R⁹, R¹⁰, R¹¹, R¹², R¹³ and R¹⁴ have the especially preferred meanings given above, and also to agrochemically active salts of these compounds.

Preference is furthermore given to compounds of the formula (If) in which one or more of the symbols have one of the meanings below:

A represents a direct bond, methylene or —CH(CH₃)—,

R¹⁰ represents H, OEt,

R¹¹ represents H,

where the other substituents have one or more of the meanings mentioned above,

and also to the agrochemically active salts thereof.

Preference is furthermore given to compounds of the formula (If) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where the other substituents have one or more of the meanings mentioned above,

and also to the agrochemically active salts thereof.

Preference is furthermore given to compounds of the formula (If) in which one or more of the symbols have one of the meanings below:

A represents a direct bond, methylene or —CH(CH₃)—,

R¹⁰ represents H, OEt, CO₂Et, 2-Cl-phenyl, phenyl, CH₂OPh,

R¹¹ represents H, F, Cl, Me,

where in each case only one of the radicals R¹⁰ and R¹¹ does not represent hydrogen,

or

both R¹¹ simultaneously represent methyl or fluorine,

where the other substituents have one or more of the meanings mentioned above,

and also to the agrochemically active salts thereof.

Preference is furthermore given to compounds of the formula (If) in which one or more of the symbols have one of the meanings below:

X¹ represents nitrogen or CR³,

X² represents nitrogen or CR⁴,

A represents a direct bond, methylene or —CH(CH₃)—,

R² to R⁵ and R^{1f} independently of one another represent COCl, CH═NOR¹², CR¹³═NOR¹², SF₅,

where the other substituents have one or more of the meanings mentioned above,

and also to agrochemically active salts thereof.

Preference is furthermore given to compounds of the formula (If) in which one or more of the symbols have one of the meanings below:

R² to R⁵ and R^{1f} independently of one another represent CH═NOMe, C(CH₃)═NOMe, CH═NOEt, C(CH₃)═NOEt,

where the other substituents have one or more of the meanings mentioned above,

and also to agrochemically active salts thereof.

Preference is furthermore given to compounds of the formula (If) in which one or more of the symbols have one of the meanings below:

A represents a direct bond, methylene or —CH(CH₃)—,

R¹⁰ represents CH₂OPh,

where the other substituents have one or more of the meanings mentioned above,

and also to agrochemically active salts thereof.

Preference is furthermore given to compounds of the formula (If) in which one or more of the symbols have one of the meanings below:

A represents a direct bond, methylene or —CH(CH₃)—,

R¹⁰ represents hydrogen, Me, CF₃, OEt, COOH, COOMe, COOEt, phenyl, benzyl, 2-chlorophenyl, 3-fluorophenyl, 3-bromophenyl, 3-trifluoromethylphenyl, 3-chlorophenyl, 3-methoxyphenyl, 4-methoxyphenyl, CH₂OPh,

R¹¹ represents identical or different hydrogen, fluorine, methyl, CF₃, phenyl, OEt, SMe, 4-fluorophenyl, 4-methylphenyl,

where in each case only one of the radicals R¹⁰ and R¹¹ does not represent hydrogen,

or

both R¹¹ simultaneously represent methyl or fluorine,

where the other substituents have one or more of the meanings mentioned above,

and also to agrochemically active salts thereof.

Preference is furthermore given to compounds of the formula (If) in which one or more of the symbols have one of the meanings below:

X¹ represents CR³ and

X² represents CR⁴,

where the other substituents have one or more of the meanings mentioned above,

and also to the agrochemically active salts thereof.

Preference is furthermore given to compounds of the formula (If) in which one or more of the symbols have one of the meanings below:

X¹ represents nitrogen and

X² represents nitrogen,

where the other substituents have one or more of the meanings mentioned above,

and also to the agrochemically active salts thereof.

Preference is furthermore given to compounds of the formula (If) in which one or more of the symbols have one of the meanings below:

X¹ represents CR³ and

X² represents nitrogen,

where the other substituents have one or more of the meanings mentioned above,

and also to the agrochemically active salts thereof.

Preference is furthermore given to compounds of the formula (If) in which one or more of the symbols have one of the meanings below:

R¹⁰ represents H or Me,

R¹¹ represents H,

X¹ represents CR³ and

X² represents CR⁴,

where the other substituents have one or more of the meanings mentioned above,

and also to the agrochemically active salts thereof.

Preference is furthermore given to compounds of the formula (If) in which one or more of the symbols have one of the meanings below:

R⁶ represents H, CHO, COCH₃, COCF₃

R⁷ represents H,

R⁹ represents H, Me, CHO, COCH₃

where the other substituents have one or more of the meanings mentioned above,

and also to the agrochemically active salts thereof.

Preference is furthermore given to compounds of the formula (If) in which one or more of the symbols have one of the meanings below:

R^{1f} represents H,

R⁵ represents H,

where the other substituents have one or more of the meanings mentioned above,

and also to the agrochemically active salts thereof.

Preference is furthermore given to compounds of the formula (If) in which one or more of the symbols have one of the meanings below:

R^{1f} represents H,

R⁵ represents H,

X¹ represents CR³ and

X² represents CR⁴,

where the other substituents have one or more of the meanings mentioned above,

and also to the agrochemically active salts thereof.

The radical definitions mentioned above can be combined with one another as desired. Moreover, individual definitions may not apply.

The compounds of the formula (Ia), (Ib), (Ic), (Id), (Ie) and (If) can be present either in pure form or as mixtures of various possible isomeric forms, in particular of stereoisomers, such as, for example, E and Z, threo and erythro, and also optical isomers, such as R and S isomers or atropisomers, and, if appropriate, also of tautomers. What is claimed are both the E and the Z isomers and the threo and erythro and also the optical isomers, any mixtures of these isomers, and also the possible tautomeric forms.

Depending on the nature of the substituents defined above, the compounds of the formulae (I), (Ia), (Ib), (Ic), (Id), (Ie) and (If) have acidic or basic properties and may form salts, if appropriate also inner salts, or adducts with inorganic or organic acids or with bases or with metal ions. If the compounds of the formulae (I), (Ia), (Ib), (Ic), (Id), (Ie) and (If) carry amino, alkylamino or other groups which induce basic properties, these compounds can be converted with acids into salts, or they are obtained directly as salts in the synthesis.

Examples of inorganic acids are hydrohalic acids, such as hydrofluoric acid, hydrochloric acid, hydrobromic acid and hydroiodic acid, sulphuric acid, phosphoric acid and nitric acid, and acidic salts, such as NaHSO₄ and KHSO₄.

Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or -disulphonic acids (aromatic radicals, such as phenyl and naphthyl which carry one or two sulphonic acid groups), alkylphosphonic acids (phosphonic acids having straight-chain or branched alkyl radicals of 1 to 20 carbon atoms), arylphosphonic acids or -diphosphonic acids (aromatic radicals, such as phenyl and naphthyl, which carry one or two phosphonic acid radicals), where the alkyl radicals and aryl radicals may carry further substituents, for example p-toluenesulphonic acid, salicylic acid, p-aminosalicylic acid, 2-phenoxybenzoic acid, 2-acetoxybenzoic acid, etc.

The salts which can be obtained in this manner also have fungicidal properties.

The radical definitions mentioned above can be combined with one another as desired. Moreover, individual definitions may not apply.

In the definitions of the symbols given in the formulae above, collective terms were used which are generally representative for the following substituents:

halogen: fluorine, chlorine, bromine and iodine;

alkyl: saturated straight-chain or branched hydrocarbon radicals having 1 to 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 8 carbon atoms (as mentioned above), where in these groups some or all of the hydrogen atoms may be replaced by halogen atoms as mentioned above, for example C₁-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;

alkenyl: unsaturated straight-chain or branched hydrocarbon radicals having 2 to 8 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl- 1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl- 1-pentenyl, 3-methyl- 1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-penlynyl, 3-penlynyl, 4-penlynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: monocyclic saturated hydrocarbon groups having 3 to 8 carbon ring members, such as cyclopropyl, cyclobutyl, cyclopentyl, cyclohexyl,

cycloalkenyl: monocyclic nonaromatic hydrocarbon groups having 3 to 8 carbon ring members and at least one double bond, such as cyclopenten-1-yl, cyclohexen-1-yl, cyclohepta-1,3-dien-1-yl;

alkoxycarbonyl: an alkoxy group having 1 to 6 carbon atoms (as mentioned above) which is attached to the skeleton via a carbonyl group (—CO—);

oxyalkyleneoxy: divalent straight-chain chains of 1 to 3 CH₂ groups where both valencies are attached to the skeleton via an oxygen atom, for example OCH₂O, OCH₂CH₂O and OCH₂CH₂CH₂O;

a five- to ten-membered saturated or partially unsaturated heterocycle which contains one to four heteroatoms from the group consisting of oxygen, nitrogen and sulphur: mono- or bicyclic heterocycles (heterocyclyl) which contain, in addition to carbon ring members, one to three nitrogen atoms and/or one oxygen or sulphur atom or one or two oxygen and/or sulphur atoms; if the ring contains a plurality of oxygen atoms, these are not directly adjacent; for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;

a five- to ten-membered aromatic heterocycle which contains one to four heteroatoms from the group consisting of oxygen, nitrogen and sulphur: mono- or bicyclic heteroaryl, for example

-5-membered heteroaryl which contains one to four nitrogen atoms or one to three nigrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

-benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members and in which two adjacent carbon ring members or one nitrogen and one adjacent carbon ring member may be bridged by a buta-1,3-diene-1,4-diyl group, in which one or two carbon atoms may be replaced by nitrogen atoms;

-5-membered heteroaryl which is attached via nitrogen and contains one to four nitrogen atoms, or benzo-fused 5-membered heteroaryl which is attached via nitrogen and contains one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;

-6-membered heteroaryl which contains one to three or one to four nitrogen atoms: 6-membered heteroaryl groups which, in addition to carbon atoms, may contain one to three or one to four nitrogen atoms as ring members, for example 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The present invention furthermore relates to a process for preparing the diaminopyrimidines of the formulae (Ia), (Ib), (Ic), (Id), (Ie) and (If) according to the invention, comprising at least one of the following steps (a) to (e):
(a) Reaction of 2,4 dihalopyrimidinens of the formula (III) with cyclopropylamines of the formula (II) in the presence of a base, if appropriate in the presence of a solvent, if appropriate in the presence of a catalyst, to give compounds of the formula (V), according to the reaction scheme below (Scheme 1):

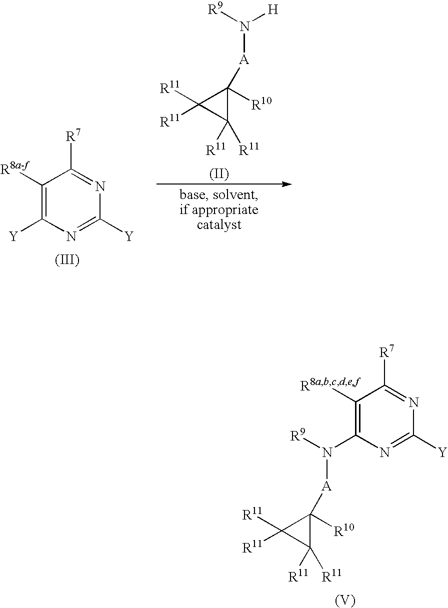

where Y═F, Cl, Br, I
(b) Reaction of compounds of the formula (V) with aromatic amines of the formula (IV), if appropriate in the presence of an acid, if appropriate in the presence of a solvent, according to the reaction scheme below (Scheme 2):

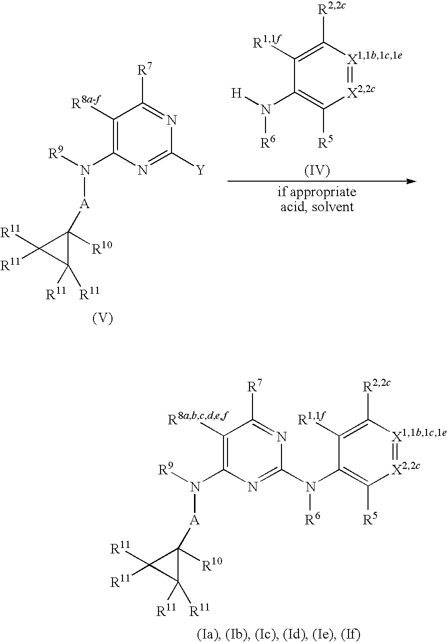

where Y═F, Cl, Br, I
(c) Reaction of compounds of the formula (VIb) with an aromatic amine of the formula (IV), if appropriate in the presence of an acid and of a solvent, according to the reaction scheme below (Scheme 3):

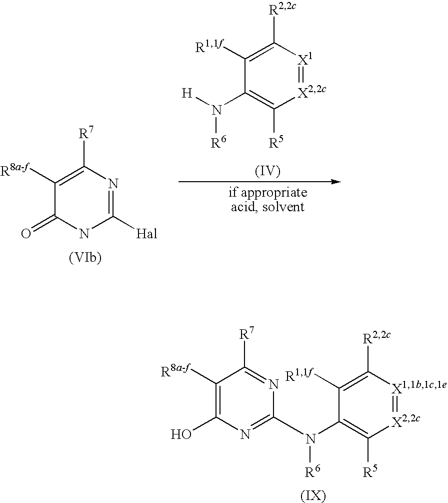

where Hal=F, Cl, Br, I
(d) Reaction of compounds of the formula (IX) with a halogenating agent, if appropriate in the presence of a solvent, to give compounds of the formula (X), according to the reaction scheme below (Scheme 4):

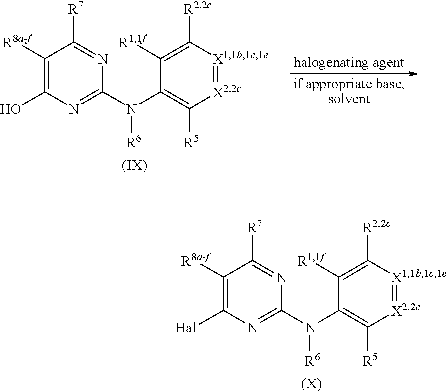

where Hal=F, Cl, Br, I
(e) Reaction of compounds of the formula (X) with cyclopropylamines of the formula (II) in the presence of a base, if appropriate in the presence a solvent, if appropriate in the presence a catalyst, to give compounds of the formulae (Ia), (Ib), (Ic), (Id), (Ie) and (If), according to the reaction scheme below (Scheme 5):

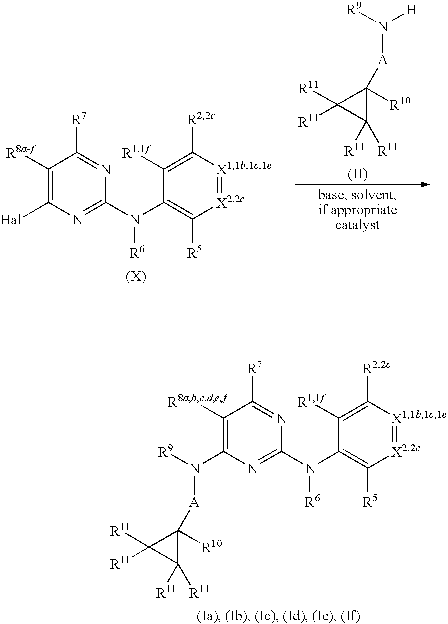

where the definitions of the radicals R¹ to R¹¹ and X¹ and X² in the schemes above correspond to the definitions given above and Hal=F, Cl, Br, I.

One option of preparing the intermediate (V) is shown in Scheme 1.

The amino compounds of the formula (II) are either commercially available, or they can be prepared according to literature methods (described, for example, in J. Org. Chem. 2002, 67, 3965; J. Med. Chem. 2004, 47, 5860). A further method for preparing suitable amino compounds (II) is, for example, the rearrangement of suitable carboxylic acid derivatives to the corresponding amino compounds (described, for example, in J. Am. Chem. Soc. 1953, 75, 1382-1384).

Suitable substituted 2,4-dihalopyrimidinens (III) are either commercially available or can be prepared according to literature procedures, for example from commercially available substituted uracils (for example R^{8b}═CN: J. Org. Chem. 1962, 27, 2264; J. Chem. Soc. 1955, 1834; Chem. Ber. 1909, 42, 734; R^{8d}═CF₃: J. Fluorine Chem. 1996, 77, 93; see also WO 2000/047539).

Initially, using a suitable base at a temperature of from −30° C. to +80° C. in a suitable solvent, such as for example, dioxane, THF, dimethylformamide or acetonitrile, a cyclopropylamine (II) is reacted with a 2,4-dihalopyrimidine (III) over a period of 1-24 h. Suitable for use as bases are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst, such as, for example, palladium, together with a suitable ligand, such as, for example triphenylphosphine or xanthphos.

Some of the compounds of the formula (V) are novel and thus also form part of the subject-matter of the present invention.

Novel are compounds of the formula (Va) in which

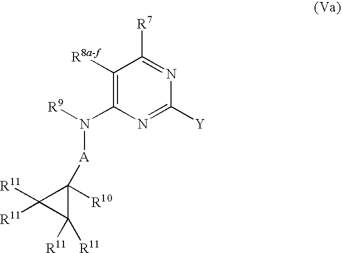

A, R⁷, R^{8d}, R⁹, R¹⁰, R¹¹ and R¹⁴ have the general, preferred, particularly preferred and very particularly preferred meanings as defined above,

Y═F, Cl, Br or I,

where

R^{8a} represents chlorine, iodine, CFH₂, CF₂H or CCl₃,

with the proviso that, if

R^{8a} and Y represent chlorine and A represents a direct bond,

R¹⁰ must not represent CO₂H or CO₂Me,

R^{8b} represents cyano with the proviso that, if Y represents chlorine and A represents a direct bond, R¹⁰ and R¹¹ must not simultaneously represent hydrogen,

R^{8c} represents fluorine with the proviso that, if Y represents chlorine and A represents a direct bond, R¹⁰ and R¹¹ must not simultaneously represent hydrogen,

R^{8e} represents Br

with the proviso that, if Y represents chlorine and A represents methylene or a direct bond, R¹⁰ and R¹¹ must not simultaneously represent hydrogen,

R^{8f} represents methyl with the proviso that, if Y represents chlorine and A represents methylene or a direct bond steht, R⁹ must not represent nPr. One possibility of how to prepare the compounds (Ia), (Ib), (Ic), (Id), (Ie) and (If) is shown in Scheme 2.

The substituted aromatic amines (IV) are either commercially available or can be prepared from commercially available precursors by methods known from the literature. Aromatic amines which carry one or more identical or different substituents in the aromatic moiety can be prepared by a large number of methods described in the relevant literature. Hereinbelow, some of the methods are mentioned by way of example.

Sulphonamide- or sulphonic ester-substituted arylamines can be prepared, for example, by the reaction, known from the literature, of commercially available aminosulphonic acids with chlorinating agents (for example POCl₃) and subsequent reaction of the sulphochlorides formed with O- or N-nucleophiles.

The customary methods for preparing N-monoacylated diaminoaromatic compounds are illustrated below. Thus, for example, nitroanilines can be converted by standard methods with acyl halides, chloroformic esters or iso(thio)cyanates into the corresponding N-acylnitroaromatic compounds which can then be reduced by processes known from the literature to N-acylaminoaromatic compounds. A further method describes the preparation of the compounds mentioned via transition metal-catalyzed cross-coupling of aminohaloaromatic compounds and N-acyl compounds (see, for example, J. Am. Chem. Soc. 2001, 123, 7727).

### Synthesis of N-monoacylated diaminoaromatic Compounds

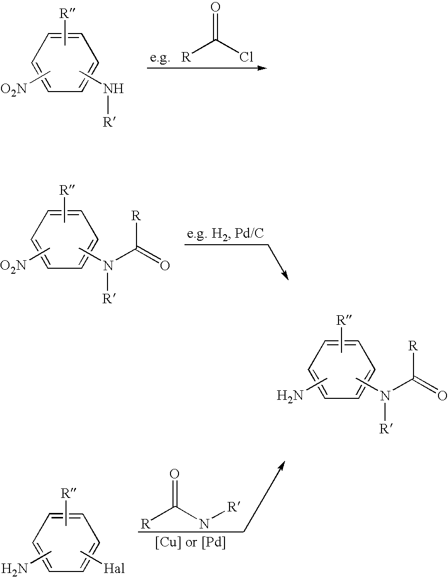

Cyclic radicals R¹ to R⁵ attached via N can be prepared, for example, by condensation of nitroaminoaromatic compounds with haloalkylcarbonyl halides or diesters or diester equivalents or lactones; the subsequent reduction of the nitro group affords the desired aromatic amine. A further possibility of synthesizing of radicals R¹ to R⁵ attached via N is the condensation of nitroarylhydrazines with diesters or diester equivalents, propargyl acid esters or ketoesters. The reduction of the nitro group affords the aniline.

The intermediate (V) is reacted in the presence of Brönstedt acids such as, for example anhydrous hydrochloric acid, camphorsulphonic acid or p-toluenesulphonic acid in a suitable solvent such as, for example dioxane, THF, DMSO, DME, 2-methoxyethanol, n-butanol or acetonitrile at a temperature of 0° C.-140° C. over a period of 1-48 h with an aromatic amine (IV). Analogously described, for example, in Bioorg. Med. Chem. Lett. 2006, 16, 2689; GB2002 A1-2369359, Org. Lett. 2005, 7, 4113).

Alternatively, the conversion of (V) and (IV) into (Ia), (Ib), (Ic), (Id), (Ie) and (If) can also be carried out under base catalysis, i.e. using, for example, carbonates, such as potassium carbonate, alkoxides, such as potassium tert-butoxide, or hydrides, such as sodium hydride, where the catalytic use of a transition metal such as, for example, palladium together with a suitable ligand such as, for example, xanthphos may also be of use.

Finally there is also the option to carry out the conversion of (V) and (IV) into (Ia), (Ib), (Ic), (Id), (Ie) and (If) in the absence of solvents and/or Brönstedt acids (described, for example, in Bioorg. Med. Chem. Lett. 2006, 16, 108; Bioorg. Med. Chem. Lett. 2005, 15, 3881).

One possibility of preparing compounds of the formula (IX) and also (IXa) is shown in Scheme 3.

2-halo-substituted pyrimidin-4-ones (VIb) are obtainable from 2,4-dihalo-substituted pyrimidines by regioselective hydrolysis. This is described, for example, in Russ. J. Org. Chem. 2006, 42, 580; J. Med. Chem.1965, 8, 253.

Intermediates of the formula (VIb) are reacted in the presence of Brönstedt acids such as, for example anhydrous hydrochloric acid, camphorsulphonic acid or p-toluenesulphonic acid in a suitable solvent such as, for example dioxane, THF, DMSO, DME, 2-methoxyethanol, n-butanol or acetonitrile at a temperature of 0° C.-140° C. over a period of 1-48 h with an aromatic amine (IV).

Alternatively, the conversion (VIb) and (IV) into (IX) can also be carried out under base catalysis, i.e. using, for example, carbonates, such as potassium carbonate, alkoxides, such as potassium tert-butoxide, or hydrides, such as sodium hydride, where the catalytic use of a transition metal such as, for example, palladium together with a suitable ligand such as, for example, xanthphos may also be of use.

Finally there is also the option to carry out the conversion of (VIb) and (IV) into (IX) in the absence of solvents and/or Brönstedt acids (described, for example, in Bioorg. Med. Chem. Lett. 2006, 16, 108; Bioorg. Med. Chem. Lett. 2005, 15, 3881).

Some of the compounds of the formula (IX) are novel and thus also form part of the subject-matter of the present invention.

Novel are compounds of the formula (IXa),

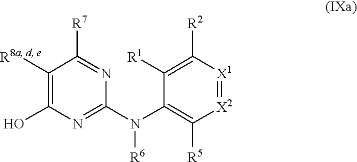

in which the symbols have the following meanings:

X¹, X², R¹ to R⁵, R^{8d,e}, R¹² and R¹³ have the general, preferred, particularly preferred, very particularly preferred and especially preferred meanings given above,

R⁶ and R⁷ represent hydrogen,

R^{8a} represents chlorine, iodine, CFH₂, CF₂H, CCl₃,

with the proviso that, if

R¹═R²═R⁵═H and X²═CH or N,

R³ must not represent H, CO₂H, (CH₂)₂OH, SMe, SOMe, SO₂NH₂ or cyano

or

with the proviso that, if

R¹═R⁵═H and X¹═CH,

neither R² nor R⁴ must represent OH or CONH₂.

One possibility of how to prepare compounds of the formula (X) and also (Xa) is shown in Scheme 4.

Intermediates of the formula (IX) can be converted by reaction with suitable halogenating agents such as, for example, thionyl chloride, phosphorus pentoxide or phosphoryl chloride or a mixture thereof, if appropriate in the presence of a suitable solvent such as, for example, toluene or ethanol and if appropriate in the presence of a suitable base such as, for example, triethylamine into 2-anilino-4-chloropyrimidines of the formula (X). Analogously described, for example, in J. Med. Chem. 1989, 32, 1667; J. Heterocycl. Chem. 1989, 26, 313.

Some of the compounds of the formula (X) are novel and thus also form part of the subject-matter of the present invention.

Novel are compounds of the formula (Xa),

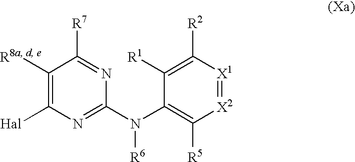

in which the symbols have the following meanings:

X¹, X², R² to R⁴, R⁷, R^{8d}, R^{8e}, R¹² and R¹³ have the general, preferred, particularly preferred, very particularly preferred and especially preferred meanings given above, and

Hal represents fluorine, chlorine, bromine or iodine,

R^{8a} represents chlorine, iodine, CFH₂, CF₂H, CCl₃ and cyano,

R¹, R⁵ and R⁶ represent hydrogen

with the proviso that, if

X²═CH or N and X¹═CR³,

R³ does not represent CON(Me)-4-(N-methylpiperidinyl), N-piperazinyl, CO-1-(4-methylpiperazinyl), N-morpholinyl, SO₂Me, CONH₂, Me, OMe, COO-benzyl, COOH, COCl, CN, SO₂NH₂, NO₂, NMe₂ or Cl,

or

with the proviso that, if

X¹═CH and X²═CR⁴,

R² or R⁴ does not represent CN, Cl or 5-oxazolyl,

or

with the proviso that, if

X¹═CR³ and X²═CR⁴,

R², R³ and R⁴ do not represent chlorine,

or

with the proviso that, if

X¹═CR³, X²═CR⁴ and R^{8d}═CF₃,

R² and R³ or R³ and R⁴ together do not form a saturated or partially unsaturated heterocycle.

A further possibility of how to prepare the compounds (Ia), (Ib), (Ic), (Id), (Ie) and (If) is shown in Scheme 5.

To prepare compounds of the formulae (Ia), (Ib), (Ic), (Id), (Ie) and (If), the intermediate (X) is reacted in the presence of bases such as, for example, carbonates such as potassium carbonate, alkoxides, such as potassium tert-butoxide, or hydrides, such as sodium hydrid, in a suitable solvent such as, for example dioxane, THF, DMSO, DME, 2-methoxyethanol, n-butanol or acetonitrile at a temperature of 0° C.-140° C. over a period of 1-48 h with cyclopropylamines of the formula (II), where the catalytic use of a transition metal such as, for example, palladium, together with a suitable ligand, such as, for example triphenylphosphine or xanthphos may also be of use.

In general, it is also possible to chose another route for preparing the compounds (Ia), (Ib), (Ic), (Id), (Ie) and (If) according to the invention, as shown in Scheme 6.

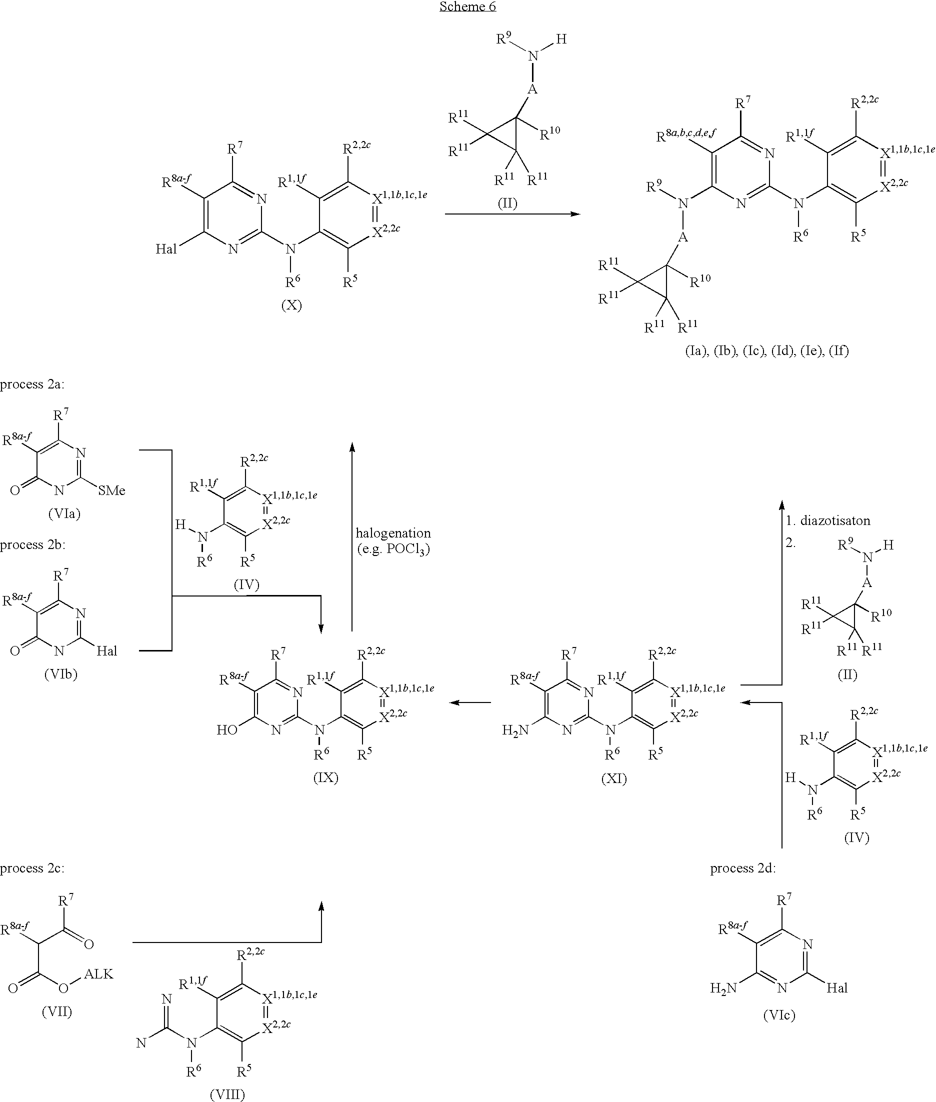

A further method for preparing diaminopyrimidines of the formulae (Ia), (Ib), (Ic), (Id), (Ie) and (If) is shown in Scheme 7:

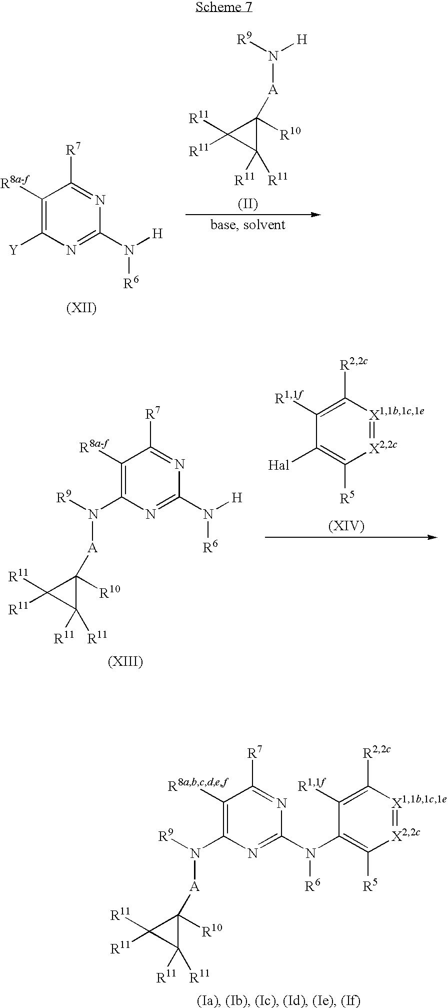

Starting with 4-halo-substituted 2-amino pyrimidines (XII), which can be obtained, for example, analogously to (X) from compounds of the type (VIa), (VIb) or (VII) by reaction with R⁶-amines and subsequent chlorination in the 4-position, it is possible to obtain, after addition of an amino compound (II), certain diaminopyrimidines (XIII). In a subsequent transition metal-catalysed step, these compounds can be reacted with an aryl halide (XIV) (as described, for example, in Org. Lett. 2002, 4, 3481) to give the desired target compound (Ia), (Ib), (Ic).

The processes according to the invention for preparing the compounds of the formulae (Ia), (Ib), (Ic), (Id), (Ie) and (If) are preferably carried out using one or more reaction auxiliaries.

Suitable reaction auxiliaries are, if appropriate, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl -2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroine, benzene, toluene, xylene, methylene chloride, ethylene chloride, chloroform, carbon tetrachloride, chlorobenzene and o-dichlorbenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles such as, for example, acetonitrile or propionitrile, amides such as, for example, dimethylformamide, dimethylacetamide and N-methylpyrrolidone, and also dimethyl sulphoxide, tetramethylene sulphone and hexamethyl-phosphoric triamide and DMPU.

The reaction temperatures in the processes according to the invention can be varied within a relatively large range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the particular starting materials required are generally employed in approximately equimolar amounts. However, it is also possible to use a relatively large excess of one of the components employed in each case. Work-up in the processes according to the invention is in each case carried out by customary methods (cf. the Preparation Examples).

Suitable reaction auxiliaries are, if appropriate, the customary inorganic or organic Brönstedt or Lewis acids. These preferably include hydrohalic acids or mineral acids, such as, for example, hydrochloric acid or hydrobromic acid, sulphuric acid, sulphurous acid, nitric acid, nitrous acid, phosphoric acid, Lewis acids, such as, for example, aluminium trichloride, boron trifluoride, boron trichloride, boron tribromide, titanium tetrachloride, tin tetrachloride, cerium trichloride, (transition) metal triflates, such as, for example, trialkylsilyl triflates, copper triflate, ytterbium triflate, imides, such as, for example, trifluoromethanesulphonimide, sulphonic acids, such as, for example, methanesulphonic acid, trifluoromethanesulphonic acid, p-toluenesulphonic acid, camphorsulphonic acid, carboxylic acids, such as, for example, formic acid, acetic acid, trifluoroacetic acid, propionic acid, oxalic acid, benzoic acid.

In general, compounds of the formula (I) can be prepared, for example, by sequential nucleophilic addition of an aliphatic amine (II) and a (hetero)aromatic amine (IV) to a suitable substituted pyrimidine (III), as illustrated below in Scheme 8:

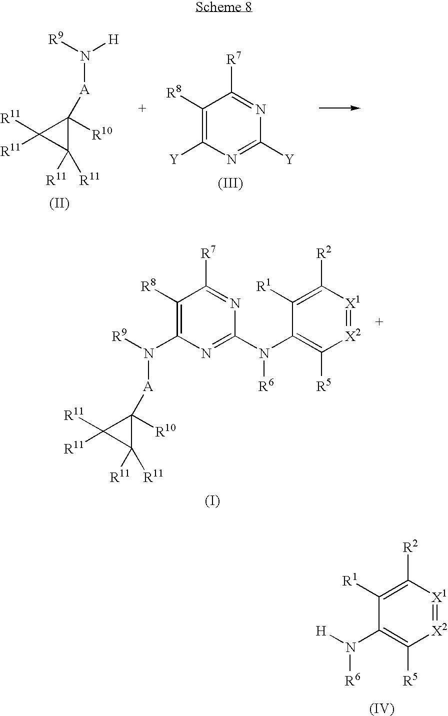

Here, Y represents in each case independently of the other a suitable leaving group, for example a halogen atom (Hal=F, Cl, Br, I), SMe, SO₂Me, SOMe or else triflate (CF₃SO₂O: for pyrimidines known from WO2005095386).

The synthesis of diaminopyrimidines of the formula (I) according to Scheme 8 or else via other routes has been described in the literature in numerous instances (see also, for example, U.S. Ser. No. 04/256,145, WO 05/016893, WO 05/013996, WO 04/056786, WO 04/056807, WO 03/076437, WO 02/096888, WO 02/004429)

The compounds according to the invention exhibit a potent microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

The diaminopyrimidines according to the invention have very good fungicidal properties and can be used for controlling phytopathogenic fungi, such as Plasmodiophoromycetes, Oomycetes, Chytridiomycetes, Zygomycetes, Ascomycetes, Basidiomycetes and Deuteromycetes etc.

Examples which may be mentioned, but not by limitation, of some pathogens of fungal and bacterial diseases which come under the abovementioned general terms are:

diseases caused by powdery mildew pathogens, such as, for example Blumeria species such as, for example, Blumeria graminis; Podosphaera species such as, for example, Podosphaera leucotricha; Sphaerotheca species such as, for example, Sphaerotheca fuliginea; Uncinula species such as, for example, Uncinula necator;

diseases caused by rust pathogens such as, for example, Gymnosporangium species such as, for example, Gymnosporangium sabinae; Hemileia species such as, for example, Hemileia vastatrix; Phakopsora species such as, for example, Phakopsora pachyrhizi and Phakopsora meibomiae; Puccinia species such as, for example, Puccinia recondita or Puccinia graminis; Uromyces species such as, for example, Uromyces appendiculatus;

diseases caused by pathogens from the Oomycetes group such as, for example, Bremia species such as, for example, Bremia lactucae; Peronospora species such as, for example, Peronospora pisi or P. brassicae; Phytophthora species such as, for example, Phytophthora infestans; Plasmopara species such as, for example, Plasmopara viticola; Pseudoperonospora species such as, for example, Pseudoperonospora humuli or Pseudoperonospora cubensis; Pythium species such as, for example, Pythium ultimum;

leaf spot diseases and leaf wilts caused by, for example, Alternaria species such as, for example, Alternaria solani; Cercospora species such as, for example, Cercospora beticola; Cladosporium species such as, for example, Cladosporium cucumerinum; Cochliobolus species such as, for example, Cochliobolus sativus (conidial form: Drechslera, Syn: Helminthosporium); Colletotrichum species such as, for example, Colletotrichum lindemuthanium; Cycloconium species such as, for example, Cycloconium oleaginum; Diaporthe species such as, for example, Diaporthe citri; Elsinoe species such as, for example, Elsinoe fawcettii; Gloeosporium species such as, for example, Gloeosporium laeticolor; Glomerella species such as, for example, Glomerella cingulata; Guignardia species such as, for example, Guignardia bidwelli; Leptosphaeria species such as, for example, Leptosphaeria maculans; Magnaporthe species such as, for example, Magnaporthe grisea; Mycosphaerella species such as, for example, Mycosphaerella graminicola and Mycosphaerella fijiensis; Phaeosphaeria species such as, for example, Phaeosphaeria nodorum; Pyrenophora species such as, for example, Pyrenophora teres; Ramularia species such as, for example, Ramularia collo-cygni; Rhynchosporium species such as, for example, Rhynchosporium secalis; Septoria species such as, for example, Septoria apii; Typhula species such as, for example, Typhula incarnata; Venturia species such as, for example, Venturia inaequalis;

root and stem diseases caused by, for example, Corticium species such as, for example, Corticium graminearum; Fusarium species such as, for example, Fusarium oxysporum; Gaeumannomyces species such as, for example, Gaeumannomyces graminis; Rhizoctonia species such as, for example, Rhizoctonia solani; Tapesia species such as, for example, Tapesia acuformis or Tapesia yallundae; Thielaviopsis species such as, for example, Thielaviopsis basicola;

ear and panicle diseases (including maize cobs), caused by, for example, Alternaria species such as, for example, Alternaria spp.; Aspergillus species such as, for example, Aspergillus flavus; Cladosporium species such as, for example, Cladosporium cladosporioides; Claviceps species such as, for example, Claviceps purpurea; Fusarium species such as, for example, Fusarium culmorum; Gibberella species such as, for example, Gibberella zeae; Monographella species such as, for example, Monographella nivalis;

diseases caused by smuts such as, for example, Sphacelotheca species such as, for example, Sphacelotheca reiliana; Tilletia species such as, for example, Tilletia caries; Urocystis species such as, for example, Urocystis occulta; Ustilago species such as, for example, Ustilago nuda;

fruit rots caused by, for example, Aspergillus species such as, for example, Aspergillus flavus; Botrytis species such as, for example, Botrytis cinerea; Penicillium species such as, for example, Penicillium expansum and Penicillium purpurogenum; Sclerotinia species such as, for example, Sclerotinia sclerotiorum; Verticilium species such as, for example, Verticilium alboatrum;

seed- and soil-borne rot and wilts, and seedling diseases, caused by, for example, Fusarium species such as, for example, Fusarium culmorum; Phytophthora species, such as, for example, Phytophthora cactorum; Pythium species such as, for example, Pythium ultimum; Rhizoctonia species such as, for example, Rhizoctonia solani; Sclerotium species such as, for example, Sclerotium rolfsii;

cancers, galls and witches' broom disease, caused by, for example, Nectria species such as, for example, Nectria galligena;

wilts caused by, for example, Monilinia species such as, for example, Monilinia laxa;

deformations of leaves, flowers and fruits, caused by, for example, Taphrina species such as, for example, Taphrina deformans;

degenerative diseases of woody species, caused by, for example, Esca species such as, for example, Phaeomoniella chlamydospora and Phaeoacremonium aleophilum and Fomitiporia mediterranea;

diseases of flowers and seeds, caused by, for example, Botrytis species such as, for example, Botrytis cinerea;

diseases of the plant tubers, caused by, for example, Rhizoctonia species such as, for example, Rhizoctonia solani; Helminthosporium species such as, for example, Helminthosporium solani;

diseases caused by bacterial pathogens, such as, for example, Xanthomonas species such as, for example, Xanthomonas campestris pv. oryzae; Pseudomonas species such as, for example, Pseudomonas syringae pv. lachrymans; Erwinia species such as, for example, Erwinia amylovora;

The following diseases of soybeans can preferably be controlled:

Fungal diseases on leaves, stems, pots and seeds caused by, for example,

alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllostica sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola),

fungal diseases on roots and the stem base caused by, for example,

black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defences against attack by undesired microorganisms.

Plant-strengthening (resistance-inducing) substances are understood as meaning, in the present context, those substances which are capable of stimulating the defence system of plants in such a way that, when subsequently inoculated with undesired microorganisms, the treated plants display a substantial degree of resistance to these microorganisms.

In the present case, undesired microorganisms are understood as meaning phytopathogenic fungi and bacteria. Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or else by biotechnological and genetic engineering methods or by combinations of these methods, including the transgenic plants and including the plant varieties capable or not capable of being protected by Plant Breeders' rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention with the active compounds, of the plants and plant parts, is carried out directly or by acting on their environment, habitat, or store by the customary treatment methods, for example by immersion, spraying, vaporizing, fogging, broadcasting, painting on and, in the case of propagation material, in particular in the case of seeds, furthermore by coating with one or more coats.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

In the protection of materials, the compounds according to the invention can be employed for protecting industrial materials against attack and destruction by undesired microorganisms.

In the present context, industrial materials are understood as meaning non live materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example, cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discoloring and wood-destroying fungi (Basidiomycetes) and against slime organisms and algae.

Examples which may be mentioned are microorganisms of the following genera:

Alternaria, such as Alternaria tenuis, Aspergillus, such as Aspergillus niger, Chaetomium, such as Chaetomium globosum, Coniophora, such as Coniophora puetana, Lentinus, such as Lentinus tigrinus, Penicillium, such as Penicillium glaucum, Polyporus, such as Polyporus versicolor, Aureobasidium, such as Aureobasidium pullulans, Sclerophoma, such as Sclerophoma pityophila, Trichoderma, such as Trichoderma viride, Escherichia, such as Escherichia coli, Pseudomonas, such as Pseudomonas aeruginosa, Staphylococcus, such as Staphylococcus aureus.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

Depending on their particular physical and/or chemical properties, the active compounds can be converted into the customary formulations, such as solutions, emulsions, suspensions, powders, foams, pastes, granules, aerosols, microencapsulations in polymeric substances and in coating materials for seed, and also ULV cold and warm fogging formulations.

The active compounds can be used as such, in the form of their formulations or in the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural substances impregnated with active compound, synthetic substances impregnated with active compound, fertilizers and also microencapsulations in polymeric substances. Application is carried out in a customary manner, for example by watering, spraying, atomizing, broadcasting, dusting, foaming, spreading, etc. It is furthermore possible to apply the active compounds by the ultra-low volume method or to inject the preparation of active compound or the active compound itself into the soil. It is also possible to treat the seed of the plants.

These formulations are produced in a known manner, for example by mixing the active compounds with extenders, that is, liquid solvents, pressurized liquefied gases and/or solid carriers, optionally with the use of surface-active agents, that is emulsifers and/or dispersants, and/or foam formers. If the extender used is water, it is also possible to employ for example organic solvents as cosolvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are those liquids which are gaseous at ambient temperature and at atmospheric pressure, for example aerosol propellants such as halogenated hydrocarbons and also butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as finely divided silica, alumina and silicates. As solid carriers for granules there are suitable: for example crushed and fractionated natural rocks such as calcite, pumice, marble, sepiolite and dolomite, and also synthetic granules of inorganic and organic meals, and granules of organic material such as sawdust, coconut shells, maize cobs and tobacco stalks. As emulsifiers and/or foam formers there are suitable: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates. As dispersants there are suitable: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other suitable additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.1 and 95% and particularly preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance. Examples of co-components in mixtures are the following compounds:

Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;

2) mitosis and cell division inhibitors: for example benomyl, carbendazim, diethofencarb, ethaboxam, fuberidazole, pencycuron, thiabendazole, thiophanate-methyl, zoxamide;

3) respiration inhibitors (inhibitors of the respiratory chain):

3.1) inhibitors which act on complex I of the respiratory chain: for example diflumetorim;

3.2) inhibitors which act on complex II of the respiratory chain: for example boscalid, carboxin, fenfuram, flutolanil, furametpyr, furmecyclox, mepronil, oxycarboxin, penthiopyrad, thifluzamide;

3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;

4) decouplers: for example dinocap, fluazinam, meptyldinocap;

5) ATP production inhibitors: for example fentin acetate, fentin chloride, fentin hydroxide, silthiofam;

6) amino acid and protein biosynthesis inhibitors: for example andoprim, blasticidin-S, cyprodinil, kasugamycin, kasugamycin hydrochloride hydrate, mepanipyrim, pyrimethanil;

7) signal transduction inhibitors: for example fenpiclonil, fludioxonil, quinoxyfen;

8) lipid and membrane synthesis inhibitors: for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl, vinclozolin;

9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulphate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;

10) cell wall synthesis inhibitors: for example benthiavalicarb, dimethomorph, flumorph, iprovalicarb, mandipropamid polyoxins, polyoxorim, validamycin A;

11) melanin biosynthesis inhibitors: for example carpropamid, diclocymet, fenoxanil, phthalide, pyroquilon, tricyclazole;

12) resistance inductors: for example acibenzolar-S-methyl, probenazole, tiadinil;

13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;

14) a compound selected from the following enumeration: (2E)-2-(2-{[6-(3-chloro-2-methylphenoxy)-5-fluoropyrimidin-4-yl]oxy}phenyl)-2-(methoxyimino)-N-methylacetamide, (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylat, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts, 3,4,5-trichloropyridine-2,6-dicarbonitrile, 3-[5-(4-chlorophenyl)-2,3-dimethylisoxazolidin-3-yl]pyridine, 5-chloro-6-(2,4,6-trifluorophenyl)-N-[(1R)-1,2,2-trimethylpropyl][1,2,4]triazolo[1,5-a]pyrimidine-7-amine, 5-chloro-7-(4-methylpiperidin-1-yl)-6-(2,4,6-trifluorophenyl)[1,2,4]triazolo[1,5-a]pyrimidine, 5-chloro-N-[(1R)-1,2-dimethylpropyl]-6-(2,4,6-trifluorophenyl)[1,2,4]triazolo[1,5-a]pyrimidine-7-amine, 8-hydroxyquinoline sulphate, benthiazole, bethoxazin, capsimycin, carvone, chinomethionat, cufraneb, cyflufenamid, cymoxanil, dazomet, debacarb, dichlorophen, diclomezine, dicloran, difenzoquat, difenzoquat methylsulphate, diphenylamine, ferimzone, flumetover, fluopicolide, fluoroimide, flusulfamide, fosetyl-aluminium, fosetyl-calcium, fosetyl-sodium, hexachlorobenzene, irumamycin, isotianil, methasulfocarb, methyl(2E)-2-{2-[{cyclopropyl[(4-methoxyphenyl)-imino]methyl}thio)methyl]phenyl}-3-methoxyacrylate, methyl 1-(2,2-dimethyl-2,3-dihydro-1H-inden-1)-1H-imidazol-5-carboxylate, methyl isothiocyanate, metrafenone, mildiomycin, N-[2-(1,3-dimethylbutyl)phenyl]-5-fluoro-1,3-dimethyl-1H-pyrazole-4-carboxamide, N-(3′,4′-dichloro-5-fluorobiphenyl-2-yl)-3-(difluoromethyl)-1-methyl-1H-pyrazole-4-carboxamide(bixafen), N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-(4-chlorobenzyl)-3-[3-methoxy-4-(prop-2-yn-1-yloxy)phenyl]-propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N-(methylsulphonyl)valinamide, N-{(Z)-[(cyclopropylmethoxy)imino][6-(difluoromethoxy)-2,3-difluorophenyl]methyl}-2-phenylacetamide, N-{2-[1,1′-bi(cyclopropyl)-2-yl]phenyl}-3-(difluoromethyl)-1-methyl-1H-pyrazole-4-carboxamide, N-{2[3-chloro-5-(trifluoromethyl)pyridin-2-yl]ethyl}-2-(trifluoromethyl)benzamide(fluopyram), natamycin, N-ethyl-N-methyl-N′-{2-methyl-5-(trifluoromethyl)-4-[3-(trimethylsilyl)propoxy]phenyl}imidoformamide, N-ethyl-N-methyl-N′-{2-methyl-5-(difluoromethyl)-4-[3-(trimethylsilyl)propoxy]phenyl}imidoformamide, Nickel dimethyldithiocarbamate, nitrothal-isopropyl, O-{1-[(4-methoxyphenoxy)methyl]-2,2-dimethylpropyl{1H-imidazole-1-carbothioate, octhilinone, oxamocarb, oxyfenthiin, pentachlorophenol and salts, phosphoric acid and salts thereof, piperalin, propamocarb-fosetylat, propanosine-sodium, proquinazid, pyribencarb, pyrrolnitrins, quintozene, S-allyl 5-amino-2-isopropyl-4-(2-methylphenyl)-3-oxo-2,3-dihydro-1H-pyrazole-1-carbothioate, tecloftalam, tecnazene, triazoxide, trichlamide, valiphenal, zarilamid.

The active compounds according to the invention can also be mixed with known bactericides and insecticides/acaricides/nematicides.

A mixture with other known active compounds such as herbicides, or with fertilizers and growth regulators, safeners or semiochemicals is also possible.

In addition, the compounds of the formulae (Ia), (Ib), (Ic), (Id),(Ie) and (If) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The active compounds can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, broadcasting, dusting, foaming, painting on and the like. It is furthermore possible to apply the active compounds by the ultra-low-volume method, or to inject the active compound preparation or the active compound itself into the soil. The seed of the plant can also be treated.

When employing the active compounds according to the invention as fungicides, the application rates can be varied within a substantial range, depending on the type of application. In the treatment of plant parts, the application rates of active compound are generally between 0.1 and 10 000 g/ha, preferably between 10 and 1000 g/ha. For the treatment of seed, the application rates of active compound are generally between 0.001 and 50 g per kilogram of seed, preferably between 0.01 and 10 g per kilogram of seed. For treating the soil, the application rates of active compound are generally between 0.1 and 10 000 g/ha, preferably between 1 and 5000 g/ha.

The application rates are mentioned as examples only and are not limiting in the sense of the invention.

The active compound or compositions according to the invention can also be used for protecting plants for a certain period of time after treatment against attack by the pathogens mentioned. The period of time for which protection is provided generally extends over 1 to 28 days, preferably 1 to 14 days, after treatment of the plants with the active compounds or for up to 200 days after seed treatment.

The control of phytopathogenic fungi by treating the seeds of plants has been known for a long time and is subject-matter of continuous improvements. However, the treatment of seed frequently entails a series of problems which cannot always be solved in a satisfactory manner. Thus, it is desirable to develop methods for protecting the seed and the germinating plant which dispense with the additional application of crop protection agents after sowing or after the emergence of the plants or where additional applications are at least reduced. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular also relates to a method for the protection of seed and germinating plants from attack by phytopathogenic fungi, by treating the seed with a composition according to the invention.

The invention likewise relates to the use of the compositions according to the invention for the treatment of seed for protecting the seed and the germinating plant from phytopathogenic fungi.

Furthermore, the invention relates to seed which has been treated with a composition according to the invention so as to afford protection from phytopathogenic fungi.

One of the advantages of the present invention is that the particular systemic properties of the compositions according to the invention mean that treatment of the seed with these compositions not only protects the seed itself, but also the resulting plants after emergence, from phytopathogenic fungi. In this manner, the immediate treatment of the crop at the time of sowing or shortly thereafter can be dispensed with.

Furthermore, it must be considered as advantageous that the mixtures according to the invention can also be employed in particular in transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants. The treatment of seed of cereals (such as wheat, barley, rye and oats), maize and rice is of particular importance.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, for example, it is possible to use seed which has been harvested, cleaned and dried to a moisture content of below 15% by weight. Alternatively, it is also possible to use seed which, after drying, has, for example, been treated with water and then dried again.

When treating the seed, care must generally be taken that the amount of the composition according to the invention applied to the seed and/or the amount of further additives is chosen in such a way that the germination of the seed is not adversely affected, or that the resulting plant is not damaged.

This must be borne in mind in particular in the case of active compounds which may have phytotoxic effects at certain application rates.

The compositions according to the invention can be applied directly, that is to say without comprising further components and without having been diluted. In general, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations which can be used according to the invention can be converted into customary seed dressing formulations, such as solutions, emulsions, suspensions, powders, foams, slurries or other coating materials for seed, and also ULV formulations.

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and water as well.

Suitable colorants that may be present in the seed dressing formulations which can be used according to the invention include all colorants customary for such purposes. Use may be made both of pigments, of sparing solubility in water, and of dyes, which are soluble in water. Examples that may be mentioned include the colorants known under the designations rhodamine B, C.I. Pigment Red 112, and C.I. Solvent Red 1.

Suitable wetting agents that may be present in the seed dressing formulations which can be used according to the invention include all substances which promote wetting and are customary in the formulation of active agrochemical substances. With preference it is possible to use alkylnaphthalene-sulphonates, such as diisopropyl- or diisobutylnaphthalene-sulphonates.

Suitable dispersants and/or emulsifiers that may be present in the seed dressing formulations which can be used according to the invention include all nonionic, anionic, and cationic dispersants which are customary in the formulation of active agrochemical substances. With preference, it is possible to use nonionic or anionic dispersants or mixtures of nonionic or anionic dispersants. Particularly suitable nonionic dispersants are ethylene oxide-propylene oxide block polymers, alkylphenol polyglycol ethers, and tristyrylphenol polyglycol ethers, and their phosphated or sulphated derivatives. Particularly suitable anionic dispersants are lignosulphonates, polyacrylic salts, and arylsulphonate-formaldehyde condensates.

Suitable defoamers that may be present in the seed dressing formulations which can be used according to the invention include all foam-inhibiting substances which are customary in the formulation of active agrochemical substances. With preference it is possible to use silicone defoamers and magnesium stearate.

Suitable preservatives that may be present in the seed dressing formulations which can be used according to the invention include all substances which can be used for such purposes in agrochemical compositions. By way of example, mention may be made of dichlorophen and benzyl alcohol hemiformal.

Suitable secondary thickeners that may be present in the seed dressing formulations which can be used according to the invention include all substances which can be used for such purposes in agrochemical compositions. Preferred suitability is possessed by cellulose derivatives, acrylic acid derivatives, xanthan, modified clays, and highly disperse silica.

Suitable adhesives that may be present in the seed dressing formulations which can be used according to the invention include all customary binders which can be used in seed dressing. With preference, mention may be made of polyvinylpyrrolidone, polyvinyl acetate, polyvinyl alcohol and tylose.

Suitable gibberellins that may be present in the seed dressing formulations which can be used according to the invention are preferably the gibberellins A1, A3 (=gibberellinic acid), A4 and A7; particularly preferably, gibberellinic acid is used. The gibberellins are known (cf. R. Wegler, Chemie der Pflanzenschutz- and Schädlingsbekämpfungsmittel“, Vol. 2, Springer Verlag, 1970, pp. 401-412).

The seed dressing formulations which can be used according to the invention may be used directly or after dilution with water beforehand to treat seed of any of a very wide variety of types. For instance, the concentrates or the preparations obtainable therefrom by dilution with water may be used to dress the seed of cereals, such as wheat, barley, rye, oats, and triticale, and also the seed of maize, rice, oilseed rape, peas, field beans, cotton, sunflowers, and beets, or else vegetable seed of any of a very wide variety of kinds. The seed dressing formulations which can be used according to the invention or their dilute preparations may also be used to dress seed of transgenic plants. In this context, synergistic effects may also arise in interaction with the substances formed by expression.

Suitable mixing equipment for treating seed with the seed dressing formulations which can be used according to the invention or the preparations prepared from them by adding water includes all mixing equipment which can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

The application rate of the seed dressing formulations which can be used according to the invention may be varied within a relatively wide range. It depends on the respective content of the active compounds in the formulations and on the seed. In general, the application rates of active compound combination are between 0.001 and 50 g per kilogram of seed, preferably between 0.01 and 15 g per kilogram of seed.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, further developed root system, higher resistance of the plant species or plant cultivars, increased growth of the shoots, higher plant vitality, increased tolerance to high or low temperatures, increased tolerance to drought or to water or soil salt content, increased flowering performance, easier harvesting, accelerated maturation, higher harvest yields, larger fruits, larger plant sizes, greener color of the leaves, earlier flowering, higher quality and/or a higher nutritional value of the harvested products, higher sugar concentration in the fruits, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particular advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

Process 1 (cf. Scheme 1)

Step 1:

### 2,5-Dichloro-N-cyclopropylpyrimidine-4-amine

At −10° C., 56.5 g (409 mmol) of potassium carbonate is added to a solution of 50.0 g (272 mmol) 2,4,5-trichloropyrimidine in 480 ml of acetonitrile. Subsequently, 16.2 g (286 mmol) of cyclopropylamine as a 30% strength solution in acetonitrile are then added dropwise over a period of 30 min. With stirring, the reaction mixture is allowed to warm to room temperature overnight. Under reduced pressure, the reaction mixture is freed from the solvent. 1000 ml of ice-water/dilute hydrochloric acid (1:1) are added to the residue. The precipitated solid is filtered off, washed with water (2×250 ml) and dried in the air. The crude product is stirred into 100 ml of petroleum ether, and the mixture is stirred at room temperature for another 1 h, filtered again and dried until the weight remains constant. This gives 55.0 g (97%) of the desired product logP (pH2.3): 1.79.

The following compounds can be prepared in an analogous manner:
5-bromo-2-chloro-N-cyclopropylpyrimidine-4-amine logP (pH2.3): 1.97
2-chloro-N-cyclopropyl-5-fluoropyrimidine-4-amine logP (pH2.3): 1.42
(CAS: 893772-23-1 commercially available from Aurora Screening Library, Graz, Austria)
2-chloro-N-cyclopropyl-5-methoxypyrimidine-4-amine logP (pH2.3): 1.38
2-chloro-N-cyclopropyl-5-methylpyrimidine-4-amine logP (pH2.3): 1.28
2-chloro-N-cyclopropyl-5-iodopyrimidine-4-amine logP (pH2.3): 2.19
2-chloro-5-cyano-N-cyclopropylpyrimidine-4-amine described in WO 2002004429
2,5-dichloro-N-(cyclopropylmethyl)pyrimidine-4-amine logP (pH2.3): 2.51
2,5-dichloro-N-(1-cyclopropylethyl)pyrimidine-4-amine logP (pH2.3): 2.97
5-bromo-2-chloro-N-(cyclopropylmethyl)pyrimidine-4-amine logP (pH2.3): 2.69
(WO2003076437, WO2002096888)
2-chloro-N-cyclopropyl-5-trifluoromethylpyrimidine-4-amine

At room temperature, 3.8 ml (27.6 mmol) of triethylamine and then 0.79 g (13.8 mmol) of cyclopropylamin are added to a solution of 3.00 g (13.8 mmol) of 2,4-dichloro-5-trifluoropyrimidine in 30 ml of dioxane. The mixture is stirred at 95° C. for 16 h. After cooling, the reaction mixture is stirred into ice-water and extracted with ethyl acetate (3×150 ml). The combined organic phases are separated off, washed with water, dried over MgSO₄ and freed from the solvent under reduced pressure. The crude product is purified by column chromatography on RP-C18 (water/acetonitrile). This gives 520 mg (16%) of the desired product. logP (pH2.3): 2.39

The following compound can be prepared analogously:
2-chloro-N-(cyclopropylmethyl)-5-trifluoromethylpyrimidine-4-amine logP (pH2.3): 3.40
2,5-Dichloro-N-(1-ethoxycyclopropyl)pyrimidine-4-amine

At −15° C., 5.65 g (40.9 mmol) of potassium carbonate are added to a solution of 3.00 g (16.3 mmol) of 2,4,5-trichloropyrimidine in 30 ml of acetonitrile. A little at a time, 2.36 g (17.2 mmol) of 1-ethoxycyclopropanaminr hydrochloride (Helvetica Chimica Acta, 1992, 75, 1078-84) are then added. The mixture is stirred at room temperature for 16 h and at 40° C. for 5 h. The reaction mixture is stirred into ixw-water and extracted with dichloromethane (3×150 ml). The combined organic phases are separated off, washed with water, dried over MgSO₄ and freed from the solvent under reduced pressure. The crude product is stirred into 50 ml cyclohexane, and the mixture is stirred at room temperature for another 1 h. The precipitated solid is filtered off and dried until the weight remains constant. This gives 750 mg (18%) of the desired product. logP (pH2.3): 2.09.

Step 2 (cf. Scheme 2)

Method A:

### 5-Chloro-N⁴-(cyclopropylmethyl)-N²-(4-isopropoxyphenyl)pyrimidine-2,4-diamine hydrochloride (Ex. 55)

At room temperature, 400 μl of a 4 M solution of HCl in dioxane are added to a solution of 218 mg (1.00 mmol) of 2,5-dichloro-N-(cyclopropylmethyl)pyrimidine-4-amine and 299 mg (1.98 mmol) of 4-(isopropoxy)aniline in 12 ml of acetonitrile, and the mixture is heated at 85° C. After 16 h, the hot reaction mixture is filtered and the filtrate is allowed to cool with stirring. The product precipitated from the filtrate is filtered off and dried. This gives 130 mg (35%). logP (pH2.3): 2.11

Method B:

### 3-{[5-Chloro-4-(cyclopropylamino)pyrimidin-2-yl]amino}benzenesulphonamide (Ex. 15)

A mixture of 250 mg (1.22 mmol) of 2,5-dichloro-N-cyclopropylpyrimidine-4-amine, 264 mg (1.53 mmol) of 3-aminobenzenesulphonamide and 169 mg (0.98 mmol) of 4-toluenesulphonic acid in 12 ml of dioxane is stirred at 105° C. for 16 h. After cooling, the precipitated solid is filtered off, suspended in 10 ml of water, washed with water (2×10 ml) and dried. This gives 269 mg (65%) of the desired product. logP (pH2.3): 1.37

### N⁴-Cyclopropyl-5-iodo-N²-phenylpyrimidine-2,4-diamine

A mixture of 250 mg (0.85 mmol) of 2-chloro-N-cyclopropyl-5-iodopyrimidine-4-amine, 98 mg (1.06 mmol) of aniline and 58 mg (0.34 mmol) of 4-toluenesulphonic acid in 12 ml of dioxane is stirred at 60° C. for 7 h. After cooling, the resulting precipitate is filtered off, washed with 5 ml of dioxane, suspended in 10 ml of water, neutralized with sat. NaHCO₃ solution, washed with water (2×5 ml) and dried. This gives 106 mg (36%) of the desired product. logP (pH2.3): 1.82.

### 4-{[5-Chloro-4-(cyclopropylamino)pyrimidin-2-yl]amino}-N-isopropylbenzamide (Ex. 43)

A mixture of 5.57 g (15.7 mmol) of methyl 4-{[5-chloro-4-(cyclopropylamino)pyrimidin-2-yl]amino}benzoate hydrochloride (prepared according to Route A, Method A) and 50 ml of a 1 M NaOH solution in 100 ml of MeOH is stirred at 50° C. for 20 h, until a solution is formed. After cooling, the reaction solution is acidified with conc. HCl to pH 2. The precipitated solid is filtered off, washed with 50 ml of water and dried. This gives 4.6 g (96%) of 4-{[5-chloro-4-(cyclopropylamino)pyrimidin-2-yl]amino}benzoic acid. logP (pH2.3): 1.55

1 drop of DMF and 360 mg (3.82 mmol) of thionyl chloride are added dropwise to 500 mg (1.64 mmol) of 4-{[5-chloro-4-(cyclopropylamino)pyrimidin-2-yl]amino}benzoic acid in 20 ml of 1,2-dichloroethane. After 16 h of stirring at 50° C., the reaction mixture is concentrated under reduced pressure. This gives 520 mg (98%) of 4-{[5-chloro-4-(cyclopropylamino)pyrimidin-2-yl]amino}benzoyl chloride, which is directly reacted further.

A mixture of 200 mg (0.62 mmol) of 4-{[5-chloro-4-(cyclopropylamino)pyrimidin-2-yl]amino}benzoyl chloride, 80 mg (1.36 mmol) of isopropylamine and 128 mg (0.93 mmol) of potassium carbonate in 5 ml of acetonitrile is stirred for 2 h. The reaction mixture is then stirred into 20 ml of ice-water, and the resulting precipitate is filtered off and dried. This gives 150 mg (70%) of 4-{[5-chloro-4-(cyclopropylamino)pyrimidin-2-yl]amino}-N-isopropylbenzamide. logP (pH2.3): 1.73

Process 2b (cf. Scheme 3)

Step 1:

### 2-Anilino-5-chloropyrimidin-4(3H)-one

A solution consisting of 3.27 ml of a 1 M NaOH (aq) and 1 ml of water is added to a solution of 500 mg (2.73 mmol) of 2,4,5-trichloropyrimidine in 10 ml of dioxane. After 4 d of stirring at room temperature, the reaction mixture is concentrated under reduced pressure. The residue is taken up in 50 ml of ethyl acetate and neutralized with 1 N HCl (aq). The organic phase is separated off and then washed with 10 ml of water, dried over MgSO₄ and freed from the solvent under reduced pressure. The crude product is, together with 424 mg (4.55 mmol) of aniline and 532 mg (3.09 mmol) of 4-toluenesulphonic acid, taken up in 10 ml of dioxane and heated at 105° C. with stirring. After 18 h, the reaction mixture is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 1000 mg of crude product, which is directly reacted further without further purification. logP (pH2.3): 1.56.

Step 2:

### 4,5-Dichloro-N-phenylpyrimidine-2-amine

A solution of 400 mg of 2-anilino-5-chloropyrimidin-4(3H)-one in 2 ml of phophoryl chloride is heated at 95° C. for 18 h. After cooling, the reaction mixture is concentrated under reduced pressure, added to water and extracted with dichloromethane (3×20 ml). The combined organic phases are dried over MgSO₄ and freed from the solvent under reduced pressure. This gives 450 mg. logP (pH2.3): 3.52

Step 3:

### 5-Chloro-N⁴-cyclopropyl-N²-phenylpyrimidine-2,4-diamine (Ex. 2) (cf. Scheme 5)

At 0° C., 100 mg (0.99 mmol) of triethylamine and 66.9 mg (1.17 mmol) of cyclopropylamine are added to a solution of 216 mg (0.90 mmol) of 4,5-dichloro-N-phenylpyrimidine-2-amine in 15 ml of acetonitrile. After 18 h of stirring at room temperature, the reaction mixture is added to water and extracted with ethyl acetate (5×40 ml). The combined organic phases are dried over MgSO₄ and freed from the solvent under reduced pressure. This gives 280 mg of the desired product in a purity of 60%. (logP (pH2.3): 1.61

### EXAMPLES

The compounds of the formulae (I), (Ia), (Ib), (Ic), (Id), (Ie) and (If) listed in Table 1 below are or were obtained analogously to the method given above.

| | |
|---|---|
| 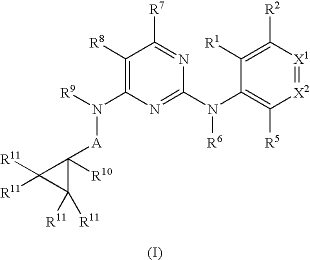 | 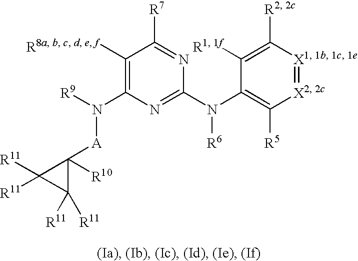 |

| Ex. | X¹, X^{1b} X^{1c}, X^{1e} | X², X^{2c} | R¹ R^{1f} | R² R^{2c} | R³ or —R² + R³— | R⁴ | R⁵ | R⁶ | R⁷ | R⁸ R^{8a} R^{8b} R^{8c} R^{8d} R^{8e} R^{8f} | R⁹ | A | Coun- ter ion | 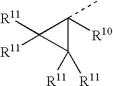 | logP^{a} |
|---|---|---|---|---|---|---|---|---|---|---|---|---|---|---|---|
| 1 | CR³ | CR⁴ | H | H | H | Cl | H | H | H | Cl | H | bond | — | cyclopropyl | 2.7* |
| 2 | CR³ | CR⁴ | H | H | H | H | H | H | H | Cl | H | bond | — | cyclopropyl | 1.61* |
| 3 | CR³ | CR⁴ | H | OMe | H | OMe | H | H | H | Cl | H | bond | — | cyclopropyl | 1.91* |
| 4 | CR³ | CR⁴ | H | OMe | OMe | OMe | H | H | H | Cl | H | bond | — | cyclopropyl | 1.73* |
| 5 | CR³ | CR⁴ | H | H | H | COMe | H | H | H | Cl | H | bond | — | cyclopropyl | 1.84** |
| 6 | CR³ | CR⁴ | H | H | SO₂NMe₂ | H | H | H | H | Cl | H | bond | — | cyclopropyl | 2.26** |
| 7 | CR³ | CR⁴ | H | H | 4-(tert-butoxy- | H | H | H | H | Cl | H | bond | — | cyclopropyl | 2.4* |
| | | | | | carbonyl)- | | | | | | | | | | |
| | | | | | piperazin-1-yl | | | | | | | | | | |
| 8 | CR³ | CR⁴ | H | H | SO₂NH₂ | H | H | H | H | Cl | H | methylene | — | cyclopropyl | 1.52** |
| 9 | CR³ | CR⁴ | H | H | H | Cl | H | H | H | Cl | H | methylene | — | cyclopropyl | 2.7** |
| 10 | CR³ | CR⁴ | H | H | H | H | H | H | H | Cl | H | methylene | — | cyclopropyl | 1.85** |
| 11 | CR³ | CR⁴ | H | OMe | H | OMe | H | H | H | Cl | H | methylene | — | cyclopropyl | 2.09** |
| 12 | CR³ | CR⁴ | H | OMe | OMe | OMe | H | H | H | Cl | H | methylene | — | cyclopropyl | 1.78** |
| 13 | CR³ | CR⁴ | H | H | H | COMe | H | H | H | Cl | H | methylene | — | cyclopropyl | 1.87** |
| 14 | CR³ | CR⁴ | H | H | SO₂NMe₂ | H | H | H | H | Cl | H | methylene | — | cyclopropyl | 2.36** |
| 15 | CR³ | CR⁴ | H | H | H | SO₂NH₂ | H | H | H | Cl | H | bond | — | cyclopropyl | 1.37** |
| 16 | CR³ | CR⁴ | H | H | H | SO₂NH₂ | H | H | H | Cl | H | methylene | — | cyclopropyl | 1.51** |
| 17 | CR³ | CR⁴ | H | CF₃ | H | SO₂NH₂ | H | H | H | Cl | H | bond | — | cyclopropyl | 2.48** |
| 18 | CR³ | CR⁴ | H | CF₃ | H | SO₂NH₂ | H | H | H | Cl | H | methylene | — | cyclopropyl | 2.63** |
| 19 | CR³ | CR⁴ | H | H | H | morpholin-4- | H | H | H | Cl | H | bond | — | cyclopropyl | 2.13** |
| | | | | | | ylsulphonyl | | | | | | | | | |
| 20 | CR³ | CR⁴ | H | H | (methylamino)- | H | H | H | H | Cl | H | methylene | — | cyclopropyl | 1.87** |
| | | | | | sulphonyl | | | | | | | | | | |
| 21 | CR³ | CR⁴ | H | H | (methylamino)- | H | H | H | H | Cl | H | bond | — | cyclopropyl | 1.71** |
| | | | | | sulphonyl | | | | | | | | | | |
| 22 | CR³ | CR⁴ | H | H | (butylamino)- | H | H | H | H | Cl | H | bond | — | cyclopropyl | 2.6** |
| | | | | | sulphonyl | | | | | | | | | | |
| 23 | CR³ | CR⁴ | H | H | (butylamino)- | H | H | H | H | Cl | H | methylene | — | cyclopropyl | 2.75** |
| | | | | | sulphonyl | | | | | | | | | | |
| 24 | CR³ | CR⁴ | H | H | acetamido- | H | H | H | H | Cl | H | bond | — | cyclopropyl | 1.64** |
| | | | | | sulphonyl | | | | | | | | | | |
| 25 | CR³ | CR⁴ | H | H | SO₂NH₂ | H | H | H | H | Cl | H | bond | — | cyclopropyl | 1.37** |
| 26 | CR³ | CR⁴ | H | H | [(4,6- | H | H | H | H | Cl | H | bond | — | cyclopropyl | 1.72** |
| | | | | | dimethylpyrimi- | | | | | | | | | | |
| | | | | | din-2-yl)- | | | | | | | | | | |
| | | | | | amino]sulphonyl | | | | | | | | | | |
| 27 | CR³ | CR⁴ | H | H | anilino- | H | H | H | H | Cl | H | bond | — | cyclopropyl | 2.56** |
| | | | | | sulphonyl | | | | | | | | | | |
| 28 | CR³ | CR⁴ | H | H | H | SO₂NMe₂ | H | H | H | Cl | H | bond | — | cyclopropyl | 2.04** |
| 29 | CR³ | CR⁴ | H | H | H | H | H | H | H | Cl | H | bond | HCl | cyclopropyl | 1.75** |
| 30 | CR³ | CR⁴ | H | H | H | H | H | H | H | Br | H | bond | — | cyclopropyl | 1.8** |
| 31 | CR³ | CR⁴ | H | H | (allylamino) | H | H | H | H | Cl | H | bond | — | cyclopropyl | 2.09** |
| | | | | | sulphonyl | | | | | | | | | | |
| 32 | CR³ | CR⁴ | H | H | SO₂NH₂ | H | H | H | H | Br | H | bond | — | cyclopropyl | 1.48** |
| 33 | CR³ | CR⁴ | H | H | H | 2-oxopyrrol- | H | H | H | Cl | H | bond | — | cyclopropyl | 1.52** |
| | | | | | | idin-1-yl | | | | | | | | | |
| 34 | CR³ | CR⁴ | H | H | 2- | H | H | H | H | Cl | H | bond | — | cyclopropyl | 1.45** |
| | | | | | oxopyrrolidin- | | | | | | | | | | |
| | | | | | 1-yl | | | | | | | | | | |
| 35 | CR³ | CR⁴ | H | H | COOH | H | H | H | H | Cl | H | bond | — | cyclopropyl | 1.55** |
| 36 | CR³ | CR⁴ | H | H | methoxycarbonyl | H | H | H | H | Cl | H | bond | HCl | cyclopropyl | 2.25** |
| 37 | CR³ | CR⁴ | H | H | methoxycarbonyl | H | H | H | H | Cl | H | bond | — | cyclopropyl | 2.23* |
| 38 | CR³ | CR⁴ | H | H | H | H | H | H | H | Cl | H | bond | HCl | 1-ethoxy- | 2.23** |
| | | | | | | | | | | | | | | cyclopropyl | |
| 39 | CR³ | CR⁴ | H | H | H | H | H | H | H | Cl | H | bond | — | 1-ethoxy- | 2.23** |
| | | | | | | | | | | | | | | cyclopropyl | |
| 40 | CR³ | CR⁴ | H | H | H | H | H | H | H | CN | H | bond | — | cyclopropyl | 2.44* |
| 41 | CR³ | CR⁴ | H | H | H | H | H | H | H | F | H | bond | HCl | cyclopropyl | 1.45** |
| 42 | CR³ | CR⁴ | H | H | H | H | H | H | H | F | H | bond | — | cyclopropyl | 1.45** |
| 43 | CR³ | CR⁴ | H | H | isopropyl- | H | H | H | H | Cl | H | bond | — | cyclopropyl | 1.73** |
| | | | | | carbamoyl | | | | | | | | | | |
| 44 | CR³ | CR⁴ | H | H | COMe | H | H | H | H | Cl | H | bond | — | cyclopropyl | 1.99* |
| 45 | CR³ | CR⁴ | H | H | COMe | H | H | H | H | Cl | H | bond | HCl | cyclopropyl | 1.99** |
| 46 | CR³ | CR⁴ | H | H | SO₂NH₂ | H | H | H | H | F | H | bond | — | cyclopropyl | 0.95** |
| 47 | CR³ | CR⁴ | H | H | Me | SO₂NMe₂ | H | H | H | Cl | H | bond | — | cyclopropyl | 2.14** |
| 48 | CR³ | CR⁴ | H | H | [(methylamino) | H | H | H | H | Cl | H | bond | — | cyclopropyl | 1.37** |
| | | | | | sulphonyl]methyl | | | | | | | | | | |
| 49 | CR³ | CR⁴ | H | H | H | SO₂NMe₂ | H | H | H | Cl | H | methylene | — | cyclopropyl | 2.18** |
| 50 | CR³ | CR⁴ | H | H | methylsulphonyl | H | H | H | H | Cl | H | bond | — | cyclopropyl | 1.75** |
| 51 | CR³ | CR⁴ | H | H | H | H | H | H | H | Me | H | bond | — | cyclopropyl | 1.5* |
| 52 | CR³ | CR⁴ | H | OMe | OMe | OMe | H | H | H | Me | H | bond | — | cyclopropyl | 1.47* |
| 53 | CR³ | CR⁴ | H | H | 1H-tetrazol-5-yl | H | H | H | H | Cl | H | bond | — | cyclopropyl | 1.47* |
| 54 | CR³ | CR⁴ | H | H | isopropyl | H | H | H | H | Cl | H | methylene | — | cyclopropyl | 2.62** |
| 55 | CR³ | CR⁴ | H | H | isopropoxy | H | H | H | H | Cl | H | methylene | HCl | cyclopropyl | 2.11** |
| 56 | CR³ | CR⁴ | H | H | —NHCOCH₂— | | H | H | H | Cl | H | bond | — | cyclopropyl | 1.05* |
| 57 | CR³ | CR⁴ | H | H | H | Cl | H | H | H | Me | H | bond | — | cyclopropyl | 1.8* |
| 58 | CR³ | CR⁴ | H | H | acetyl(methyl)- | H | H | H | H | Cl | H | bond | — | cyclopropyl | 1.4* |
| | | | | | amino | | | | | | | | | | |
| 59 | CR³ | CR⁴ | H | H | 3,3-dimethyl-2- | H | H | H | H | Cl | H | bond | — | cyclopropyl | 2.3* |
| | | | | | oxobutyl | | | | | | | | | | |
| 60 | CR³ | CR⁴ | H | H | (methoxycarbo- | H | H | H | H | Cl | H | bond | — | cyclopropyl | 1.42* |
| | | | | | nyl)amino | | | | | | | | | | |
| 61 | CR³ | CR⁴ | H | H | allylsulphonyl | H | H | H | H | Cl | H | bond | — | cyclopropyl | 2.18* |
| 62 | CR³ | CR⁴ | H | H | (cyanomethyl)- | H | H | H | H | Cl | H | bond | — | cyclopropyl | 2.2* |
| | | | | | sulphonyl | | | | | | | | | | |
| 63 | CR³ | CR⁴ | H | H | acetyl(cyclopro- | H | H | H | H | Cl | H | bond | — | cyclopropyl | 1.62* |
| | | | | | pyl)-amino | | | | | | | | | | |
| 64 | CR³ | CR⁴ | H | H | [(1- | H | H | H | H | Cl | H | bond | — | cyclopropyl | 1.66* |
| | | | | | methylcyclopro- | | | | | | | | | | |
| | | | | | pyl)carbonyl]- | | | | | | | | | | |
| | | | | | amino | | | | | | | | | | |
| 65 | CR³ | CR⁴ | H | H | —CH═CH—NH— | | H | H | H | Cl | H | bond | — | cyclopropyl | 1.57* |
| 66 | CR³ | CR⁴ | H | H | benzoylamino | H | Me | H | H | Cl | H | bond | — | cyclopropyl | 1.84* |
| 67 | CR³ | CR⁴ | H | H | (3-fluoro-2,2- | H | H | H | H | Cl | H | bond | — | cyclopropyl | 1.74* |
| | | | | | dimethylpropan- | | | | | | | | | | |
| | | | | | oyl)-amino | | | | | | | | | | |
| 68 | CR³ | CR⁴ | H | H | —N═C(CF₃)—NH— | | H | H | H | Cl | H | bond | — | cyclopropyl | 1.55* |
| 69 | CR³ | CR⁴ | H | H | SMe | H | H | H | H | Cl | H | bond | — | cyclopropyl | 2.1* |
| 70 | CR³ | CR⁴ | H | H | H | H | H | H | H | Cl | H | bond | — | cyclopropyl | 1.28* |
| 71 | CR³ | CR⁴ | H | H | —NHCH═CH— | | H | H | H | Cl | H | bond | — | cyclopropyl | 1.47* |
| 72 | CR³ | CR⁴ | H | H | NHCOtBu | H | H | H | H | Cl | H | bond | — | cyclopropyl | 1.81* |
| 73 | CR³ | CR⁴ | H | H | trifluoroacetyl | H | H | H | H | Cl | H | bond | — | cyclopropyl | 3.62* |
| 74 | CR³ | CR⁴ | H | H | H | SMe | H | H | H | Cl | H | bond | — | cyclopropyl | 2.21* |
| 75 | CR³ | CR⁴ | H | H | hydroxyl | H | H | H | H | Cl | H | bond | — | cyclopropyl | 1.12* |
| 76 | CR³ | CR⁴ | H | H | H | H | H | H | H | Cl | H | bond | — | cyclopropyl | 1.84* |
| 77 | CR³ | CR⁴ | H | H | H | hydroxy | H | H | H | Cl | H | bond | — | cyclopropyl | 1.3* |
| 78 | CR³ | CR⁴ | H | H | SO₂NMe₂ | H | H | H | H | Cl | H | bond | HCl | cyclopropyl | 2.26* |
| 79 | CR³ | CR⁴ | H | H | —N═C(CH₃)NHSO₂— | | H | H | H | Cl | H | bond | | cyclopropyl | 1.32* |
| 80 | CR³ | CR⁴ | H | H | F | H | H | H | H | Cl | H | bond | — | cyclopropyl | 1.8* |
| 81 | CR³ | CR⁴ | H | —N═C—NHCO₂— | | H | H | H | H | Cl | H | bond | | cyclopropyl | 1.5* |
| 82 | CR³ | CR⁴ | H | H | H | hydroxymethyl | H | H | H | Cl | H | bond | — | cyclopropyl | 1.22* |
| 83 | CR³ | CR⁴ | H | H | 2-oxo-1,3- | H | H | H | H | Cl | H | bond | — | cyclopropyl | 1.39* |
| | | | | | oxazolidin-3-yl | | | | | | | | | | |
| 84 | CR³ | CR⁴ | H | H | H | (cyclopropyl- | H | H | H | Cl | H | bond | — | cyclopropyl | 1.62* |
| | | | | | | carbonyl)amino | | | | | | | | | |
| 85 | CR³ | CR⁴ | H | H | morpholin-4- | H | H | H | H | Cl | H | bond | — | cyclopropyl | 2.68* |
| | | | | | yl)-sulphonyl | | | | | | | | | | |
| 86 | CR³ | CR⁴ | H | H | H | NHCOisoPr | H | H | H | Cl | H | bond | — | cyclopropyl | 1.63* |
| 87 | CR³ | CR⁴ | H | H | H | H | H | H | H | CN | H | methylene | — | cyclopropyl | 2.77* |
| 88 | CR³ | CR⁴ | H | 2- | H | H | H | H | H | Cl | H | bond | TsOH | cyclopropyl | 1.49* |
| | | | | oxopyrro- | | | | | | | | | | | |
| | | | | lidin-1-yl | | | | | | | | | | | |
| 89 | CR³ | CR⁴ | H | H | 2- | H | H | H | H | Cl | H | bond | TsOH | cyclopropyl | 1.44* |
| | | | | | oxopyrrolidin- | | | | | | | | | | |
| | | | | | 1-yl | | | | | | | | | | |
| 90 | CR³ | CR⁴ | H | H | CH₂NHCOOtBu | H | H | H | H | Cl | H | bond | — | cyclopropyl | 2.15* |
| 91 | CR³ | CR⁴ | H | H | H | NHCOtBu | H | H | H | Cl | H | bond | — | cyclopropyl | 1.98* |
| 92 | CR³ | N | H | H | OMe | — | H | H | H | Cl | H | bond | — | cyclopropyl | 1.34* |
| 93 | CR³ | CR⁴ | H | H | H | H | H | H | H | CF₃ | H | bond | — | cyclopropyl | 2.62** |
| 94 | CR³ | CR⁴ | H | H | (difluoromethyl) | H | H | H | H | Cl | H | bond | — | cyclopropyl | 2.77* |
| | | | | | thio | | | | | | | | | | |
| 95 | CR³ | CR⁴ | H | H | {[(benzyloxy) | H | H | H | H | Cl | H | bond | — | cyclopropyl | 2.2* |
| | | | | | carbonyl]amino} | | | | | | | | | | |
| | | | | | methyl | | | | | | | | | | |
| 96 | CR³ | CR⁴ | H | H | H | Cl | H | H | H | CF₃ | H | bond | — | cyclopropyl | 3.72* |
| 97 | CR³ | CR⁴ | H | H | 2- | H | H | H | H | Br | H | bond | — | cyclopropyl | 1.49* |
| | | | | | oxopyrrolidin- | | | | | | | | | | |
| | | | | | 1-yl | | | | | | | | | | |
| 98 | CR³ | CR⁴ | H | H | H | 2- | H | H | H | Br | H | bond | — | cyclopropyl | 1.57* |
| | | | | | | oxopyrrolidin- | | | | | | | | | |
| | | | | | | 1-yl | | | | | | | | | |
| 99 | CR³ | CR⁴ | H | H | H | 2- | H | H | H | Cl | H | bond | — | cyclopropyl | 1.62* |
| | | | | | | furoylamino | | | | | | | | | |
| 100 | CR³ | CR⁴ | H | H | NHCOOtBu | H | H | H | H | Cl | H | bond | — | cyclopropyl | 2.12* |
| 101 | CR³ | CR⁴ | H | H | hydroxyl | COMe | H | H | H | Cl | H | bond | — | cyclopropyl | 1.65* |
| 102 | CR³ | CR⁴ | H | H | [(dimethylamino)- | H | H | H | H | Cl | H | bond | — | cyclopropyl | 2.08* |
| | | | | | sulphonyl]oxy | | | | | | | | | | |
| 103 | CR³ | CR⁴ | H | H | —OCH₂CON(CH₃)— | | H | H | H | Cl | H | bond | | cyclopropyl | 1.41* |
| 104 | CR³ | CR⁴ | H | H | (difluoromethyl)- | H | H | H | H | Br | H | bond | — | cyclopropyl | 2.99* |
| | | | | | thio | | | | | | | | | | |
| 105 | CR³ | CR⁴ | H | H | hydroxy | F | H | H | H | Cl | H | bond | — | cyclopropyl | 1.25* |
| 106 | CR³ | CR⁴ | H | H | pentanoyloxy | H | H | H | H | Cl | H | bond | — | cyclopropyl | 3.68* |
| 107 | CR³ | CR⁴ | H | H | 3-methyl-2,5- | H | H | H | H | Cl | H | bond | — | cyclopropyl | 1.29* |
| | | | | | dioxo- | | | | | | | | | | |
| | | | | | imidazolidin- | | | | | | | | | | |
| | | | | | 1-yl | | | | | | | | | | |
| 108 | CR³ | CR⁴ | H | H | 1,1,2,2- | H | H | H | H | Cl | H | bond | — | cyclopropyl | 2.65* |
| | | | | | tetrafluoroethoxy | | | | | | | | | | |
| 109 | CR³ | CR⁴ | H | H | 4-(1- | H | H | H | H | Cl | H | bond | — | cyclopropyl | 2.79* |
| | | | | | methylethyl)-2- | | | | | | | | | | |
| | | | | | oxo-1,3- | | | | | | | | | | |
| | | | | | oxazolidin-3-yl | | | | | | | | | | |
| 110 | CR³ | CR⁴ | H | H | (2-methoxy-1- | H | H | H | H | Cl | H | bond | — | cyclopropyl | 2.03* |
| | | | | | methylethyl) | | | | | | | | | | |
| | | | | | amino | | | | | | | | | | |
| 111 | CR³ | CR⁴ | H | H | NHCOisoPr | H | H | H | H | Cl | H | bond | — | cyclopropyl | 1.43* |
| 112 | CR³ | CR⁴ | H | H | hydroxyl | H | Me | H | H | Br | H | bond | — | cyclopropyl | 1.21* |
| 113 | CR³ | CR⁴ | H | H | Me | NHCOMe | H | H | H | Br | H | bond | — | cyclopropyl | 1.35* |
| 114 | CR³ | CR⁴ | H | H | hydroxyl | H | H | H | H | Br | H | bond | — | cyclopropyl | 1.17* |
| 115 | CR³ | CR⁴ | H | H | —N(CH₃)COCH₂O— | | H | H | H | Br | H | bond | — | cyclopropyl | 1.61* |
| 116 | CR³ | CR⁴ | H | H | hydroxyl | Me | H | H | H | Br | H | bond | — | cyclopropyl | 1.35* |
| 117 | CR³ | CR⁴ | H | H | 2-piperidin-1- | H | H | H | H | Br | H | bond | — | cyclopropyl | 1.07* |
| | | | | | ylethoxy | | | | | | | | | | |
| 118 | CR³ | CR⁴ | H | H | 4-methyl-2- | H | H | H | H | Br | H | methylene | — | cyclopropyl | 1.72* |
| | | | | | oxo-1,3- | | | | | | | | | | |
| | | | | | oxazolidin-3-yl | | | | | | | | | | |
| 119 | CR³ | CR⁴ | H | H | H | SMe | H | H | H | Cl | H | methylene | — | cyclopropyl | 2.27* |
| 120 | CR³ | CR⁴ | H | H | cyclopropyl(tri- | H | H | H | H | Cl | H | bond | — | cyclopropyl | 2.54* |
| | | | | | fluoroacetyl)amino | | | | | | | | | | |
| 121 | CR³ | CR⁴ | H | H | H | SMe | H | H | H | Br | H | bond | — | cyclopropyl | 2.44* |
| 122 | CR³ | CR⁴ | H | H | H | benzoylamino | H | H | H | Br | H | bond | — | cyclopropyl | 2.26* |
| 123 | CR³ | CR⁴ | H | H | H | (methylcarbamoyl) | H | H | H | Cl | H | bond | — | cyclopropyl | 1.46* |
| | | | | | | oxy | | | | | | | | | |
| 124 | CR³ | CR⁴ | H | H | —CH₂CH₂N(COCH₃)— | | H | H | H | Cl | H | bond | | cyclopropyl | 1.56* |
| 125 | CR³ | CR⁴ | H | H | F | H | H | H | H | Br | H | methylene | — | cyclopropyl | 2.06* |
| 126 | CR³ | CR⁴ | H | H | H | 2- | H | H | H | CN | H | bond | — | cyclopropyl | 1.98* |
| | | | | | | oxopyrrolidin- | | | | | | | | | |
| | | | | | | 1-yl | | | | | | | | | |
| 127 | CR³ | CR⁴ | H | H | H | Cl | H | H | H | Br | H | methylene | — | cyclopropyl | 3.1* |
| 128 | CR³ | CR⁴ | H | H | H | [(1-methyl- | H | H | H | Cl | H | bond | — | cyclopropyl | 1.77* |
| | | | | | | cyclopropyl) | | | | | | | | | |
| | | | | | | carbonyl]amino | | | | | | | | | |
| 129 | CR³ | CR⁴ | H | H | morpholin-4-yl | — | H | H | H | Cl | H | bond | — | cyclopropyl | 1.02* |
| 130 | CR³ | CR⁴ | H | H | H | (3-fluoro-2,2- | H | H | H | Br | H | bond | — | cyclopropyl | 1.89* |
| | | | | | | dimethylpropanoyl) | | | | | | | | | |
| | | | | | | amino | | | | | | | | | |
| 131 | CR³ | CR⁴ | H | H | (3-chloro-2,2- | H | H | H | H | Br | H | bond | — | cyclopropyl | 1.97* |
| | | | | | dimethylpropanoyl)- | | | | | | | | | | |
| | | | | | amino | | | | | | | | | | |
| 132 | CR³ | CR⁴ | H | H | cyclopropylamino | — | H | H | H | Br | H | bond | — | cyclopropyl | 1.15* |
| 133 | CR³ | CR⁴ | H | H | 2- | H | H | H | H | Br | H | bond | — | cyclopropyl | 0.82* |
| | | | | | (diethylamino)- | | | | | | | | | | |
| | | | | | ethoxy | | | | | | | | | | |
| 134 | CR³ | CR⁴ | H | H | morpholin-4- | H | H | H | H | Cl | H | bond | — | cyclopropyl | 0.84* |
| | | | | | ylmethyl | | | | | | | | | | |
| 135 | CR³ | CR⁴ | H | H | —CH₂OCH₂O— | | H | H | H | Cl | H | bond | | cyclopropyl | 1.59* |
| 136 | CR³ | CR⁴ | H | H | OMe | Cl | H | H | H | Cl | H | bond | — | cyclopropyl | 2.08* |
| 137 | CR³ | CR⁴ | H | H | acetyl(cyclohexyl)- | H | H | H | H | Br | H | bond | — | cyclopropyl | 2.5* |
| | | | | | amino | | | | | | | | | | |
| 138 | CR³ | CR⁴ | H | H | —OC(CH₃)═N— | | H | H | H | Cl | H | bond | | cyclopropyl | 1.57* |
| 139 | CR³ | CR⁴ | H | H | hydroxyl | NHCOMe | H | H | H | Cl | H | bond | — | cyclopropyl | 1* |
| 140 | CR³ | CR⁴ | H | H | 2,5- | H | H | H | H | Cl | H | bond | — | cyclopropyl | 1.32* |
| | | | | | dioxopyrrolidin- | | | | | | | | | | |
| | | | | | 1-yl | | | | | | | | | | |
| 141 | CR³ | CR⁴ | H | OMe | OMe | OMe | H | H | H | CN | H | bond | — | cyclopropyl | 2.14* |
| 142 | CR³ | CR⁴ | H | H | —CH₂CH₂CH₂CONH— | | H | H | H | Br | H | bond | | cyclopropyl | 1.48* |
| 143 | CR³ | CR⁴ | H | H | (2,2- | H | H | H | H | Cl | H | bond | — | cyclopropyl | 1.96* |
| | | | | | dimethylpropanoyl) | | | | | | | | | | |
| | | | | | (methyl)amino | | | | | | | | | | |
| 144 | CR³ | CR⁴ | H | H | 4,4-dimethyl-2,5- | H | H | H | H | Cl | H | bond | — | cyclopropyl | 1.51* |
| | | | | | dioxoimidazolidin- | | | | | | | | | | |
| | | | | | 1-yl | | | | | | | | | | |
| 145 | CR³ | CR⁴ | H | H | H | 2-oxopyrrol- | H | Me | H | Cl | H | bond | — | cyclopropyl | 1.39* |
| | | | | | | idin-1-yl | | | | | | | | | |
| 146 | CR³ | CR⁴ | H | H | 2,3-dimethyl-5- | H | H | H | H | Cl | H | bond | — | cyclopropyl | 0.96* |
| | | | | | oxo-2,5-dihydro- | | | | | | | | | | |
| | | | | | 1H-pyrazol-1-yl | | | | | | | | | | |
| 147 | CR³ | CR⁴ | H | H | H | 5-thioxo-4,5- | H | H | H | Cl | H | bond | — | cyclopropyl | 1.48* |
| | | | | | | dihydro-1H- | | | | | | | | | |
| | | | | | | tetrazol-1-yl | | | | | | | | | |
| 148 | CR³ | CR⁴ | H | H | 1,1,2,2- | H | H | H | H | Cl | H | methylene | — | cyclopropyl | 2.82* |
| | | | | | tetrafluoroethoxy | | | | | | | | | | |
| 149 | CR³ | CR⁴ | H | H | 1,1,2,2- | H | H | H | H | Br | H | bond | — | cyclopropyl | 2.95* |
| | | | | | tetrafluoroethoxy | | | | | | | | | | |
| 150 | CR³ | CR⁴ | Me | H | H | (butoxycarbonyl) | H | H | H | Cl | H | bond | — | cyclopropyl | 2.15* |
| | | | | | | amino | | | | | | | | | |
| 151 | CR³ | CR⁴ | H | H | H | 3-methyl-2- | H | H | H | Cl | H | bond | — | cyclopropyl | 1.49* |
| | | | | | | oxoimidazolidin- | | | | | | | | | |
| | | | | | | 1-yl | | | | | | | | | |
| 152 | CR³ | CR⁴ | H | H | tert-butylthio | H | H | H | H | Cl | H | bond | — | cyclopropyl | 3.73* |
| 153 | CR³ | CR⁴ | H | H | (methoxycarbonyl)- | H | H | H | H | Cl | H | methylene | — | cyclopropyl | 1.44* |
| | | | | | amino | | | | | | | | | | |
| 154 | CR³ | CR⁴ | H | H | 2- | H | H | H | H | Cl | H | methylene | — | cyclopropyl | 1.5* |
| | | | | | oxopyrrolidin-1-yl | | | | | | | | | | |
| 155 | CR³ | CR⁴ | H | H | H | NHCOMe | H | H | H | Br | H | bond | — | cyclopropyl | 1.86* |
| 156 | CR³ | CR⁴ | H | H | 2,5-dioxo-2,5-di- | H | H | H | H | Cl | H | bond | — | cyclopropyl | 1.58* |
| | | | | | hydro-1H-pyrrol-1-yl | | | | | | | | | | |
| 157 | CR³ | CR⁴ | H | H | 2- | H | H | H | H | CN | H | bond | — | cyclopropyl | 1.89* |
| | | | | | oxopyrrolidin-1-yl | | | | | | | | | | |
| 158 | CR³ | CR⁴ | H | H | acetyl(methyl)- | H | H | H | H | CN | H | bond | — | cyclopropyl | 1.69* |
| | | | | | amino | | | | | | | | | | |
| 159 | CR³ | CR⁴ | H | H | (methoxycarbonyl)- | H | H | H | H | CN | H | bond | — | cyclopropyl | 1.91* |
| | | | | | amino | | | | | | | | | | |
| 160 | CR³ | CR⁴ | H | H | 2-oxo-1,3- | H | H | H | H | Cl | H | methylene | — | cyclopropyl | 1.39* |
| | | | | | oxazolidin-3-yl | | | | | | | | | | |
| 161 | CR³ | CR⁴ | H | H | prop-2-yn-1- | H | H | H | H | Cl | H | bond | — | cyclopropyl | 2.21* |
| | | | | | ylsulphonyl | | | | | | | | | | |
| 162 | CR³ | CR⁴ | H | H | CH₂NHCOOtBu | H | H | H | H | Cl | H | methylene | — | cyclopropyl | 2.21* |
| 163 | CR³ | CR⁴ | H | H | H | phenylthio | H | H | H | Cl | H | bond | — | cyclopropyl | 3.61* |
| 164 | CR³ | CR⁴ | H | H | H | formamido | H | H | H | Cl | H | bond | — | cyclopropyl | 1.11* |
| 165 | CR³ | CR⁴ | H | H | H | pyrrolidin-1- | H | H | H | Cl | H | bond | — | cyclopropyl | 2.46** |
| | | | | | | ylsulphonyl | | | | | | | | | |
| 166 | CR³ | CR⁴ | H | H | H | H | H | H | H | Cl | H | —CH(CH₃)— | — | cyclopropyl | 2.14* |
| 167 | CR³ | CR⁴ | H | H | F | H | H | H | H | Cl | H | —CH(CH₃)— | — | cyclopropyl | 2.27* |
| 168 | CR³ | CR⁴ | H | H | (methoxycarbonyl)- | H | H | H | H | Cl | H | —CH(CH₃)— | — | cyclopropyl | 1.64* |
| | | | | | amino | | | | | | | | | | |
| 169 | CR³ | CR⁴ | H | H | H | 2-oxopyrrol- | H | H | H | Cl | H | —CH(CH₃)— | — | cyclopropyl | 1.83* |
| | | | | | | idin-1-yl | | | | | | | | | |
| 170 | CR³ | CR⁴ | H | H | H | SMe | H | H | H | Cl | H | —CH(CH₃)— | — | cyclopropyl | 2.77* |
| 171 | CR³ | CR⁴ | H | H | H | hydroxy | H | H | H | Cl | H | —CH(CH₃)— | — | cyclopropyl | 1.62* |
| 172 | CR³ | CR⁴ | H | H | H | NHCOMe | H | H | H | Cl | H | methylene | — | cyclopropyl | 1.45* |
| 173 | CR³ | CR⁴ | H | H | (methoxycarbonyl)- | H | H | H | H | Br | H | bond | — | cyclopropyl | 1.42* |
| | | | | | amino | | | | | | | | | | |
| 174 | CR³ | CR⁴ | H | H | H | 1,1,2,2- | H | H | H | Br | H | bond | — | cyclopropyl | 3.17* |
| | | | | | | tetrafluoroethoxy | | | | | | | | | |
| 175 | CR³ | CR⁴ | H | H | 2-oxo-1,3- | H | H | H | H | Br | H | bond | — | cyclopropyl | 1.37* |
| | | | | | oxazolidin-3-yl | | | | | | | | | | |
| 176 | CR³ | CR⁴ | H | H | 2,5- | H | H | H | H | Cl | H | bond | — | cyclopropyl | 0.61* |
| | | | | | dioxoimidazol- | | | | | | | | | | |
| | | | | | idin-4-yl | | | | | | | | | | |
| 177 | CR³ | CR⁴ | H | H | hydroxy | H | Me | H | H | Cl | H | bond | — | cyclopropyl | 1.09* |
| 178 | CR³ | CR⁴ | H | H | H | benzoylamino | H | H | H | Cl | H | bond | — | cyclopropyl | 2.11* |
| 179 | CR³ | CR⁴ | H | H | —N(CH₃)COCH₂O— | | H | H | H | Cl | H | bond | | cyclopropyl | 1.47* |
| 180 | CR³ | CR⁴ | H | H | hydroxy | Me | H | H | H | Cl | H | bond | — | cyclopropyl | 1.09* |
| 181 | CR³ | CR⁴ | H | H | —CH₂CH₂CH₂CONH— | | H | H | H | Cl | H | bond | | cyclopropyl | 1.41* |
| 182 | CR³ | CR⁴ | H | H | H | 1,1,2,2- | H | H | H | Cl | H | bond | — | cyclopropyl | 3.03* |
| | | | | | | terafluoroethoxy | | | | | | | | | |
| 183 | CR³ | CR⁴ | H | H | ethoxy | H | H | H | H | Cl | H | bond | — | cyclopropyl | 1.76* |
| 184 | CR³ | CR⁴ | H | H | H | (trifluoromethyl)- | H | H | H | Cl | H | bond | — | cyclopropyl | 3.55* |
| | | | | | | amino | | | | | | | | | |
| 185 | CR³ | CR⁴ | H | H | 1,1,2,2- | H | Me | H | H | Cl | H | bond | — | cyclopropyl | 2.5* |
| | | | | | tetrafluoroethoxy | | | | | | | | | | |
| 186 | CR³ | N | H | H | cyclopropylamino | — | H | H | H | Cl | H | bond | — | cyclopropyl | 0.92* |
| 187 | CR³ | CR⁴ | H | H | hydroxyl | cyclopentyl | H | H | H | Cl | H | bond | — | cyclopropyl | 1.86* |
| 188 | CR³ | CR⁴ | H | H | 2-methoxy-1- | H | H | H | H | Cl | H | bond | — | cyclopropyl | 1.73* |
| | | | | | methylethoxy | | | | | | | | | | |
| 189 | CR³ | CR⁴ | H | H | H | H | H | H | H | Br | H | methylene | — | cyclopropyl | 1.93* |
| 190 | CR³ | CR⁴ | H | OMe | OMe | OMe | H | H | H | Br | H | methylene | — | cyclopropyl | 1.88* |
| 191 | CR³ | N | H | H | OMe | — | H | H | H | Br | H | methylene | — | cyclopropyl | 1.63* |
| 192 | CR³ | CR⁴ | H | H | H | SO₂NMe₂ | H | H | H | Br | H | methylene | — | cyclopropyl | 2.48* |
| 193 | CR³ | CR⁴ | H | H | H | (methoxycarbonyl)- | H | H | H | Cl | H | bond | — | cyclopropyl | 1.44* |
| | | | | | | amino | | | | | | | | | |
| 194 | CR³ | CR⁴ | OMe | H | H | 2-oxopyrrol- | H | H | H | Cl | H | bond | — | cyclopropyl | 1.54* |
| | | | | | | idin-1-yl | | | | | | | | | |
| 195 | CR³ | CR⁴ | H | H | H | isopropoxy | H | H | H | Cl | H | bond | — | cyclopropyl | 2.45* |
| 196 | CR³ | CR⁴ | H | H | H | methoxycarbonyl | H | H | H | Cl | H | bond | — | cyclopropyl | 1.9* |
| 197 | CR³ | CR⁴ | H | H | H | 2-thienyl | H | H | H | Cl | H | bond | — | cyclopropyl | 2.9* |
| 198 | CR³ | CR⁴ | H | H | 2-piperidin-1- | H | H | H | H | Cl | H | bond | — | cyclopropyl | 0.74* |
| | | | | | ylethoxy | | | | | | | | | | |
| 199 | CR³ | CR⁴ | H | H | H | OMe | H | H | H | CF₃ | H | methylene | — | cyclopropyl | 2.91* |
| 200 | CR³ | CR⁴ | H | H | methylsulphonyl | H | H | H | H | CF₃ | H | methylene | — | cyclopropyl | 2.63* |
| 201 | CR³ | CR⁴ | H | H | NHCOtBu | H | H | H | H | CF₃ | H | methylene | — | cyclopropyl | 2.6* |
| 202 | CR³ | CR⁴ | H | H | acetyl(methyl)- | H | H | H | H | CF₃ | H | methylene | — | cyclopropyl | 2.13* |
| | | | | | amino | | | | | | | | | | |
| 203 | CR³ | CR⁴ | H | H | (trifluoroacetyl)- | H | H | H | H | CF₃ | H | methylene | — | cyclopropyl | 2.84* |
| | | | | | amino | | | | | | | | | | |
| 204 | CR³ | CR⁴ | H | H | H | NHCOMe | H | H | H | CF₃ | H | methylene | — | cyclopropyl | 1.9* |
| 205 | CR³ | CR⁴ | H | H | H | NHCOtBu | H | H | H | CF₃ | H | methylene | — | cyclopropyl | 2.77* |
| 206 | CR³ | CR⁴ | H | H | dimethylcarbamoyl | H | H | H | H | CF₃ | H | methylene | — | cyclopropyl | 2.13* |
| 207 | CR³ | CR⁴ | H | H | tert-butylcarbamoyl | H | H | H | H | CF₃ | H | methylene | — | cyclopropyl | 3.07* |
| 208 | CR³ | CR⁴ | H | H | H | SMe | H | H | H | CF₃ | H | methylene | — | cyclopropyl | 3.41* |
| 209 | CR³ | CR⁴ | H | H | H | piperidin-1- | H | H | H | CF₃ | H | methylene | — | cyclopropyl | 3.81* |
| | | | | | | ylsulphonyl | | | | | | | | | |
| 210 | CR³ | CR⁴ | H | H | 3,3-dimethyl-2- | H | H | H | H | CF₃ | H | methylene | — | cyclopropyl | 3.41* |
| | | | | | oxobutyl | | | | | | | | | | |
| 211 | CR³ | CR⁴ | H | H | H | COMe | H | H | H | CF₃ | H | methylene | — | cyclopropyl | 2.77* |
| 212 | CR³ | CR⁴ | H | H | 2-oxo-1,3- | H | H | H | H | CF₃ | H | methylene | — | cyclopropyl | 2.09* |
| | | | | | oxazolidin-3-yl | | | | | | | | | | |
| 213 | CR³ | CR⁴ | H | H | 2-oxopyrrolidin- | H | H | H | H | CF₃ | H | methylene | — | cyclopropyl | 2.16* |
| | | | | | 1-yl | | | | | | | | | | |
| 214 | CR³ | CR⁴ | H | H | H | 2- | H | H | H | CF₃ | H | methylene | — | cyclopropyl | 2.26* |
| | | | | | | oxopyrrolidin- | | | | | | | | | |
| | | | | | | 1-yl | | | | | | | | | |
| 215 | CR³ | CR⁴ | H | H | H | Cl | H | H | H | CF₃ | H | methylene | — | cyclopropyl | 3.9* |
| 216 | CR³ | CR⁴ | H | H | H | SO₂NMe₂ | H | H | H | CF₃ | H | methylene | — | cyclopropyl | 3.1* |
| 217 | CR³ | CR⁴ | H | OMe | OMe | OMe | H | H | H | CF₃ | H | methylene | — | cyclopropyl | 2.57* |
| 218 | CR³ | CR⁴ | H | H | [(1-methylcyclopropyl) | H | H | H | H | CF₃ | H | methylene | — | cyclopropyl | 2.43* |
| | | | | | carbonyl]-amino | | | | | | | | | | |
| 219 | CR³ | CR⁴ | H | H | methylamino | H | H | H | H | CF₃ | H | methylene | — | cyclopropyl | 3.07* |
| 220 | CR³ | CR⁴ | H | H | H | 2-oxopyrrolidin- | H | H | H | Br | H | methylene | — | cyclopropyl | 1.67* |
| | | | | | | 1-yl | | | | | | | | | |
| 221 | CR³ | CR⁴ | H | H | H | NHCOisoPr | H | H | H | Br | H | bond | — | cyclopropyl | 1.66* |
| 222 | CR³ | CR⁴ | H | H | H | (difluoromethyl)- | H | H | H | Cl | H | bond | — | cyclopropyl | 2.84* |
| | | | | | | thio | | | | | | | | | |
| 223 | CR³ | CR⁴ | H | H | H | NHCOisoPr | H | H | H | Cl | H | methylene | — | cyclopropyl | 1.73* |
| 224 | CR³ | CR⁴ | H | H | H | NHCOtertBu | H | H | H | Br | H | bond | — | cyclopropyl | 2.12* |
| 225 | CR³ | CR⁴ | H | H | NHCOMe | H | H | H | H | Cl | H | bond | — | cyclopropyl | 1.27* |
| 226 | CR³ | CR⁴ | H | H | NHCOMe | Cl | H | H | H | Cl | H | bond | — | cyclopropyl | 1.61* |
| 227 | CR³ | CR⁴ | H | H | acetyl(ethyl)amino | H | H | H | H | Cl | H | bond | — | cyclopropyl | 1.64* |
| 228 | CR³ | CR⁴ | H | H | (trifluoroacetyl)- | H | H | H | H | Cl | H | bond | — | cyclopropyl | 1.9* |
| | | | | | amino | | | | | | | | | | |
| 229 | CR³ | N | H | H | acetylamino | — | H | H | H | Cl | H | bond | — | cyclopropyl | 1.29* |
| 230 | CR³ | CR⁴ | H | H | H | acryloylamino | H | H | H | Cl | H | bond | — | cyclopropyl | 1.49* |
| 231 | CR³ | CR⁴ | H | H | H | propionylamino | H | H | H | Cl | H | bond | — | cyclopropyl | 1.49* |
| 232 | CR³ | CR⁴ | H | H | H | methacryloyl | H | H | H | Cl | H | bond | — | cyclopropyl | 1.63* |
| | | | | | | amino | | | | | | | | | |
| 233 | CR³ | CR⁴ | H | H | dimethylcarbamoyl | H | H | H | H | Cl | H | bond | — | cyclopropyl | 1.51* |
| 234 | CR³ | CR⁴ | H | H | morpholin-4- | H | H | H | H | Cl | H | bond | — | cyclopropyl | 1.51* |
| | | | | | ylcarbamoyl | | | | | | | | | | |
| 235 | CR³ | CR⁴ | H | H | methylcarbomoyl | H | H | H | H | Cl | H | bond | — | cyclopropyl | 1.39* |
| 236 | CR³ | CR⁴ | H | H | allylcarbamoyl | H | H | H | H | Cl | H | bond | — | cyclopropyl | 1.66* |
| 237 | CR³ | CR⁴ | H | H | tert- | H | H | H | H | Cl | H | bond | — | cyclopropyl | 2.09* |
| | | | | | butylcarbamoyl | | | | | | | | | | |
| 238 | CR³ | CR⁴ | H | H | H | methylcarbamoyl | H | H | H | Cl | H | bond | — | cyclopropyl | 1.32* |
| 239 | CR³ | CR⁴ | H | H | Cl | methylsulphonyl | H | H | H | Cl | H | bond | — | cyclopropyl | 2.11* |
| 240 | CR³ | CR⁴ | H | H | (methylsulphonyl) | OMe | H | H | H | Cl | H | bond | — | cyclopropyl | 1.51* |
| | | | | | amino | | | | | | | | | | |
| 241 | CR³ | CR⁴ | H | H | (methylsulphonyl)- | H | H | H | H | Cl | H | bond | — | cyclopropyl | 1.41* |
| | | | | | amino | | | | | | | | | | |
| 242 | CR³ | CR⁴ | H | H | H | (methylsulphon- | H | H | H | Cl | H | bond | — | cyclopropyl | 1.47* |
| | | | | | | yl)amino | | | | | | | | | |
| 243 | CR³ | CR⁴ | H | H | H | (methylsulphon- | H | H | H | Cl | H | bond | — | cyclopropyl | 1.46* |
| | | | | | | yl)methyl | | | | | | | | | |
| 244 | CR³ | CR⁴ | H | H | H | [(dimethylamino)- | H | H | H | Cl | H | bond | — | cyclopropyl | 2.26* |
| | | | | | | sulphonyl]oxy | | | | | | | | | |
| 245 | CR³ | CR⁴ | H | H | —CH₂SO₂CH₂— | | H | H | H | Cl | H | bond | | cyclopropyl | 1.51* |
| 246 | CR³ | CR⁴ | H | H | H | piperidin-1- | H | H | H | Cl | H | bond | — | cyclopropyl | 2.8* |
| | | | | | | ylsulphonyl | | | | | | | | | |
| 247 | CR³ | CR⁴ | H | H | 2-ethoxy-2- | H | H | H | H | Cl | H | bond | — | cyclopropyl | 1.99* |
| | | | | | oxoethyl | | | | | | | | | | |
| 248 | CR³ | CR⁴ | H | H | cyanoacetyl | H | H | H | H | Cl | H | bond | — | cyclopropyl | 2.09* |
| 249 | CR³ | CR⁴ | H | H | Cl | COMe | H | H | H | Cl | H | bond | — | cyclopropyl | 2.43* |
| 250 | CR³ | CR⁴ | H | H | hydroxy | propionyl | H | H | H | Cl | H | bond | — | cyclopropyl | 1.99* |
| 251 | CR³ | CR⁴ | H | H | 1,3-thiazol-2-yl | H | H | H | H | Br | H | bond | — | cyclopropyl | 2.49* |
| 252 | CR³ | CR⁴ | H | H | H | methylsulphonyl | H | H | H | Cl | H | bond | — | cyclopropyl | 1.63* |
| 253 | CR³ | CR⁴ | H | H | 1,3-thiazol-2-yl | H | H | H | H | Cl | H | bond | — | cyclopropyl | 2.32* |
| 254 | CR³ | CR⁴ | H | H | H | methylsulphonyl | H | H | H | Br | H | bond | — | cyclopropyl | 1.76* |
| 255 | CR³ | CR⁴ | H | H | H | 2-oxopyrrolidin- | H | H | H | CF₃ | H | bond | — | cyclopropyl | 2.23* |
| | | | | | | 1-yl | | | | | | | | | |
| 256 | CR³ | CR⁴ | H | H | Cl | OMe | H | H | H | Cl | H | bond | — | cyclopropyl | 2.53* |
| 257 | CR³ | CR⁴ | H | H | propionyl | H | H | H | H | Cl | H | bond | — | cyclopropyl | 2.34* |
| 258 | CR³ | CR⁴ | H | H | —COCH₂CH₂— | | H | H | H | Cl | H | bond | — | cyclopropyl | 1.94* |
| 259 | CR³ | CR⁴ | H | H | F | OMe | H | H | H | Cl | H | bond | — | cyclopropyl | 1.9* |
| 260 | CR³ | CR⁴ | H | H | ethyl(methoxy- | H | H | H | H | Cl | H | bond | — | cyclopropyl | 1.95* |
| | | | | | carbonyl)amino | | | | | | | | | | |
| 261 | CR³ | CR⁴ | H | H | H | (morpholin-4- | H | H | H | Cl | H | bond | — | cyclopropyl | 1.62* |
| | | | | | | 4-ylsulphonyl)- | | | | | | | | | |
| | | | | | | methyl | | | | | | | | | |
| 262 | CR³ | CR⁴ | H | H | H | (piperidin-1-yl- | H | H | H | Cl | H | bond | — | cyclopropyl | 2.24* |
| | | | | | | sulphonyl) | | | | | | | | | |
| | | | | | | methyl | | | | | | | | | |
| 263 | CR³ | CR⁴ | H | 2- | H | H | H | H | H | Cl | H | bond | HCl | cyclopropyl | 1.46* |
| | | | | oxopyrro- | | | | | | | | | | | |
| | | | | lidin-1-yl | | | | | | | | | | | |
| 264 | CR³ | CR⁴ | H | H | H | isopropylthio | H | H | H | Cl | H | bond | — | cyclopropyl | 3.18* |
| 265 | CR³ | CR⁴ | H | H | F | 2-oxopyrrolidin- | H | H | H | CF₃ | H | bond | — | cyclopropyl | 2.33* |
| | | | | | | 1-yl | | | | | | | | | |
| 266 | CR³ | CR⁴ | H | H | H | SMe | H | H | H | CF₃ | H | bond | — | cyclopropyl | 3.29*; |
| | | | | | | | | | | | | | | | 3.49** |
| 267 | CR³ | CR⁴ | H | H | H | 4-methyl-2-oxo- | H | H | H | Cl | H | bond | — | cyclopropyl | 1.57* |
| | | | | | | 1,3-oxazolidin- | | | | | | | | | |
| | | | | | | 3-yl | | | | | | | | | |
| 268 | CR³ | CR⁴ | H | H | H | methoxymethyl | H | H | H | Cl | H | bond | — | cyclopropyl | 1.67* |
| 269 | CR³ | CR⁴ | H | H | H | methoxymethyl | H | H | H | Br | H | bond | — | cyclopropyl | 1.8* |
| 270 | CR³ | CR⁴ | H | H | H | 2,2,2-trifluoro- | H | H | H | Cl | H | bond | — | cyclopropyl | 2.69* |
| | | | | | | 1-methoxyethyl | | | | | | | | | |
| 271 | CR³ | CR⁴ | H | H | chloro- | H | H | H | H | Cl | H | bond | — | cyclopropyl | |
| | | | | carbonyl | | | | | | | | | | | |
| 272 | CR³ | CR⁴ | H | H | amino | H | H | H | H | Cl | H | bond | — | cyclopropyl | 0.45*; |
| | | | | | | | | | | | | | | | 0.48** |
| 273 | CR³ | CR⁴ | Me | H | H | H | Me | H | H | Cl | H | bond | — | cyclopropyl | 1.59* |
| 274 | CR³ | CR⁴ | H | Me | H | Me | H | H | H | Cl | H | bond | — | cyclopropyl | 2.13* |
| 275 | CR³ | CR⁴ | H | H | H | H | F | H | H | Cl | H | bond | — | cyclopropyl | 2.09* |
| 276 | CR³ | CR⁴ | H | H | H | H | CF₃ | H | H | Cl | H | bond | — | cyclopropyl | 2.58* |
| 277 | CR³ | CR⁴ | H | H | CF₃ | H | H | H | H | Cl | H | bond | — | cyclopropyl | 3.14* |
| 278 | CR³ | CR⁴ | H | H | H | CN | H | H | H | Cl | H | bond | — | cyclopropyl | 2.13* |
| 279 | CR³ | CR⁴ | H | H | OMe | OMe | H | H | H | Cl | H | bond | — | cyclopropyl | 1.47* |
| 280 | CR³ | CR⁴ | H | H | NHCOMe | OMe | H | H | H | Cl | H | bond | — | cyclopropyl | 1.18* |
| 281 | CR³ | CR⁴ | H | H | Br | H | H | H | H | Cl | H | bond | — | cyclopropyl | 2.55* |
| 282 | CR³ | CR⁴ | H | H | H | H | OMe | H | H | Cl | H | bond | — | cyclopropyl | 1.85* |
| 283 | CR³ | CR⁴ | H | H | H | OMe | H | H | H | Cl | H | bond | — | cyclopropyl | 1.8* |
| 284 | CR³ | CR⁴ | H | H | OMe | H | H | H | H | Cl | H | bond | — | cyclopropyl | 1.47* |
| 285 | CR³ | CR⁴ | H | H | Cl | H | H | H | H | Cl | H | bond | — | cyclopropyl | 2.54* |
| 286 | CR³ | CR⁴ | H | H | 1,3-thiazol-4-yl | H | H | H | H | Cl | H | bond | — | cyclopropyl | 1.9* |
| 287 | CR³ | CR⁴ | H | H | 2-methoxy-2- | H | H | H | H | Cl | H | bond | — | cyclopropyl | 1.85* |
| | | | | | oxoethyl | | | | | | | | | | |
| 288 | CR³ | CR⁴ | H | H | difluoromethoxy | H | H | H | H | Cl | H | bond | — | cyclopropyl | 2.11* |
| 289 | CR³ | CR⁴ | H | H | —OCF₂CF₂O— | | H | H | H | Cl | H | bond | — | cyclopropyl | 3.76* |
| 290 | CR³ | CR⁴ | H | H | H | 2-methoxy-2- | H | H | H | Cl | H | bond | — | cyclopropyl | 1.73* |
| | | | | | | oxoethyl | | | | | | | | | |
| 291 | CR³ | CR⁴ | H | Cl | H | Cl | H | H | H | Cl | H | bond | — | cyclopropyl | 4.04* |
| 292 | CR³ | CR⁴ | H | H | H | ethoxycarbonyl | H | H | H | Cl | H | bond | — | cyclopropyl | 2.26* |
| 293 | CR³ | CR⁴ | H | H | H | ethoxy | H | H | H | Cl | H | bond | — | cyclopropyl | 2.06* |
| 294 | CR³ | CR⁴ | H | H | hydroxyl | H | F | H | H | Cl | H | bond | — | cyclopropyl | 1.26* |
| 295 | CR³ | CR⁴ | H | H | methylenebis(oxy) | | H | H | H | Cl | H | bond | — | cyclopropyl | 1.59* |
| 296 | CR³ | CR⁴ | H | H | trifluoromethoxy | Cl | H | H | H | Cl | H | bond | — | cyclopropyl | 3.9* |
| 297 | CR³ | CR⁴ | H | H | H | 1,3-thiazol-2-yl | H | H | H | Cl | H | bond | — | cyclopropyl | 2.21* |
| 298 | CR³ | CR⁴ | Cl | H | Cl | 2-oxopyrrolidin- | H | H | H | Cl | H | bond | — | cyclopropyl | 2.59* |
| | | | | | | 1-yl | | | | | | | | | |
| 299 | CR³ | CR⁴ | H | H | cyclopentyloxy | H | H | H | H | Cl | H | bond | — | cyclopropyl | 2.48* |
| 300 | CR³ | CR⁴ | H | H | H | trifluoromethoxy | H | H | H | Cl | H | bond | — | cyclopropyl | 3.18* |
| 301 | CR³ | CR⁴ | H | H | H | trifluoromethoxy | H | H | H | Br | H | bond | — | cyclopropyl | 3.68* |
| 302 | CR³ | CR⁴ | H | H | H | (isopropoxy- | H | H | H | Cl | H | bond | — | cyclopropyl | 1.99* |
| | | | | | | carbonyl)amino | | | | | | | | | |
| 303 | CR³ | CR⁴ | H | H | ethyl(methoxy- | H | H | H | H | Br | H | bond | — | cyclopropyl | 2.06* |
| | | | | | carbonyl)amino | | | | | | | | | | |
| 304 | CR³ | CR⁴ | H | H | H | propionyl | H | H | H | Cl | H | bond | — | cyclopropyl | 2.25* |
| 305 | CR³ | CR⁴ | H | H | H | propionyl | H | H | H | Br | H | bond | — | cyclopropyl | 2.32* |
| 306 | CR³ | CR⁴ | H | H | (tert- | H | H | H | H | Cl | H | bond | — | cyclopropyl | 2.3** |
| | | | | | butylamino)- | | | | | | | | | | |
| | | | | | sulphonyl | | | | | | | | | | |
| 307 | CR³ | CR⁴ | H | H | pyrrolidin-1- | H | H | H | H | Cl | H | bond | — | cyclopropyl | 2.52** |
| | | | | | ylsulphonyl- | | | | | | | | | | |
| 308 | CR³ | CR⁴ | H | H | ethoxy | H | H | H | H | Br | H | bond | — | cyclopropyl | 1.87* |
| 309 | CR³ | CR⁴ | H | H | H | (methoxy- | H | H | H | Br | H | bond | — | cyclopropyl | 1.61* |
| | | | | | | carbonyl)amino | | | | | | | | | |
| 310 | CR³ | CR⁴ | H | H | 1-methylcyclo- | H | H | H | H | Cl | H | bond | — | cyclopropyl | 3.42* |
| | | | | | pentyl | | | | | | | | | | |
| 311 | CR³ | CR⁴ | H | H | Cl | difluoromethoxy | H | H | H | Br | H | bond | — | cyclopropyl | 3.44* |
| 312 | CR³ | CR⁴ | H | H | H | NHCOtBu | H | H | H | Cl | H | methylene | — | cyclopropyl | 2.14* |
| 313 | CR³ | CR⁴ | Me | H | amino | Me | H | H | H | Cl | H | bond | — | cyclopropyl | 0.87** |
| 314 | CR³ | CR⁴ | H | H | (morpholin-4- | H | H | H | H | Cl | H | bond | — | cyclopropyl | 1.57** |
| | | | | | ylsulphonyl)methyl | | | | | | | | | | |
| 315 | CR³ | CR⁴ | H | H | H | H | H | H | H | Cl | H | bond | — | 1-(ethoxy- | 2.17* |
| | | | | | | | | | | | | | | carbonyl)- | |
| | | | | | | | | | | | | | | cycloproyl | |
| 316 | CR³ | CR⁴ | H | H | H | Cl | H | H | H | Cl | H | bond | — | 1-(ethoxy- | 3.13* |
| | | | | | | | | | | | | | | carbonyl)- | |
| | | | | | | | | | | | | | | cycloproyl | |
| 317 | CR³ | CR⁴ | H | OMe | OMe | OMe | H | H | H | Cl | H | bond | — | 1-(ethoxy- | 2.01* |
| | | | | | | | | | | | | | | carbonyl)- | |
| | | | | | | | | | | | | | | cycloproyl | |
| 318 | CR³ | CR⁴ | H | H | H | H | H | H | H | Cl | H | bond | — | 1-(2- | 3.66* |
| | | | | | | | | | | | | | | chlorophen- | |
| | | | | | | | | | | | | | | yl)cyclopropyl | |
| 319 | CR³ | CR⁴ | H | H | H | Cl | H | H | H | Cl | H | bond | — | 1-(2- | 5.18* |
| | | | | | | | | | | | | | | chlorophenyl)- | |
| | | | | | | | | | | | | | | cyclopropyl | |
| 320 | CR³ | CR⁴ | H | OMe | OMe | OMe | H | H | H | Cl | H | bond | — | 1-(2- | 3.06* |
| | | | | | | | | | | | | | | chlorophenyl)- | |
| | | | | | | | | | | | | | | cyclopropyl | |
| 321 | CR³ | CR⁴ | H | H | F | F | H | H | H | Cl | H | bond | — | cyclopropyl | 2.59* |
| 322 | CR³ | CR⁴ | H | H | SMe | CF₃ | H | H | H | Cl | H | bond | — | cyclopropyl | 3.48* |
| 323 | CR³ | CR⁴ | H | H | H | 2-oxopyrrolidin- | H | H | H | Cl | H | bond | — | 1-(2- | 2.95* |
| | | | | | | 1-yl | | | | | | | | chlorophenyl)- | |
| | | | | | | | | | | | | | | cyclopropyl | |
| 324 | CR³ | CR⁴ | H | H | [(isopropylamino)- | H | H | H | H | Cl | H | bond | — | cyclopropyl | 1.75** |
| | | | | | sulphonyl]methyl | | | | | | | | | | |
| 325 | CR³ | CR⁴ | H | H | H | [(isopropylamino)- | H | H | H | Cl | H | bond | — | cyclopropyl | 1.78** |
| | | | | | | sulphonyl]methyl | | | | | | | | | |
| 326 | CR³ | CR⁴ | H | H | tert- | H | H | H | H | Cl | H | bond | — | cyclopropyl | 1.99** |
| | | | | | butylamino)- | | | | | | | | | | |
| | | | | | sulphonyl]methyl | | | | | | | | | | |
| 327 | CR³ | CR⁴ | H | H | H | [(tert- | H | H | H | Cl | H | bond | — | cyclopropyl | 2.05** |
| | | | | | | butylamino)- | | | | | | | | | |
| | | | | | | sulphonyl]methyl | | | | | | | | | |
| 328 | CR³ | CR⁴ | H | H | H | amino | H | H | H | Cl | H | bond | — | cyclopropyl | 0.74* |
| 329 | CR³ | CR⁴ | H | H | CH₂NHCOOtertBu | H | H | H | H | Br | H | bond | — | cyclopropyl | 2.02* |
| 330 | CR³ | CR⁴ | H | H | H | NHCOOtertBu | H | H | H | Cl | H | bond | — | cyclopropyl | 2.27* |
| 331 | CR³ | CR⁴ | H | H | H | (isopropoxy- | H | H | H | Br | H | bond | — | cyclopropyl | 2.11* |
| | | | | | | carbonyl)amino | | | | | | | | | |
| 332 | CR³ | CR⁴ | H | H | H | difluoromethoxy | H | H | H | Br | H | bond | — | cyclopropyl | 2.66* |
| 333 | CR³ | CR⁴ | H | H | H | 2,2,2-trifluoro- | H | H | H | Br | H | bond | — | cyclopropyl | 2.82* |
| | | | | | | 1-methoxyethyl | | | | | | | | | |
| 334 | CR³ | CR⁴ | H | H | [(cyclopropylamino) | H | H | H | H | Cl | H | bond | — | cyclopropyl | 1.58** |
| | | | | | sulphonyl]methyl | | | | | | | | | | |
| 335 | CR³ | CR⁴ | H | H | H | [(propylamino)- | H | H | H | Cl | H | bond | — | cyclopropyl | 1.81* |
| | | | | | | sulphonyl]methyl | | | | | | | | | |
| 336 | CR³ | CR⁴ | H | H | H | propoxy | H | H | H | Br | H | bond | — | cyclopropyl | 2.83* |
| 337 | CR³ | CR⁴ | N | H | acetylamino | — | H | H | H | Br | H | bond | — | cyclopropyl | 0.64* |
| 338 | CR³ | CR⁴ | H | H | (E)-methoxy- | H | H | H | H | Br | H | bond | — | cyclopropyl | 2.55* |
| | | | | | imino)-methyl | | | | | | | | | | |
| 339 | CR³ | CR⁴ | H | H | (methoxycarbonyl)- | H | H | H | H | Cl | H | bond | — | cyclopropyl | 1.63* |
| | | | | | (methyl)amino | | | | | | | | | | |
| 340 | CR³ | CR⁴ | H | H | H | ethynyl | H | H | H | Br | H | bond | — | cyclopropyl | 2.41* |
| 341 | CR³ | CR⁴ | Me | H | H | methoxycarbonyl | H | H | H | Br | H | bond | — | cyclopropyl | 2.05* |
| 342 | CR³ | CR⁴ | H | H | 2-oxopyrrolidin- | H | H | H | H | CF₃ | H | bond | — | cyclopropyl | 2.12** |
| | | | | | 1-yl | | | | | | | | | | |
| 343 | CR³ | CR⁴ | H | H | H | OMe | H | H | H | Br | H | bond | — | cyclopropyl | 1.9* |
| 344 | CR³ | CR⁴ | H | H | OMe | H | H | H | H | Br | H | bond | — | cyclopropyl | 1.63* |
| 345 | CR³ | CR⁴ | H | H | methylsulphonyl | H | H | H | H | Br | H | bond | — | cyclopropyl | 1.9* |
| 346 | CR³ | CR⁴ | H | H | SO₂NMe₂ | H | H | H | H | Br | H | bond | — | cyclopropyl | 2.33* |
| 347 | CR³ | CR⁴ | H | H | NHCOtBu | H | H | H | H | Br | H | bond | — | cyclopropyl | 1.83* |
| 348 | CR³ | CR⁴ | H | H | NHCOMe | H | H | H | H | Br | H | bond | — | cyclopropyl | 1.31* |
| 349 | CR³ | CR⁴ | H | H | acetyl(methyl)- | H | H | H | H | Br | H | bond | — | cyclopropyl | 1.54* |
| | | | | | amino | | | | | | | | | | |
| 350 | CR³ | CR⁴ | H | H | (trifluoroacetyl)- | H | H | H | H | Br | H | bond | — | cyclopropyl | 1.97* |
| | | | | | amino | | | | | | | | | | |
| 351 | CR³ | CR⁴ | H | H | dimethylcarbamoyl | H | H | H | H | Br | H | bond | — | cyclopropyl | 1.56* |
| 352 | CR³ | CR⁴ | H | H | tert- | H | H | H | H | Br | H | bond | — | cyclopropyl | 2.18* |
| | | | | | butylcarbamoyl | | | | | | | | | | |
| 353 | CR³ | CR⁴ | H | H | SMe | H | H | H | H | Br | H | bond | — | cyclopropyl | 2.18* |
| 354 | CR³ | CR⁴ | H | H | H | piperidin-1- | H | H | H | Br | H | bond | — | cyclopropyl | 2.16* |
| | | | | | | ylsulphonyl | | | | | | | | | |
| 355 | CR³ | CR⁴ | H | H | 3,3-dimethyl-2- | H | H | H | H | Br | H | bond | — | cyclopropyl | 2.39* |
| | | | | | oxobutyl | | | | | | | | | | |
| 356 | CR³ | CR⁴ | H | H | propionyl | H | H | H | H | Br | H | bond | — | cyclopropyl | 2.68* |
| 357 | CR³ | CR⁴ | H | H | COMe | H | H | H | H | Br | H | bond | — | cyclopropyl | 2.09* |
| 358 | CR³ | CR⁴ | H | H | H | COMe | H | H | H | Br | H | bond | — | cyclopropyl | 1.85* |
| 359 | CR³ | CR⁴ | H | H | H | Cl | H | H | H | Br | H | bond | — | cyclopropyl | 2.77* |
| 360 | CR³ | CR⁴ | H | H | H | 2-oxopyrrolidin- | H | H | H | Cl | H | bond | Citrat | cyclopropyl | 1.52** |
| | | | | | | 1-yl | | | | | | | | | |
| 361 | CR³ | CR⁴ | H | 2- | H | H | H | H | H | Cl | H | bond | KHSO4 | cyclopropyl | 1.52** |
| | | | | oxopyrro- | | | | | | | | | | | |
| | | | | lidin-1-yl | | | | | | | | | | | |
| 362 | CR³ | CR⁴ | H | 2- | H | H | H | H | H | Cl | H | bond | HSO3 | cyclopropyl | 1.52** |
| | | | | oxopyrro- | | | | | | | | | | | |
| | | | | lidin-1-yl | | | | | | | | | | | |
| 363 | CR³ | CR⁴ | H | H | H | isopropyl | H | H | H | Br | H | bond | — | cyclopropyl | 2.8* |
| 364 | CR³ | CR⁴ | H | H | H | H | H | H | H | I | H | bond | — | cyclopropyl | 1.82* |
| 365 | CR³ | CR⁴ | H | H | (methoxycarbono- | H | H | H | H | Cl | H | bond | — | cyclopropyl | 1.8* |
| | | | | | thioyl)amino | | | | | | | | | | |
| 366 | CR³ | CR⁴ | H | H | Me | hydroxy | H | H | H | Br | H | bond | — | cyclopropyl | 1.46* |
| 367 | CR³ | CR⁴ | H | H | 2-oxo-1,3- | H | H | H | H | CF₃ | H | bond | — | cyclopropyl | 1.94*; |
| | | | | | oxazolidin-3-yl | | | | | | | | | | 2.04** |
| 368 | CR³ | CR⁴ | H | H | Cl | 2-oxopyrrolidin- | H | H | H | Cl | H | bond | — | cyclopropyl | 1.9** |
| | | | | | | 1-yl | | | | | | | | | |
| 369 | CR³ | CR⁴ | H | H | (anilinosulphonyl)- | H | H | H | H | Cl | H | bond | — | cyclopropyl | 2.18* |
| | | | | | methyl | | | | | | | | | | |
| 370 | CR³ | CR⁴ | H | H | H | (anilinosulphonyl)- | H | H | H | Cl | H | bond | — | cyclopropyl | 2.15* |
| | | | | | | methyl | | | | | | | | | |
| 371 | CR³ | CR⁴ | H | H | [(propylamino)- | H | H | H | H | Cl | H | bond | — | cyclopropyl | 1.79* |
| | | | | | sulphonyl]methyl | | | | | | | | | | |
| 372 | CR³ | CR⁴ | H | H | H | [(cyclopropyl- | H | H | H | Cl | H | bond | — | cyclopropyl | 1.59* |
| | | | | | | amino)sulphonyl]- | | | | | | | | | |
| | | | | | | methyl | | | | | | | | | |
| 373 | CR³ | CR⁴ | H | H | H | (2-oxopiperidin- | H | H | H | Cl | H | bond | — | cyclopropyl | 1.58* |
| | | | | | | 1-yl)methyl | | | | | | | | | |
| 374 | CR³ | CR⁴ | H | H | CN | H | H | H | H | Cl | H | bond | — | cyclopropyl | 2.33* |
| 375 | CR³ | CR⁴ | H | H | trifluoromethoxy | H | H | H | H | Cl | H | bond | — | cyclopropyl | 2.84* |
| 376 | CR³ | CR⁴ | H | H | (trifluoromethyl)thio | H | H | H | H | Cl | H | bond | — | cyclopropyl | 3.72* |
| 377 | CR³ | CR⁴ | H | H | SMe | Cl | H | H | H | Cl | H | bond | — | cyclopropyl | 2.84* |
| 378 | CR³ | CR⁴ | H | H | methoxycarbonyl | Cl | H | H | H | Cl | H | bond | — | cyclopropyl | 2.91* |
| 379 | CR³ | CR⁴ | H | H | NHCOMe | Cl | H | H | H | Br | H | bond | — | cyclopropyl | 1.64* |
| 380 | CR³ | CR⁴ | H | H | acetyl(ethyl)amino | H | H | H | H | Br | H | bond | — | cyclopropyl | 1.73* |
| 381 | CR³ | CR⁴ | H | H | H | propionylamino | H | H | H | Br | H | bond | — | cyclopropyl | 1.54* |
| 382 | CR³ | CR⁴ | H | H | methylcarbamoyl | H | H | H | H | Br | H | bond | — | cyclopropyl | 1.39* |
| 383 | CR³ | CR⁴ | H | H | H | methylcarbamoyl | H | H | H | Br | H | bond | — | cyclopropyl | 1.39* |
| 384 | CR³ | CR⁴ | H | H | 2,5- | H | H | H | H | Br | H | bond | — | cyclopropyl | 0.62* |
| | | | | | dioxoimidazo- | | | | | | | | | | |
| | | | | | lidin-4-yl | | | | | | | | | | |
| 385 | CR³ | CR⁴ | H | H | H | (ethoxycarbonyl)- | H | H | H | Br | H | bond | — | cyclopropyl | 1.83* |
| | | | | | | amino | | | | | | | | | |
| 386 | CR³ | CR⁴ | H | H | H | (methylcarbamoyl)- | H | H | H | Br | H | bond | — | cyclopropyl | 1.56* |
| | | | | | | oxy | | | | | | | | | |
| 387 | CR³ | CR⁴ | H | H | —NHCOCH₂— | | H | H | H | Br | H | bond | | cyclopropyl | 1.16* |
| 388 | CR³ | CR⁴ | H | —CH₂CH₂N(COCH₃)— | | H | H | H | H | Br | H | bond | | cyclopropyl | 1.54* |
| 389 | CR³ | CR⁴ | H | H | H | dimethylcarbamoyl | H | H | H | Br | H | bond | — | cyclopropyl | 1.42* |
| 390 | CR³ | CR⁴ | H | H | —CH₂CH₂N(COCH₃)— | | H | H | H | Br | H | bond | | cyclopropyl | 1.61* |
| 391 | CR³ | CR⁴ | H | H | NHCOOtBu | H | H | H | H | Br | H | bond | — | cyclopropyl | 2.13* |
| 392 | CR³ | CR⁴ | H | H | 3-methyl-2,5- | H | H | H | H | Br | H | bond | — | cyclopropyl | 1.46* |
| | | | | | dioxoimidazolidin- | | | | | | | | | | |
| | | | | | 1-yl | | | | | | | | | | |
| 393 | CR³ | CR⁴ | H | H | H | NHCOOtBu | H | H | H | Br | H | bond | — | cyclopropyl | 2.29* |
| 394 | CR³ | CR⁴ | H | H | NHCOisoPr | H | H | H | H | Br | H | bond | — | cyclopropyl | 1.64* |
| 395 | CR³ | CR⁴ | H | H | (2-oxopiperidin- | H | H | H | H | Cl | H | bond | — | cyclopropyl | 1.5** |
| | | | | | 1-yl)methyl | | | | | | | | | | |
| 396 | CR³ | CR⁴ | H | H | H | 2-oxopyrrolidin- | H | H | H | Cl | H | bond | — | 1- | 1.83* |
| | | | | | | 1-yl | | | | | | | | (ethoxycarbon- | |
| | | | | | | | | | | | | | | yl)cyclopropyl | |
| 397 | CR³ | CR⁴ | H | H | H | pyrrolidin-1- | H | H | H | Br | H | bond | — | cyclopropyl | 0.92* |
| | | | | | | ylmethyl | | | | | | | | | |
| 398 | CR³ | CR⁴ | H | H | isobutoxy | H | H | H | H | Br | H | bond | — | cyclopropyl | 2.73* |
| 399 | CR³ | CR⁴ | H | H | OMe | Cl | H | H | H | Br | H | bond | — | cyclopropyl | 2.25* |
| 400 | CR³ | CR⁴ | H | H | 2-methoxyethoxy | Cl | H | H | H | Br | H | bond | — | cyclopropyl | 0.23* |
| 401 | CR³ | CR⁴ | H | H | H | (methoxyacetyl)- | H | H | H | Br | H | bond | — | cyclopropyl | 1.46* |
| | | | | | | amino | | | | | | | | | |
| 402 | CR³ | CR⁴ | H | H | H | allyloxy | H | H | H | Br | H | bond | — | cyclopropyl | 2.55* |
| 403 | CR³ | CR⁴ | F | H | (ethoxycarbonyl)- | F | H | H | H | Cl | H | bond | — | cyclopropyl | 2.69* |
| | | | | | amino | | | | | | | | | | |
| 404 | CR³ | CR⁴ | H | H | H | (1E)-N-methoxy- | H | H | H | Br | H | bond | — | cyclopropyl | 2.57* |
| | | | | | | ethaneimidoyl | | | | | | | | | |
| 405 | CR³ | CR⁴ | H | F | H | (methoxycarbonyl)- | H | H | H | Br | H | bond | — | cyclopropyl | 1.83* |
| | | | | | | amino | | | | | | | | | |
| 406 | CR³ | CR⁴ | H | H | F | H | H | H | H | Br | H | methylene | — | cyclopropyl | 2.3* |
| 407 | CR³ | CR⁴ | H | H | CF₃ | H | H | H | H | Br | H | methylene | — | cyclopropyl | 3.55* |
| 408 | CR³ | CR⁴ | H | H | H | CN | H | H | H | Br | H | methylene | — | cyclopropyl | 2.4* |
| 409 | CR³ | CR⁴ | H | H | OMe | OMe | H | H | H | Br | H | methylene | — | cyclopropyl | 1.68* |
| 410 | CR³ | CR⁴ | H | H | H | OMe | H | H | H | Br | H | methylene | — | cyclopropyl | 2.06* |
| 411 | CR³ | CR⁴ | H | H | OMe | H | H | H | H | Br | H | methylene | — | cyclopropyl | 1.83* |
| 412 | CR³ | CR⁴ | H | H | SMe | Cl | H | H | H | Br | H | methylene | — | cyclopropyl | 3.1* |
| 413 | CR³ | CR⁴ | H | H | 1,3-thiazol-4-yl | H | H | H | H | Br | H | methylene | — | cyclopropyl | 2.16* |
| 414 | CR³ | CR⁴ | H | H | H | 2-methoxy-2- | H | H | H | Br | H | methylene | — | cyclopropyl | 2.02* |
| | | | | | | oxoethyl | | | | | | | | | |
| 415 | CR³ | CR⁴ | H | Cl | H | Cl | H | H | H | Br | H | methylene | — | cyclopropyl | 4.51* |
| 416 | CR³ | CR⁴ | H | H | methoxycarbonyl | Cl | H | H | H | Br | H | methylene | — | cyclopropyl | 3.29* |
| 417 | CR³ | CR⁴ | H | H | H | ethoxy | H | H | H | Br | H | methylene | — | cyclopropyl | 2.33* |
| 418 | CR³ | CR⁴ | H | H | methylenebis(oxy) | | H | H | H | Br | H | methylene | — | cyclopropyl | 1.83* |
| 419 | CR³ | CR⁴ | H | H | acetyl(methyl)amino | H | H | H | H | Br | H | methylene | — | cyclopropyl | 1.64* |
| 420 | CR³ | CR⁴ | H | H | Cl | methylsulphonyl | H | H | H | Br | H | methylene | — | cyclopropyl | 2.36* |
| 421 | CR³ | CR⁴ | H | —CH₂CH₂N(COCH₃)— | | H | H | H | H | Br | H | methylene | — | cyclopropyl | 1.66* |
| 422 | CR³ | CR⁴ | H | H | NHCOMe | OMe | H | H | H | Br | H | methylene | — | cyclopropyl | 1.59* |
| 423 | CR³ | CR⁴ | H | H | Cl | SO₂NH₂ | H | H | H | Br | H | methylene | — | cyclopropyl | 1.92* |
| 424 | CR³ | CR⁴ | H | H | methylcarbamoyl | H | H | H | H | Br | H | methylene | — | cyclopropyl | 1.58* |
| 425 | CR³ | CR⁴ | H | H | allylcarbamoyl | H | H | H | H | Br | H | methylene | — | cyclopropyl | 1.9* |
| 426 | CR³ | CR⁴ | H | H | tert- | H | H | H | H | Br | H | methylene | — | cyclopropyl | 2.3* |
| | | | | | butylcarbamoyl | | | | | | | | | | |
| 427 | CR³ | CR⁴ | H | H | H | methylcarbomoyl | H | H | H | Br | H | methylene | — | cyclopropyl | 1.53* |
| 428 | CR³ | CR⁴ | H | H | H | (ethoxycarbonyl)- | H | H | H | Br | H | methylene | — | cyclopropyl | 1.99* |
| | | | | | | amino | | | | | | | | | |
| 429 | CR³ | CR⁴ | H | H | H | (methylcarbamoyl)- | H | H | H | Br | H | methylene | — | cyclopropyl | 1.68* |
| | | | | | | oxy | | | | | | | | | |
| 430 | CR³ | CR⁴ | H | H | (methoxycarbonyl)- | Cl | H | H | H | Br | H | bond | — | cyclopropyl | 2.2* |
| | | | | | amino | | | | | | | | | | |
| 431 | CR³ | CR⁴ | H | H | H | [(dimethylamino)- | H | H | H | Cl | H | bond | — | cyclopropyl | 1.62* |
| | | | | | | sulphonyl]methyl | | | | | | | | | |
| 432 | CR³ | CR⁴ | H | H | (2-oxopyrrolidin- | H | H | H | H | Cl | H | bond | — | cyclopropyl | 1.36* |
| | | | | | 1-yl)methyl | | | | | | | | | | |
| 433 | CR³ | CR⁴ | H | H | H | (2-oxopyrrolidin-1- | H | H | H | Cl | H | bond | — | cyclopropyl | 1.45* |
| | | | | | | yl)methyl | | | | | | | | | |
| 434 | CR³ | CR⁴ | H | H | NHCOMe | H | H | H | H | Cl | H | bond | TsOH | cyclopropyl | 1.02* |
| 435 | CR³ | CR⁴ | H | H | H | isobutylthio | H | H | H | Cl | H | bond | — | cyclopropyl | 3.75* |
| 436 | CR³ | CR⁴ | H | H | H | ethylthio | H | H | H | Cl | H | bond | — | cyclopropyl | 2.72* |
| 437 | CR³ | CR⁴ | H | H | H | propylthio | H | H | H | Cl | H | bond | — | cyclopropyl | 3.33* |
| 438 | CR³ | CR⁴ | H | H | nitro | H | H | H | H | Cl | H | bond | — | cyclopropyl | 2.9** |
| 439 | CR³ | CR⁴ | H | H | pyrrolidin-1-yl | CN | H | H | H | Cl | H | bond | — | cyclopropyl | 2.23** |
| 440 | CR³ | CR⁴ | H | ethylthio | H | H | H | H | H | Cl | H | bond | TsOH | cyclopropyl | 2.73* |
| 441 | CR³ | CR⁴ | H | (3-methyl- | H | H | H | H | H | Cl | H | bond | TsOH | cyclopropyl | 4.24* |
| | | | | butyl)thio | | | | | | | | | | | |
| 442 | CR³ | CR⁴ | H | octylthio | H | H | H | H | H | Cl | H | bond | TsOH | cyclopropyl | 6.16* |
| 443 | CR³ | CR⁴ | H | butylthio | H | H | H | H | H | Cl | H | bond | TsOH | cyclopropyl | 3.79* |
| 444 | CR³ | CR⁴ | H | H | H | isobutoxy | H | H | H | Cl | H | bond | — | cyclopropyl | 3.17* |
| 445 | CR³ | CR⁴ | H | H | H | isobutoxy | H | H | H | Br | H | bond | — | cyclopropyl | 3.36* |
| 446 | CR³ | CR⁴ | H | H | (methoxy- | H | H | H | H | Me | H | bond | — | cyclopropyl | 1.06* |
| | | | | | carbonyl)amino | | | | | | | | | | |
| 447 | CR³ | CR⁴ | H | H | H | 2,5-dioxopyrrolidin- | H | H | H | Br | H | bond | — | cyclopropyl | 1.38* |
| | | | | | | 1-yl | | | | | | | | | |
| 448 | CR³ | CR⁴ | H | H | methylsulphonyl | H | H | H | H | Br | H | methylene | — | cyclopropyl | 2.09* |
| 449 | CR³ | CR⁴ | H | H | SO₂NMe₂ | H | H | H | H | Br | H | methylene | — | cyclopropyl | 2.51* |
| 450 | CR³ | CR⁴ | H | H | NHCOtBu | H | H | H | H | Br | H | methylene | — | cyclopropyl | 1.99* |
| 451 | CR³ | CR⁴ | H | H | H | NHCOtertBu | H | H | H | Br | H | methylene | — | cyclopropyl | 2.06* |
| 452 | CR³ | CR⁴ | H | H | dimethylcarbamoyl | H | H | H | H | Br | H | methylene | — | cyclopropyl | 1.66*; |
| 453 | CR³ | CR⁴ | H | H | SMe | H | H | H | H | Br | H | methylene | — | cyclopropyl | 2.29* |
| 454 | CR³ | CR⁴ | H | H | H | piperidin-1- | H | H | H | Br | H | methylene | — | cyclopropyl | 2.29* |
| | | | | | | ylsulphonyl | | | | | | | | | |
| 455 | CR³ | CR⁴ | H | H | 3,3-dimethyl-2- | H | H | H | H | Br | H | methylene | — | cyclopropyl | 2.48* |
| | | | | | oxobutyl | | | | | | | | | | |
| 456 | CR³ | CR⁴ | H | H | COMe | H | H | H | H | Br | H | methylene | — | cyclopropyl | 2.26* |
| 457 | CR³ | CR⁴ | H | H | 2-oxopyrrolidin- | H | H | H | H | Br | H | methylene | — | cyclopropyl | 1.66*; |
| | | | | | 1-yl | | | | | | | methylene | | | 1.59** |
| 458 | CR³ | CR⁴ | H | H | H | (methoxycarbonyl)- | H | H | H | Br | H | methylene | — | cyclopropyl | 1.73* |
| | | | | | | amino | | | | | | | | | |
| 459 | CR³ | CR⁴ | H | H | Me | SMe | H | H | H | Br | H | methylene | — | cyclopropyl | 2.58* |
| 460 | CR³ | CR⁴ | H | H | H | NHCOisoPr | H | H | H | Br | H | methylene | — | cyclopropyl | 1.85* |
| 461 | CR³ | CR⁴ | H | H | H | tert- | H | H | H | Br | H | methylene | — | cyclopropyl | 2.16* |
| | | | | | | butylcarbamoyl | | | | | | | | | |
| 462 | CR³ | CR⁴ | H | H | 1,1,2,2- | H | H | H | H | Br | H | methylene | — | cyclopropyl | 2.8* |
| | | | | | tetrafluoroethoxy | | | | | | | | | | |
| 463 | CR³ | CR⁴ | H | H | hydroxy | COMe | H | H | H | Br | H | methylene | — | cyclopropyl | 1.92* |
| 464 | CR³ | CR⁴ | H | H | hydroxy | F | H | H | H | Br | H | methylene | — | cyclopropyl | 1.53* |
| 465 | CR³ | CR⁴ | H | H | cyclopropyl- | H | H | H | H | Br | H | methylene | — | cyclopropyl | 2.74* |
| | | | | | trifluoroacetyl)- | | | | | | | | | | |
| | | | | | amino | | | | | | | | | | |
| 466 | CR³ | CR⁴ | H | H | H | (phenoxycarbonyl)- | H | H | H | Br | H | methylene | — | cyclopropyl | 2.36* |
| | | | | | | amino | | | | | | | | | |
| 467 | CR³ | CR⁴ | H | H | H | (3-fluoro-2,2- | H | H | H | Br | H | methylene | — | cyclopropyl | 1.99* |
| | | | | | | dimethylpropanoyl)- | | | | | | | | | |
| | | | | | | amino | | | | | | | | | |
| 468 | CR³ | CR⁴ | H | H | Me | NHCOMe | H | H | H | Br | H | methylene | — | cyclopropyl | 1.51* |
| 469 | CR³ | CR⁴ | H | H | NHCOisoPr | H | H | H | H | Br | H | methylene | — | cyclopropyl | 1.78* |
| 470 | CR³ | CR⁴ | H | H | (difluoromethyl)- | H | H | H | H | Br | H | methylene | — | cyclopropyl | 3.07* |
| | | | | | thio | | | | | | | | | | |
| 471 | CR³ | CR⁴ | H | Me | H | Me | H | H | H | Br | H | methylene | — | cyclopropyl | 2.36* |
| 472 | CR³ | CR⁴ | H | H | Br | H | H | H | H | Br | H | methylene | — | cyclopropyl | 2.84* |
| 473 | CR³ | CR⁴ | H | H | H | H | OMe | H | H | Br | H | methylene | — | cyclopropyl | 2.09* |
| 474 | CR³ | CR⁴ | H | H | Cl | H | H | H | H | Br | H | methylene | — | cyclopropyl | 2.67* |
| 475 | CR³ | CR⁴ | H | H | CN | H | H | H | H | Br | H | methylene | — | cyclopropyl | 2.7* |
| 476 | CR³ | CR⁴ | H | H | trifluoromethoxy | H | H | H | H | Br | H | methylene | — | cyclopropyl | 3.1* |
| 477 | CR³ | CR⁴ | H | H | H | ethoxycarbonyl | H | H | H | Br | H | methylene | — | cyclopropyl | 2.51* |
| 478 | CR³ | CR⁴ | H | H | hydroxyl | H | F | H | H | Br | H | methylene | — | cyclopropyl | 1.51* |
| 479 | CR³ | CR⁴ | H | H | NHCOMe | Cl | H | H | H | Br | H | methylene | — | cyclopropyl | 1.68* |
| 480 | CR³ | CR⁴ | H | H | acetyl(ethyl)amino | H | H | H | H | Br | H | methylene | — | cyclopropyl | 1.83* |
| 481 | CR³ | N | H | H | acetylamino | — | H | H | H | Br | H | methylene | — | cyclopropyl | 1.47* |
| 482 | CR³ | CR⁴ | H | H | H | acryloylamino | H | H | H | Br | H | methylene | — | cyclopropyl | 1.66* |
| 483 | CR³ | CR⁴ | H | H | H | propionylamino | H | H | H | Br | H | methylene | — | cyclopropyl | 1.66* |
| 484 | CR³ | CR⁴ | H | H | morpholin-4- | H | H | H | H | Br | H | methylene | — | cyclopropyl | 1.64* |
| | | | | | ylcarbonyl | | | | | | | | | | |
| 485 | CR³ | CR⁴ | H | H | H | [(dimethylamino)- | H | H | H | Br | H | methylene | — | cyclopropyl | 2.51* |
| | | | | | | sulphonyl]oxy | | | | | | | | | |
| 486 | CR³ | CR⁴ | H | H | 2-ethoxy-2-oxoethyl | H | H | H | H | Br | H | methylene | — | cyclopropyl | 2.18* |
| 487 | CR³ | CR⁴ | H | H | Cl | COMe | H | H | H | Br | H | methylene | — | cyclopropyl | 2.67* |
| 488 | CR³ | CR⁴ | H | H | hydroxyl | propionyl | H | H | H | Br | H | methylene | — | cyclopropyl | 2.21* |
| 489 | CR³ | CR⁴ | H | H | (ethoxycarbonyl)- | hydroxyl | H | H | H | Br | H | methylene | — | cyclopropyl | 1.75* |
| | | | | | amoino | | | | | | | | | | |
| 490 | CR³ | CR⁴ | H | H | formyl(methyl)- | H | H | H | H | Br | H | methylene | — | cyclopropyl | 1.63* |
| | | | | | amoino | | | | | | | | | | |
| 491 | CR³ | CR⁴ | H | H | hydroxyl | H | Me | H | H | Br | H | methylene | — | cyclopropyl | 1.47* |
| 492 | CR³ | CR⁴ | H | H | H | benzoylamino | H | H | H | Br | H | methylene | — | cyclopropyl | 2.29* |
| 493 | CR³ | CR⁴ | H | H | H | 1,1,2,2- | H | H | H | Br | H | methylene | — | cyclopropyl | 3.1* |
| | | | | | | tetrafluoroethoxy | | | | | | | | | |
| 494 | CR³ | CR⁴ | H | H | hydroxyl | propionylamino | H | H | H | Br | H | methylene | — | cyclopropyl | 1.97* |
| 495 | CR³ | CR⁴ | H | H | 3-oxomorpholin- | H | H | H | H | Cl | H | bond | — | cyclopropyl | 1.17* |
| | | | | | 4-yl | | | | | | | | | | |
| 496 | CR³ | CR⁴ | F | H | (ethoxycarbonyl)- | F | H | H | H | Br | H | bond | — | cyclopropyl | 2.84* |
| | | | | | amino | | | | | | | | | | |
| 497 | CR³ | CR⁴ | H | H | H | SMe | H | H | H | Me | H | bond | — | cyclopropyl | 1.48* |
| 498 | CR³ | CR⁴ | H | H | F | H | H | H | H | CF₃ | H | bond | — | cyclopropyl | 2.96** |
| 499 | CR³ | CR⁴ | H | H | F | H | H | H | H | Br | H | bond | — | cyclopropyl | 1.96* |
| 500 | CR³ | CR⁴ | H | H | 3-oxomorpholin- | H | H | H | H | Br | H | bond | — | cyclopropyl | 1.29* |
| | | | | | 4-yl | | | | | | | | | | |
| 501 | CR³ | CR⁴ | H | H | 1H-imidazol-1-yl | H | H | H | H | Br | H | bond | — | cyclopropyl | 0.93* |
| 502 | CR³ | CR⁴ | H | H | H | ethynyl | H | H | H | Cl | H | bond | — | cyclopropyl | 2.24* |
| 503 | CR³ | CR⁴ | H | H | NHCOMe | OMe | H | H | H | Cl | H | methylene | — | cyclopropyl | 1.53* |
| 504 | CR³ | CR⁴ | H | H | H | 2-methoxy-2- | H | H | H | Cl | H | methylene | — | cyclopropyl | 1.92* |
| | | | | | | oxoethyl | | | | | | | | | |
| 505 | CR³ | CR⁴ | H | H | NHCOMe | Cl | H | H | H | Cl | H | methylene | — | cyclopropyl | 1.68* |
| 506 | CR³ | CR⁴ | H | H | acetyl(ethyl)amino | H | H | H | H | Cl | H | methylene | — | cyclopropyl | 1.73* |
| 507 | CR³ | N | H | H | acetylamino | — | H | H | H | Cl | H | methylene | — | cyclopropyl | 0.89* |
| 508 | CR³ | CR⁴ | H | H | H | propionylamino | H | H | H | Cl | H | methylene | — | cyclopropyl | 1.61* |
| 509 | CR³ | CR⁴ | H | H | dimethylcarbamoyl | H | H | H | H | Cl | H | methylene | — | cyclopropyl | 1.59* |
| 510 | CR³ | CR⁴ | H | H | methylcarbamoyl | H | H | H | H | Cl | H | methylene | — | cyclopropyl | 1.44* |
| 511 | CR³ | CR⁴ | H | H | H | methylcarbamoyl | H | H | H | Cl | H | methylene | — | cyclopropyl | 1.44* |
| 512 | CR³ | CR⁴ | H | H | 2,5-dioxoimidazol- | H | H | H | H | Cl | H | methylene | — | cyclopropyl | 1.27* |
| | | | | | idin-4-yl | | | | | | | | | | |
| 513 | CR³ | CR⁴ | H | H | H | (ethoxycarbonyl)- | H | H | H | Cl | H | methylene | — | cyclopropyl | 1.9* |
| | | | | | | amino | | | | | | | | | |
| 514 | CR³ | CR⁴ | H | H | H | (methylcarbamoyl)- | H | H | H | Cl | H | methylene | — | cyclopropyl | 1.61* |
| | | | | | | oxy | | | | | | | | | |
| 515 | CR³ | CR⁴ | H | H | H | (methoxycarbonyl)- | H | H | H | Cl | H | methylene | — | cyclopropyl | 1.66* |
| | | | | | | amino | | | | | | | | | |
| 516 | CR³ | CR⁴ | H | H | —NHCOCH₂— | | H | H | H | Cl | H | methylene | | cyclopropyl | 1.31* |
| 517 | CR³ | CR⁴ | H | H | —NHCOCH₂CH₂— | | H | H | H | Cl | H | methylene | | cyclopropyl | 1.19* |
| 518 | CR³ | CR⁴ | H | —CH₂CH₂N(COCH₃)— | | H | H | H | H | Cl | H | methylene | | cyclopropyl | 1.59* |
| 519 | CR³ | CR⁴ | H | H | H | dimethylcarbamoyl | H | H | H | Cl | H | methylene | — | cyclopropyl | 1.58* |
| 520 | CR³ | CR⁴ | H | H | H | 2-oxopyrrolidin- | H | H | H | Cl | H | methylene | — | cyclopropyl | 1.56* |
| | | | | | | 1-yl | | | | | | | | | |
| 521 | CR³ | CR⁴ | H | H | —CH₂CH₂N(COCH₃)— | | H | H | H | Cl | H | methylene | — | cyclopropyl | 1.66* |
| 522 | CR³ | CR⁴ | H | H | NHCOOtBu | H | H | H | H | Cl | H | methylene | — | cyclopropyl | 2.18* |
| 523 | CR³ | CR⁴ | H | H | 3-methyl-2,5- | H | H | H | H | Cl | H | methylene | — | cyclopropyl | 1.53* |
| | | | | | dioxoimidazolidin- | | | | | | | | | | |
| | | | | | 1-yl | | | | | | | | | | |
| 524 | CR³ | CR⁴ | H | H | H | NHCOOtBu | H | H | H | Cl | H | methylene | — | cyclopropyl | 2.33* |
| 525 | CR³ | CR⁴ | H | H | 2,2-dimethylprop- | H | H | H | H | Cl | H | methylene | — | cyclopropyl | 2.02* |
| | | | | | anoyl)(methyl)amino | | | | | | | | | | |
| 526 | CR³ | CR⁴ | H | H | NHCOisoPr | H | H | H | H | Cl | H | methylene | — | cyclopropyl | 1.73* |
| 527 | CR³ | CR⁴ | H | H | H | 1,1,2,2-tetra- | H | H | H | Cl | H | methylene | — | cyclopropyl | 2.95* |
| | | | | | | fluoroethoxy | | | | | | | | | |
| 528 | CR³ | CR⁴ | H | H | H | 3,5-dimethyl- | H | H | H | Cl | H | bond | — | cyclopropyl | 2.46* |
| | | | | | | piperidin-1-yl | | | | | | | | | |
| 529 | CR³ | CR⁴ | H | H | H | 3,5-dimethyl- | H | H | H | Br | H | bond | — | cyclopropyl | 2.63* |
| | | | | | | piperidin-1-yl | | | | | | | | | |
| 530 | CR³ | CR⁴ | H | H | (methoxycarbonyl)- | H | F | H | H | Cl | H | bond | — | cyclopropyl | 1.56* |
| | | | | | amino | | | | | | | | | | |
| 531 | CR³ | CR⁴ | H | H | H | propoxy | H | H | H | Cl | H | bond | — | cyclopropyl | 2.64* |
| 532 | CR³ | CR⁴ | H | H | Cl | OMe | H | H | H | Br | H | bond | — | cyclopropyl | 2.70* |
| 533 | CR³ | CR⁴ | H | H | H | 4-methyl-2- | H | H | H | Br | H | bond | — | cyclopropyl | 1.68* |
| | | | | | | oxo-1,3-oxazolidin- | | | | | | | | | |
| | | | | | | 3-yl | | | | | | | | | |
| 534 | CR³ | CR⁴ | H | H | H | difluoromethoxy | H | H | H | Cl | H | bond | — | cyclopropyl | 2.51* |
| 535 | CR³ | CR⁴ | H | H | H | piperidin-1-yl | H | H | H | Cl | H | bond | — | cyclopropyl | 1.45* |
| 536 | CR³ | CR⁴ | H | H | F | 2-oxopyrrolidin- | H | H | H | Cl | H | bond | — | cyclopropyl | 1.53* |
| | | | | | | 1-yl | | | | | | | | | |
| 537 | CR³ | CR⁴ | H | H | (piperidin-1- | H | H | H | H | Cl | H | bond | — | cyclopropyl | 2.17* |
| | | | | | ylsulphonyl)methyl | | | | | | | | | | |
| 538 | CR³ | CR⁴ | H | H | difluoromethoxy | H | H | H | H | Cl | H | methylene | — | cyclopropyl | 2.25* |
| 539 | CR³ | CR⁴ | H | H | 2-ethoxy-2-oxoethyl | H | H | H | H | Cl | H | methylene | — | cyclopropyl | 2.09* |
| 540 | CR³ | CR⁴ | H | H | Cl | COMe | H | H | H | Cl | H | methylene | — | cyclopropyl | 2.51* |
| 541 | CR³ | CR⁴ | H | H | hydroxyl | propionyl | H | H | H | Cl | H | methylene | — | cyclopropyl | 2.13* |
| 542 | CR³ | CR⁴ | H | H | (ethoxycarbonyl)- | hydroxyl | H | H | H | Cl | H | methylene | — | cyclopropyl | 1.68* |
| | | | | | amino | | | | | | | | | | |
| 543 | CR³ | CR⁴ | H | H | (ethoxycarbonyl)- | H | H | H | H | Cl | H | methylene | — | cyclopropyl | 1.8* |
| | | | | | amino | | | | | | | | | | |
| 544 | CR³ | CR⁴ | H | H | formamido | H | H | H | H | Cl | H | methylene | — | cyclopropyl | 1.17* |
| 545 | CR³ | CR⁴ | H | H | formyl(methyl)- | H | H | H | H | Cl | H | methylene | — | cyclopropyl | 1.56* |
| | | | | | amino | | | | | | | | | | |
| 546 | CR³ | CR⁴ | H | H | H | formamido | H | H | H | Cl | H | methylene | — | cyclopropyl | 1.46* |
| 547 | CR³ | CR⁴ | H | H | Me | SMe | H | H | H | Cl | H | methylene | — | cyclopropyl | 2.45* |
| 548 | CR³ | CR⁴ | H | H | (dimethylcarbamoyl)- | H | H | H | H | Cl | H | methylene | — | cyclopropyl | 1.49* |
| | | | | | amino | | | | | | | | | | |
| 549 | CR³ | CR⁴ | H | H | H | H | H | formyl | H | Cl | H | methylene | — | cyclopropyl | 1.87* |
| 550 | CR³ | CR⁴ | H | H | F | H | H | H | H | Cl | H | methylene | — | cyclopropyl | 1.97* |
| 551 | CR³ | CR⁴ | H | H | pentanoyloxy | H | H | H | H | Cl | H | methylene | — | cyclopropyl | 3.8* |
| 552 | CR³ | CR⁴ | H | H | hydroxyl | NHCOMe | H | H | H | Cl | H | methylene | — | cyclopropyl | 1.29* |
| 553 | CR³ | CR⁴ | H | H | hydroxyl | COMe | H | H | H | Cl | H | methylene | — | cyclopropyl | 1.85* |
| 554 | CR³ | CR⁴ | H | H | hydroxyl | F | H | H | H | Cl | H | methylene | — | cyclopropyl | 1.49* |
| 555 | CR³ | CR⁴ | H | H | hydroxyl | H | Me | H | H | Cl | H | methylene | — | cyclopropyl | 1.42* |
| 556 | CR³ | CR⁴ | H | H | (3-chloro-2,2- | H | H | H | H | Cl | H | methylene | — | cyclopropyl | 2.02* |
| | | | | | (dimethylpropanoyl)- | | | | | | | | | | |
| | | | | | amino | | | | | | | | | | |
| 557 | CR³ | CR⁴ | H | H | H | benzoyl-amino | H | H | H | Cl | H | methylene | — | cyclopropyl | 2.21* |
| 558 | CR³ | CR⁴ | H | H | hydroxyl | Me | H | H | H | Cl | H | methylene | — | cyclopropyl | 1.51* |
| 559 | CR³ | CR⁴ | H | H | (difluoromethyl)- | H | H | H | H | Cl | H | methylene | — | cyclopropyl | 2.91* |
| | | | | | thio | | | | | | | | | | |
| 560 | CR³ | CR⁴ | H | H | H | hydroxy-methyl | H | H | H | Cl | H | methylene | — | cyclopropyl | 1.47* |
| 561 | CR³ | CR⁴ | H | H | H | 2-(tert-butylamino)- | H | H | H | Cl | H | methylene | — | cyclopropyl | 1.93* |
| | | | | | | 2-oxoethyl | | | | | | | | | |
| 562 | CR³ | CR⁴ | H | H | H | 2-(tert-butylamino)- | H | H | H | Cl | H | bond | — | cyclopropyl | 1.81* |
| | | | | | | 2-oxoethyl | | | | | | | | | |
| 563 | CR³ | CR⁴ | H | OMe | H | OMe | H | H | H | Br | H | bond | — | cyclopropyl | 2.15* |
| 564 | CR³ | CR⁴ | H | H | H | pyrrolidin-1-yl | H | H | H | CF₃ | H | bond | — | cyclopropyl | 3.55* |
| 565 | CR³ | CR⁴ | H | H | 2-oxopropyl | H | H | H | H | Br | H | bond | — | cyclopropyl | 1.58* |
| 566 | CR³ | CR⁴ | H | H | H | (methylsulphonyl)- | H | H | H | Cl | H | methylene | — | cyclopropyl | 1.38* |
| | | | | | | methyl | | | | | | | | | |
| 567 | CR³ | CR⁴ | H | H | H | 2-oxopyrrolidin-1-yl | H | H | H | Me | H | bond | — | cyclopropyl | 1.14* |
| 568 | CR³ | CR⁴ | H | H | F | OMe | H | H | H | Br | H | bond | — | cyclopropyl | 2.05* |
| 569 | CR³ | CR⁴ | H | H | 2-oxopiperidin-1-yl | H | H | H | H | Cl | H | bond | — | cyclopropyl | 1.47* |
| 570 | CR³ | CR⁴ | H | H | H | 3-methyl-2- | H | H | H | Br | H | bond | — | cyclopropyl | 1.58* |
| | | | | | | oxoimidazolidin- | | | | | | | | | |
| | | | | | | 1-yl | | | | | | | | | |
| 571 | CR³ | CR⁴ | H | H | H | CN | H | H | H | F | H | bond | — | cyclopropyl | 1.61* |
| 572 | CR³ | CR⁴ | H | H | NHCOMe | OMe | H | H | H | F | H | bond | — | cyclopropyl | 1.17* |
| 573 | CR³ | CR⁴ | H | H | H | OMe | H | H | H | F | H | bond | — | cyclopropyl | 1.51* |
| 574 | CR³ | CR⁴ | H | H | OMe | H | H | H | H | F | H | bond | — | cyclopropyl | 1.4* |
| 575 | CR³ | CR⁴ | H | H | CN | H | H | H | H | F | H | bond | — | cyclopropyl | 1.73* |
| 576 | CR³ | CR⁴ | H | H | H | trifluoromethyloxy | H | H | H | F | H | bond | — | cyclopropyl | 2.33* |
| 577 | CR³ | CR⁴ | H | H | methylsulphonyl | H | H | H | H | F | H | bond | — | cyclopropyl | 1.4* |
| 578 | CR³ | CR⁴ | H | H | SO₂NMe₂ | H | H | H | H | F | H | bond | — | cyclopropyl | 1.68* |
| 579 | CR³ | CR⁴ | H | H | SMe | Cl | H | H | H | F | H | bond | — | cyclopropyl | 2.13* |
| 580 | CR³ | CR⁴ | H | H | NHCOMe | Cl | H | H | H | F | H | bond | — | cyclopropyl | 1.32* |
| 581 | CR³ | CR⁴ | H | H | acetyl(ethyl)amino | H | H | H | H | F | H | bond | — | cyclopropyl | 1.38* |
| 582 | CR³ | CR⁴ | H | H | acetyl(methyl)amino | H | H | H | H | F | H | bond | — | cyclopropyl | 1.23* |
| 583 | CR³ | CR⁴ | H | H | (trifluoroacetyl)- | H | H | H | H | F | H | bond | — | cyclopropyl | 1.58* |
| | | | | | amino | | | | | | | | | | |
| 584 | CR³ | CR⁴ | H | H | methylcarbamoyl | H | H | H | H | F | H | bond | — | cyclopropyl | 1.05* |
| 585 | CR³ | CR⁴ | H | H | H | methylcarbamoyl | H | H | H | F | H | bond | — | cyclopropyl | 1.05* |
| 586 | CR³ | CR⁴ | H | H | H | SMe | H | H | H | F | H | bond | — | cyclopropyl | 1.73* |
| 587 | CR³ | CR⁴ | H | H | SMe | H | H | H | H | F | H | bond | — | cyclopropyl | 1.68* |
| 588 | CR³ | CR⁴ | H | H | H | piperidin-1- | H | H | H | F | H | bond | — | cyclopropyl | 1.61* |
| | | | | | | ylsulphonyl | | | | | | | | | |
| 589 | CR³ | CR⁴ | H | H | 3,3-dimethyl-2- | H | H | H | H | F | H | bond | — | cyclopropyl | 1.92* |
| | | | | | oxobutyl | | | | | | | | | | |
| 590 | CR³ | CR⁴ | H | H | COMe | H | H | H | H | F | H | bond | — | cyclopropyl | 1.53* |
| 591 | CR³ | CR⁴ | H | H | H | COMe | H | H | H | F | H | bond | — | cyclopropyl | 1.45* |
| 592 | CR³ | CR⁴ | H | H | 2-oxo-1,3- | H | H | H | H | F | H | bond | — | cyclopropyl | 1.23* |
| | | | | | oxazolidin-3-yl | | | | | | | | | | |
| 593 | CR³ | CR⁴ | H | H | 2-oxopyrrolidin- | H | H | H | H | F | H | bond | — | cyclopropyl | 1.35* |
| | | | | | 1-yl | | | | | | | | | | |
| 594 | CR³ | CR⁴ | H | H | (methoxycarbonyl)- | H | H | H | H | F | H | bond | — | cyclopropyl | 1.28* |
| | | | | | amino | | | | | | | | | | |
| 595 | CR³ | CR⁴ | H | H | (ethoxycarbonyl)- | H | H | H | H | F | H | bond | — | cyclopropyl | 1.46* |
| | | | | | amino | | | | | | | | | | |
| 596 | CR³ | CR⁴ | H | H | H | (ethoxycarbonyl)- | H | H | H | F | H | bond | — | cyclopropyl | 1.53* |
| | | | | | | amino | | | | | | | | | |
| 597 | CR³ | CR⁴ | H | H | H | (methylcarbamoyl)- | H | H | H | F | H | bond | — | cyclopropyl | 1.28* |
| | | | | | | amino | | | | | | | | | |
| 598 | CR³ | CR⁴ | H | H | H | (methoxycarbonyl)- | H | H | H | F | H | bond | — | cyclopropyl | 1.37* |
| | | | | | | amino | | | | | | | | | |
| 599 | CR³ | CR⁴ | H | H | H | 2-oxopyrrolidin- | H | H | H | F | H | bond | — | cyclopropyl | 1.37* |
| | | | | | | 1-yl | | | | | | | | | |
| 600 | CR³ | CR⁴ | H | H | H | (3-fluoro-2,2- | H | H | H | F | H | bond | — | cyclopropyl | 1.55* |
| | | | | | | dimethylpropanoyl)- | | | | | | | | | |
| | | | | | | amino | | | | | | | | | |
| 601 | CR³ | CR⁴ | H | H | Me | NHCOMe | H | H | H | F | H | bond | — | cyclopropyl | 1.15* |
| 602 | CR³ | CR⁴ | H | F | H | F | H | H | H | Br | H | bond | — | cyclopropyl | 3.35* |
| 603 | CR³ | CR⁴ | H | F | H | F | H | H | H | Cl | H | methylene | — | cyclopropyl | 3.42* |
| 604 | CR³ | CR⁴ | H | H | H | OMe | H | H | H | CN | H | bond | — | cyclopropyl | 2.48* |
| 605 | CR³ | CR⁴ | H | H | methylsulphonyl | H | H | H | H | CN | H | bond | — | cyclopropyl | 1.92* |
| 606 | CR³ | CR⁴ | H | H | H | 2-methoxy-2- | H | H | H | CN | H | bond | — | cyclopropyl | 2.39* |
| | | | | | | oxoethyl | | | | | | | | | |
| 607 | CR³ | CR⁴ | H | H | H | propionylamino | H | H | H | CN | H | bond | — | cyclopropyl | 1.94* |
| 608 | CR³ | CR⁴ | H | H | H | methylcarbamoyl | H | H | H | CN | H | bond | — | cyclopropyl | 1.64* |
| 609 | CR³ | CR⁴ | H | H | H | SMe | H | H | H | CN | H | bond | — | cyclopropyl | 2.92* |
| 610 | CR³ | CR⁴ | H | H | H | COMe | H | H | H | CN | H | bond | — | cyclopropyl | 2.23* |
| 611 | CR³ | CR⁴ | H | H | H | (methylcarbamoyl)- | H | H | H | CN | H | bond | — | cyclopropyl | 1.92* |
| | | | | | | oxy | | | | | | | | | |
| 612 | CR³ | CR⁴ | H | H | H | (methoxycarbonyl)- | H | H | H | CN | H | bond | — | cyclopropyl | 2.06* |
| | | | | | | amino | | | | | | | | | |
| 613 | CR³ | CR⁴ | H | H | —NHCOCH₂— | | H | H | H | CN | H | bond | | cyclopropyl | 1.5* |
| 614 | CR³ | CR⁴ | H | H | H | dimethylcarbamoyl | H | H | H | CN | H | bond | — | cyclopropyl | 1.83* |
| 615 | CR³ | CR⁴ | H | H | CH₂NHCOOtBu | H | H | H | H | CN | H | bond | — | cyclopropyl | 2.77* |
| 616 | CR³ | CR⁴ | H | H | F | H | H | H | H | CN | H | bond | — | cyclopropyl | 2.58* |
| 617 | CR³ | CR⁴ | H | H | H | SO₂NMe₂ | H | H | H | CN | H | bond | — | cyclopropyl | 2.36* |
| 618 | CR³ | CR⁴ | H | H | H | 3-methyl-2- | H | H | H | Cl | H | methylene | — | cyclopropyl | 1.58* |
| | | | | | | oxoimidazolidin- | | | | | | | | | |
| | | | | | | 1-yl | | | | | | | | | |
| 619 | CR³ | CR⁴ | H | H | H | pyrrolidin-1-yl | H | H | H | Cl | H | bond | — | cyclopropyl | 2.23** |
| 620 | CR³ | CR⁴ | H | H | H | F | H | H | H | Cl | H | bond | — | cyclopropyl | 3.15* |
| 621 | CR³ | CR⁴ | H | H | H | pyrrolidin-1-yl | H | H | H | Br | H | bond | — | cyclopropyl | 2.38** |
| 622 | CR³ | CR⁴ | H | H | H | (2,2,2-tri- | H | H | H | Br | H | bond | — | cyclopropyl | 1.91* |
| | | | | | | fluoroethyl)- | | | | | | | | | |
| | | | | | | carbamoyl | | | | | | | | | |
| 623 | CR³ | CR⁴ | H | H | (methoxycarbonyl)- | H | F | H | H | Br | H | bond | — | cyclopropyl | 1.69* |
| | | | | | amino | | | | | | | | | | |
| 624 | CR³ | CR⁴ | H | H | H | 2,2,2-trifluoro-1- | H | H | H | CF₃ | H | bond | — | cyclopropyl | 3.74* |
| | | | | | | methoxyethyl | | | | | | | | | |
| 625 | CR³ | CR⁴ | H | F | H | 2-oxopyrrolidin- | H | H | H | Cl | H | bond | — | cyclopropyl | 1.83** |
| | | | | | | 1-yl | | | | | | | | | |
| 626 | CR³ | CR⁴ | H | H | SMe | Cl | H | H | H | Cl | H | methylene | — | cyclopropyl | 3.15* |
| 627 | CR³ | CR⁴ | H | H | H | ethynyl | H | H | H | CF₃ | H | bond | — | cyclopropyl | 3.36* |
| 628 | CR³ | CR⁴ | H | H | H | cyclopropyl- | H | H | H | Br | H | bond | — | cyclopropyl | 1.49* |
| | | | | | | carbamoyl | | | | | | | | | |
| 629 | CR³ | CR⁴ | H | H | H | cyclopropyl- | H | H | H | Cl | H | bond | — | cyclopropyl | 1.39* |
| | | | | | | carbamoyl | | | | | | | | | |
| 630 | CR³ | CR⁴ | H | H | H | (2,2,2-trifluoro- | H | H | H | Cl | H | bond | — | cyclopropyl | 1.8* |
| | | | | | | ethyl)carbamoyl | | | | | | | | | |
| 631 | CR³ | CR⁴ | H | H | H | ethylcarbamoyl | H | H | H | Cl | H | bond | — | cyclopropyl | 1.35* |
| 632 | CR³ | CR⁴ | H | H | (isopropylcarbamoyl)- | H | H | H | H | Cl | H | bond | — | cyclopropyl | 1.32** |
| | | | | | amino | | | | | | | | | | |
| 633 | CR³ | CR⁴ | H | H | H | isopropyl | H | H | H | Cl | H | bond | — | cyclopropyl | 2.63* |
| 634 | CR³ | CR⁴ | H | H | H | Et | H | H | H | Cl | H | bond | — | cyclopropyl | 2.35* |
| 635 | CR³ | CR⁴ | H | H | F | OMe | H | H | H | CF₃ | H | bond | — | cyclopropyl | 2.99* |
| 636 | CR³ | CR⁴ | H | H | Cl | 2,5-dioxopyrrolidin- | H | H | H | Cl | H | bond | — | cyclopropyl | 1.74* |
| | | | | | | 1-yl | | | | | | | | | |
| 637 | CR³ | CR⁴ | H | H | H | 2-oxopyrrolidin- | H | H | H | I | H | bond | — | cyclopropyl | 1.6* |
| | | | | | | 1-yl | | | | | | | | | |
| 638 | CR³ | CR⁴ | H | H | H | CN | H | H | H | CF₃ | H | bond | — | cyclopropyl | 2.95* |
| 639 | CR³ | CR⁴ | H | H | NHCOMe | OMe | H | H | H | CF₃ | H | bond | — | cyclopropyl | 1.85* |
| 640 | CR³ | CR⁴ | H | H | H | OMe | H | H | H | CF₃ | H | bond | — | cyclopropyl | 2.73* |
| 641 | CR³ | CR⁴ | H | H | OMe | H | H | H | H | CF₃ | H | bond | — | cyclopropyl | 2.21*; |
| | | | | | | | | | | | | | | | 2.41** |
| 642 | CR³ | CR⁴ | H | H | CN | H | H | H | H | CF₃ | H | bond | — | cyclopropyl | 2.99* |
| 643 | CR³ | CR⁴ | H | H | H | trifluoromethoxy | H | H | H | CF₃ | H | bond | — | cyclopropyl | 4.08* |
| 644 | CR³ | CR⁴ | H | H | methylsulphonyl | H | H | H | H | CF₃ | H | bond | — | cyclopropyl | 2.36* |
| 645 | CR³ | CR⁴ | H | H | SO₂NMe₂ | H | H | H | H | CF₃ | H | bond | — | cyclopropyl | 2.84* |
| 646 | CR³ | CR⁴ | H | H | SMe | Cl | H | H | H | CF₃ | H | bond | — | cyclopropyl | 4.08* |
| 647 | CR³ | CR⁴ | H | H | NHCOtBu | H | H | H | H | CF₃ | H | bond | — | cyclopropyl | 2.46* |
| 648 | CR³ | CR⁴ | H | H | NHCOMe | Cl | H | H | H | CF₃ | H | bond | — | cyclopropyl | 2.18*; |
| | | | | | | | | | | | | | | | 2.27** |
| 649 | CR³ | CR⁴ | H | H | H | NHCOMe | H | H | H | CF₃ | H | bond | — | cyclopropyl | 1.78*; |
| | | | | | | | | | | | | | | | 1.77** |
| 650 | CR³ | CR⁴ | H | H | H | NHCOtBu | H | H | H | CF₃ | H | bond | — | cyclopropyl | 2.6* |
| 651 | CR³ | CR⁴ | H | H | methylcarbamoyl | H | H | H | H | CF₃ | H | bond | — | cyclopropyl | 1.78*; |
| | | | | | | | | | | | | | | | 1.81** |
| 652 | CR³ | CR⁴ | H | H | H | methylcarbamoyl | H | H | H | CF₃ | H | bond | — | cyclopropyl | 1.78*; |
| | | | | | | | | | | | | | | | 1.78** |
| 653 | CR³ | CR⁴ | H | H | SMe | H | H | H | H | CF₃ | H | bond | — | cyclopropyl | 3.1*; |
| | | | | | | | | | | | | | | | 3.35** |
| 654 | CR³ | CR⁴ | H | H | H | piperidin-1- | H | H | H | CF₃ | H | bond | — | cyclopropyl | 2.77* |
| | | | | | | ylsulphonyl | | | | | | | | | |
| 655 | CR³ | CR⁴ | H | H | 3,3-dimethyl-2- | H | H | H | H | CF₃ | H | bond | — | cyclopropyl | 3.33* |
| | | | | | oxobutyl | | | | | | | | | | |
| 656 | CR³ | CR⁴ | H | H | COMe | H | H | H | H | CF₃ | H | bond | — | cyclopropyl | 2.7*; |
| | | | | | | | | | | | | | | | 2.8** |
| 657 | CR³ | CR⁴ | H | H | H | COMe | H | H | H | CF₃ | H | bond | — | cyclopropyl | 2.57*; |
| | | | | | | | | | | | | | | | 2.72** |
| 658 | CR³ | CR⁴ | H | H | 2,5-dioxoimidazolidin- | H | H | H | H | CF₃ | H | bond | — | cyclopropyl | 1.58* |
| | | | | | 4-yl | | | | | | | | | | |
| 659 | CR³ | CR⁴ | H | H | H | (methylcarbamoyl)- | H | H | H | CF₃ | H | bond | — | cyclopropyl | 2.06*; |
| | | | | | | oxy | | | | | | | | | 2.13** |
| 660 | CR³ | CR⁴ | H | H | H | (methoxycarbonyl)- | H | H | H | CF₃ | H | bond | — | cyclopropyl | 2.16*; |
| | | | | | | amino | | | | | | | | | 2.06** |
| 661 | CR³ | CR⁴ | H | H | —CH₂CH₂N(COCH₃)— | | H | H | H | CF₃ | H | bond | | cyclopropyl | 2.04* |
| 662 | CR³ | CR⁴ | H | H | Me | NHCOMe | H | H | H | CF₃ | H | bond | — | cyclopropyl | 1.71*; |
| | | | | | | | | | | | | | | | 1.82** |
| 663 | CR³ | CR⁴ | H | H | NHCOisoPr | H | H | H | H | CF₃ | H | bond | — | cyclopropyl | 2.11* |
| 664 | CR³ | CR⁴ | H | H | SMe | amino | H | H | H | Cl | H | bond | — | cyclopropyl | 1.69* |
| 665 | CR³ | CR⁴ | H | H | H | (1-methylpropyl)- | H | H | H | Cl | H | bond | — | cyclopropyl | 3.69* |
| | | | | | | sulphanyl | | | | | | | | | |
| 666 | CR³ | CR⁴ | H | H | H | 2,2,2-trifluoro-1- | H | H | H | CF₃ | H | bond | — | cyclopropyl | 2.69* |
| | | | | | | hydroxyethyl | | | | | | | | | |
| 667 | CR³ | CR⁴ | H | H | H | 2,2,2-trifluoro-1- | H | H | H | CF₃ | H | methylene | — | cyclopropyl | 2.82* |
| | | | | | | hydroxyethyl | | | | | | | | | |
| 668 | CR³ | CR⁴ | H | H | H | OMe | H | H | H | Cl | H | methylene | — | cyclopropyl | 1.97* |
| 669 | CR³ | CR⁴ | H | H | OMe | H | H | H | H | Cl | H | methylene | — | cyclopropyl | 1.73* |
| 670 | CR³ | CR⁴ | H | H | methylsulphonyl | H | H | H | H | Cl | H | methylene | — | cyclopropyl | 1.97* |
| 671 | CR³ | CR⁴ | H | H | acetyl(methyl)amino | H | H | H | H | Cl | H | methylene | — | cyclopropyl | 1.58* |
| 672 | CR³ | CR⁴ | H | H | (trifluoroacetyl)- | H | H | H | H | Cl | H | methylene | — | cyclopropyl | 2.02* |
| | | | | | amino | | | | | | | | | | |
| 673 | CR³ | CR⁴ | H | H | tert- | H | H | H | H | Cl | H | methylene | — | cyclopropyl | 2.21* |
| | | | | | butylcarbamoyl | | | | | | | | | | |
| 674 | CR³ | CR⁴ | H | H | SMe | H | H | H | H | Cl | H | methylene | — | cyclopropyl | 2.18* |
| 675 | CR³ | CR⁴ | H | H | H | piperidin-1- | H | H | H | Cl | H | methylene | — | cyclopropyl | 2.16* |
| | | | | | | ylsulphonyl | | | | | | | | | |
| 676 | CR³ | CR⁴ | H | H | 3,3-dimethyl-2- | H | H | H | H | Cl | H | methylene | — | cyclopropyl | 2.39* |
| | | | | | oxobutyl | | | | | | | | | | |
| 677 | CR³ | CR⁴ | H | H | propionyl | H | H | H | H | Cl | H | methylene | — | cyclopropyl | 2.55* |
| 678 | CR³ | CR⁴ | H | H | COMe | H | H | H | H | Cl | H | methylene | — | cyclopropyl | 2.13* |
| 679 | CR³ | CR⁴ | H | H | Me | NHCOMe | H | H | H | Cl | H | methylene | — | cyclopropyl | 1.49* |
| 680 | CR³ | CR⁴ | H | H | hydroxyl | H | H | H | H | Cl | H | methylene | — | cyclopropyl | 1.36* |
| 681 | CR³ | CR⁴ | H | H | H | hydroxyl | H | H | H | Cl | H | methylene | — | cyclopropyl | 1.49* |
| 682 | CR³ | CR⁴ | Me | H | H | H | Me | H | H | Cl | H | methylene | — | cyclopropyl | 1.87* |
| 683 | CR³ | CR⁴ | H | H | F | H | F | H | H | Cl | H | methylene | — | cyclopropyl | 2.21* |
| 684 | CR³ | CR⁴ | H | H | CF₃ | H | H | H | H | Cl | H | methylene | — | cyclopropyl | 3.33* |
| 685 | CR³ | CR⁴ | H | H | H | CN | H | H | H | Cl | H | methylene | — | cyclopropyl | 2.26* |
| 686 | CR³ | CR⁴ | H | H | OMe | OMe | H | H | H | Cl | H | methylene | — | cyclopropyl | 1.63* |
| 687 | CR³ | CR⁴ | H | H | H | H | OMe | H | H | Cl | H | methylene | — | cyclopropyl | 1.99* |
| 688 | CR³ | CR⁴ | H | H | trifluoromethoxy | H | H | H | H | Cl | H | methylene | — | cyclopropyl | 2.95* |
| 689 | CR³ | CR⁴ | H | H | 1,3-thiazol-4-yl | H | H | H | H | Cl | H | methylene | — | cyclopropyl | 2.06* |
| 690 | CR³ | CR⁴ | H | Cl | H | Cl | H | H | H | Cl | H | methylene | — | cyclopropyl | 4.3* |
| 691 | CR³ | CR⁴ | H | H | methoxycarbonyl | Cl | H | H | H | Cl | H | methylene | — | cyclopropyl | 3.1* |
| 692 | CR³ | CR⁴ | H | H | H | ethoxy | H | H | H | Cl | H | methylene | — | cyclopropyl | 2.21* |
| 693 | CR³ | CR⁴ | H | H | Cl | SO₂NH₂ | H | H | H | Cl | H | methylene | — | cyclopropyl | 1.83* |
| 694 | CR³ | CR⁴ | H | H | hydroxyl | H | F | H | H | Cl | H | methylene | — | cyclopropyl | 1.46* |
| 695 | CR³ | CR⁴ | H | H | methylenebis(oxy) | | H | H | H | Cl | H | methylene | — | cyclopropyl | 1.75* |
| 696 | CR³ | CR⁴ | H | H | morpholin-4- | H | H | H | H | Cl | H | methylene | — | cyclopropyl | 1.59* |
| | | | | | ylcarbonyl | | | | | | | | | | |
| 697 | CR³ | CR⁴ | H | H | (methylsulphonyl)- | H | H | H | H | Cl | H | methylene | — | cyclopropyl | 1.51* |
| | | | | | amino | | | | | | | | | | |
| 698 | CR³ | CR⁴ | H | H | H | 2-(tert-butylamino)- | H | H | H | Br | H | bond | — | cyclopropyl | 1.94* |
| | | | | | | 2-oxoethyl | | | | | | | | | |
| 699 | CR³ | CR⁴ | H | H | H | propoxy | H | H | H | CF₃ | H | bond | — | cyclopropyl | 3.92* |
| 700 | CR³ | CR⁴ | H | H | Cl | OMe | H | H | H | CF₃ | H | bond | — | cyclopropyl | 3.59* |
| 701 | CR³ | CR⁴ | H | H | H | diethylcarbamoyl | H | H | H | Cl | H | bond | — | cyclopropyl | 1.86* |
| 702 | CR³ | CR⁴ | H | H | H | ethylcarbamoyl | H | H | H | Br | H | bond | — | cyclopropyl | 1.44* |
| 703 | CR³ | CR⁴ | H | H | H | 1-chloroethyl | H | H | H | CF₃ | H | methylene | — | cyclopropyl | 3.89* |
| 704 | CR³ | CR⁴ | H | H | piperidin-1- | H | H | H | H | CF₃ | H | methylene | — | cyclopropyl | 3.04* |
| | | | | | ylcarbonyl | | | | | | | | | | |
| 705 | CR³ | CR⁴ | H | H | H | 1-hydroxyethyl | H | H | H | CF₃ | H | methylene | — | cyclopropyl | 2.16* |
| 706 | CR³ | CR⁴ | H | H | H | propylthio | H | H | H | Br | H | bond | — | cyclopropyl | 3.49* |
| 707 | CR³ | CR⁴ | H | H | H | isopropylthio | H | H | H | Br | H | bond | — | cyclopropyl | 3.38* |
| 708 | CR³ | CR⁴ | H | H | H | ethylthio | H | H | H | Br | H | bond | — | cyclopropyl | 2.92* |
| 709 | CR³ | CR⁴ | H | H | H | methoxymethyl | H | H | H | Cl | H | methylene | — | cyclopropyl | 1.82* |
| 710 | CR³ | CR⁴ | H | H | propionylamino | H | H | H | H | CF₃ | H | bond | — | cyclopropyl | 1.93* |
| 711 | CR³ | CR⁴ | H | H | propionylamino | H | H | H | H | Cl | H | bond | — | cyclopropyl | 1.24* |
| 712 | CR³ | CR⁴ | H | H | H | cyanomethyl | H | H | H | Cl | H | bond | — | cyclopropyl | 1.66* |
| 713 | CR³ | CR⁴ | H | H | H | 2-oxopiperidin-1-yl | H | H | H | Cl | H | bond | — | cyclopropyl | 1.47* |
| 714 | CR³ | CR⁴ | H | H | H | 2-furyl | H | H | H | Cl | H | bond | — | cyclopropyl | 2.54* |
| 715 | CR³ | CR⁴ | H | H | H | allyloxy | H | H | H | Cl | H | bond | — | cyclopropyl | 2.37* |
| 716 | CR³ | CR⁴ | H | H | H | formamido | H | H | H | Br | H | bond | — | cyclopropyl | 1.23* |
| 717 | CR³ | CR⁴ | H | H | H | (phenylcarbomoyl)- | H | H | H | Cl | H | bond | — | cyclopropyl | 1.93* |
| | | | | | | amino | | | | | | | | | |
| 718 | CR³ | CR⁴ | H | H | H | (dimethylamino)- | H | H | H | Cl | H | bond | — | cyclopropyl | 0.79* |
| | | | | | | methyl | | | | | | | | | |
| 719 | CR³ | CR⁴ | H | OMe | OMe | OMe | H | H | H | CF₃ | H | bond | — | cyclopropyl | 2.56** |
| 720 | CR³ | CR⁴ | H | H | H | 1H-imidazol-1-yl | H | H | H | Cl | H | bond | — | cyclopropyl | 0.91* |
| 721 | CR³ | CR⁴ | H | H | H | methoxymethyl | H | H | H | CF₃ | H | bond | — | cyclopropyl | 2.78* |
| 722 | CR³ | CR⁴ | H | H | H | H | H | H | H | CHF₂ | H | bond | — | cyclopropyl | 1.56* |
| 723 | CR³ | CR⁴ | H | H | piperidin-1- | H | H | H | H | Cl | H | bond | — | cyclopropyl | 1.97* |
| | | | | | ylcarbonyl | | | | | | | | | | |
| 724 | CR³ | CR⁴ | H | H | H | methoxymethyl | H | H | H | CHF₂ | H | bond | — | cyclopropyl | 1.58* |
| 725 | CR³ | CR⁴ | H | H | piperidin-1- | H | H | H | H | CF₃ | H | bond | — | cyclopropyl | 2.86* |
| | | | | | ylcarbonyl | | | | | | | | | | |
| 726 | CR³ | CR⁴ | H | H | H | 2-oxopyrridin- | H | H | H | Cl | H | bond | — | cyclopropyl | 1.39* |
| | | | | | | 1 (2H)-yl | | | | | | | | | |
| 727 | CR³ | CR⁴ | H | H | H | piperidin-4-yl | H | H | H | Cl | H | bond | — | cyclopropyl | 0.88* |
| 728 | CR³ | CR⁴ | H | H | (ethoxycarbonyl)- | hydroxy | H | H | H | Br | H | bond | — | cyclopropyl | 1.53* |
| | | | | | amino | | | | | | | | | | |
| 729 | CR³ | CR⁴ | H | H | isopropyl | H | H | H | H | Cl | H | bond | — | cyclopropyl | 2.54* |
| 730 | CR³ | CR⁴ | H | H | H | Me | H | H | H | Br | H | bond | — | cyclopropyl | 2.07* |
| 731 | CR³ | CR⁴ | H | H | H | Me | H | H | H | Cl | H | bond | — | cyclopropyl | 1.93* |
| 732 | CR³ | CR⁴ | H | H | H | (1E)-N-methoxy- | H | H | H | Cl | H | bond | — | cyclopropyl | 2.37* |
| | | | | | | ethaneimidoyl | | | | | | | | | |
| 733 | CR³ | CR⁴ | H | H | H | 2-oxopiperidin-1-yl | H | H | H | Br | H | bond | — | cyclopropyl | 1.55* |
| 734 | CR³ | CR⁴ | H | H | H | H | H | H | H | Cl | Me | bond | — | cyclopropyl | 2.7** |
| 735 | CR³ | CR⁴ | H | H | H | morpholin-4- | H | H | H | Cl | H | bond | — | cyclopropyl | 1.33* |
| | | | | | | ylcarbonyl | | | | | | | | | |
| 736 | CR³ | CR⁴ | H | H | H | (1Z)-N-hydroxy- | H | H | H | Cl | H | bond | — | cyclopropyl | 1.52* |
| | | | | | | ethaneimido | | | | | | | | | |
| 737 | CR³ | CR⁴ | H | H | H | SMe | H | H | H | H | Me | bond | — | cyclopropyl | 1.49* |
| 738 | CR³ | CR⁴ | H | H | H | SMe | H | H | H | Cl | Me | bond | — | cyclopropyl | 3.59** |
| 739 | CR³ | CR⁴ | H | H | (phenylcarbamoyl)- | H | H | H | H | Cl | H | bond | — | cyclopropyl | 1.75* |
| | | | | | amino | | | | | | | | | | |
| 740 | CR³ | CR⁴ | H | H | CF₃ | H | H | H | H | Br | H | bond | — | cyclopropyl | 3.33* |
| 741 | CR³ | CR⁴ | H | H | H | CN | H | H | H | Br | H | bond | — | cyclopropyl | 2.26* |
| 742 | CR³ | CR⁴ | H | H | OMe | OMe | H | H | H | Br | H | bond | — | cyclopropyl | 1.53* |
| 743 | CR³ | CR⁴ | H | H | Br | H | H | H | H | Br | H | bond | — | cyclopropyl | 2.7* |
| 744 | CR³ | CR⁴ | H | H | CN | H | H | H | H | Br | H | bond | — | cyclopropyl | 2.43* |
| 745 | CR³ | CR⁴ | H | H | (trifluoromethyl)- | H | H | H | H | Br | H | bond | — | cyclopropyl | 3.9* |
| | | | | | thio | | | | | | | | | | |
| 746 | CR³ | CR⁴ | H | H | SMe | Cl | H | H | H | Br | H | bond | — | cyclopropyl | 3.03* |
| 747 | CR³ | CR⁴ | H | H | 1,3-thiazol-4-yl | H | H | H | H | Br | H | bond | — | cyclopropyl | 1.99* |
| 748 | CR³ | CR⁴ | H | H | difluoromethoxy | H | H | H | H | Br | H | bond | — | cyclopropyl | 2.21* |
| 749 | CR³ | CR⁴ | H | Cl | H | Cl | H | H | H | Br | H | bond | — | cyclopropyl | 4.43* |
| 750 | CR³ | CR⁴ | H | H | H | ethoxycarbonyl | H | H | H | Br | H | bond | — | cyclopropyl | 2.4* |
| 751 | CR³ | CR⁴ | H | H | H | ethoxy | H | H | H | Br | H | bond | — | cyclopropyl | 2.18* |
| 752 | CR³ | CR⁴ | H | H | methylenebis(oxy) | | H | H | H | Br | H | bond | — | cyclopropyl | 1.66* |
| 753 | CR³ | CR⁴ | H | H | trifluoromethoxy | Cl | H | H | H | Br | H | bond | — | cyclopropyl | 4.13* |
| 754 | CR³ | CR⁴ | H | H | H | acryloylamino | H | H | H | Br | H | bond | — | cyclopropyl | 1.54* |
| 755 | CR³ | CR⁴ | H | H | allylcarbamoyl | H | H | H | H | Br | H | bond | — | cyclopropyl | 1.73* |
| 756 | CR³ | CR⁴ | H | H | (methylsulphonyl)- | H | H | H | H | Br | H | bond | — | cyclopropyl | 1.44* |
| | | | | | amino | | | | | | | | | | |
| 757 | CR³ | CR⁴ | H | H | [(methylamino)- | H | H | H | H | Br | H | bond | — | cyclopropyl | 1.51* |
| | | | | | sulphonyl]methyl | | | | | | | | | | |
| 758 | CR³ | CR⁴ | H | H | 2-ethoxy-2-oxoethyl | H | H | H | H | Br | H | bond | — | cyclopropyl | 2.06* |
| 759 | CR³ | CR⁴ | H | H | cyanoacetyl | H | H | H | H | Br | H | bond | — | cyclopropyl | 2.16* |
| 760 | CR³ | CR⁴ | H | H | hydroxyl | propionyl | H | H | H | Br | H | bond | — | cyclopropyl | 2.06* |
| 761 | CR³ | CR⁴ | H | OMe | OMe | OMe | H | H | H | Br | H | bond | — | cyclopropyl | 1.71* |
| 762 | CR³ | CR⁴ | H | H | methoxycarbonyl | H | H | H | H | Br | H | bond | — | cyclopropyl | 2.4* |
| 763 | CR³ | CR⁴ | H | H | hydroxyl | COMe | H | H | H | Br | H | bond | — | cyclopropyl | 1.75* |
| 764 | CR³ | CR⁴ | H | H | hydroxyl | F | H | H | H | Br | H | bond | — | cyclopropyl | 1.41* |
| 765 | CR³ | CR⁴ | H | H | H | hydroxyl | H | H | H | Br | H | bond | — | cyclopropyl | 1.39* |
| 766 | CR³ | CR⁴ | H | H | H | hydroxymethyl | H | H | H | Br | H | bond | — | cyclopropyl | 1.37* |
| 767 | CR³ | CR⁴ | H | H | hydroxyl | propionylamino | H | H | H | Br | H | bond | — | cyclopropyl | 1.85* |
| 768 | CR³ | CR⁴ | H | H | H | carboxymethyl | H | H | H | Cl | H | bond | — | cyclopropyl | 1.23* |
| 769 | CR³ | CR⁴ | H | H | H | 2-oxopyrrolidin- | H | H | H | Cl | Me | bond | — | cyclopropyl | 2.22** |
| | | | | | | 1-yl | | | | | | | | | |
| 770 | CR³ | CR⁴ | H | H | H | piperidin-1- | H | H | H | Cl | H | bond | — | cyclopropyl | 1.94* |
| | | | | | | ylcarbonyl | | | | | | | | | |
| 771 | CR³ | CR⁴ | H | H | F | 2-oxopyrrolidin- | H | H | H | Br | H | bond | — | cyclopropyl | 1.61** |
| | | | | | | 1-yl | | | | | | | | | |
| 772 | CR³ | CR⁴ | H | H | H | methoxymethyl | H | H | H | CHF₂ | H | methylene | — | cyclopropyl | 1.73* |
| 773 | CR³ | CR⁴ | H | H | H | H | H | H | H | CHF₂ | H | methylene | — | cyclopropyl | 1.68* |
| 774 | CR³ | CR⁴ | H | H | H | 2-oxopyrrolidin- | H | H | H | CHF₂ | H | methylene | — | cyclopropyl | 1.48* |
| | | | | | | 1-yl | | | | | | | | | |
| 775 | CR³ | CR⁴ | H | H | H | Et | H | H | H | Br | H | bond | — | cyclopropyl | 2.44* |
| 776 | CR³ | CR⁴ | H | H | H | Et | H | H | H | Cl | H | methylene | — | cyclopropyl | 2.37* |
| 777 | CR³ | CR⁴ | H | H | H | isopropyl | H | H | H | Cl | H | methylene | — | cyclopropyl | 2.71* |
| 778 | CR³ | CR⁴ | H | H | H | tert-butyl | H | H | H | Cl | H | bond | — | cyclopropyl | 2.9* |
| 779 | CR³ | CR⁴ | H | H | H | tert-butyl | H | H | H | Br | H | bond | — | cyclopropyl | 3.14* |
| 780 | CR³ | CR⁴ | H | H | H | 2-oxopropyl | H | H | H | Cl | H | bond | — | cyclopropyl | 1.51* |
| 781 | CR³ | CR⁴ | H | H | H | 2-oxopropyl | H | H | H | Cl | H | methylene | — | cyclopropyl | 1.67* |
| 782 | CR³ | CR⁴ | H | H | H | 2-oxopropyl | H | H | H | Br | H | bond | — | cyclopropyl | 1.64* |
| 783 | CR³ | CR⁴ | H | H | H | 2-oxopropyl | H | H | H | CF₃ | H | bond | — | cyclopropyl | 2.42* |
| 784 | CR³ | CR⁴ | H | H | H | 1-hydroxy-1- | H | H | H | Cl | H | bond | — | cyclopropyl | 1.47* |
| | | | | | | methylethyl | | | | | | | | | |
| 785 | CR³ | CR⁴ | H | H | H | COOH | H | H | H | Cl | H | bond | — | cyclopropyl | 1.32* |
| 786 | CR³ | CR⁴ | H | H | H | 3-oxomorpholin- | H | H | H | Cl | H | methylene | — | cyclopropyl | 1.31* |
| | | | | | | 4-yl | | | | | | | | | |
| 787 | CR³ | CR⁴ | H | H | F | 2-oxopyrrolidin- | H | H | H | I | H | bond | — | cyclopropyl | 1.61** |
| | | | | | | 1-yl | | | | | | | | | |
| 788 | CR³ | CR⁴ | H | H | H | nitro | H | H | H | Cl | H | bond | — | cyclopropyl | 2.63* |
| 789 | CR³ | CR⁴ | H | H | H | 3-oxomorpholin- | H | H | H | Br | H | bond | — | cyclopropyl | 1.24* |
| | | | | | | 4-yl | | | | | | | | | |
| 790 | CR³ | CR⁴ | H | H | H | 3-oxomorpholin- | H | H | H | CF₃ | H | bond | — | cyclopropyl | 1.88* |
| | | | | | | 4-yl | | | | | | | | | |
| 791 | CR³ | CR⁴ | H | H | H | 3-oxomorpholin- | H | H | H | Cl | H | bond | — | cyclopropyl | 1.2* |
| | | | | | | 4-yl | | | | | | | | | |
| 792 | CR³ | CR⁴ | H | H | H | 2,2,2-trifluoro- | H | H | H | Cl | H | methylene | — | cyclopropyl | 2.74* |
| | | | | | | 1-methoxyethyl | | | | | | | | | |
| 793 | CR³ | CR⁴ | H | H | H | (methoxyacetyl)- | H | H | H | Cl | H | bond | — | cyclopropyl | 1.33* |
| | | | | | | amino | | | | | | | | | |
| 794 | CR³ | CR⁴ | H | H | H | cyanomethyl | H | H | H | Br | H | bond | — | cyclopropyl | 1.75* |
| 795 | CR³ | CR⁴ | H | H | H | 2-carboxyethyl | H | H | H | Cl | H | bond | — | cyclopropyl | 1.09* |
| 796 | CR³ | CR⁴ | H | H | H | (2-methoxy-1- | H | H | H | Cl | H | bond | — | cyclopropyl | 1.47* |
| | | | | | | methylethyl)- | | | | | | | | | |
| | | | | | | carbamoyl | | | | | | | | | |
| 797 | CR³ | CR⁴ | H | H | H | 1-(ethylsulphanyl)- | H | H | H | Br | H | bond | — | cyclopropyl | 3.09* |
| | | | | | | ethyl | | | | | | | | | |
| 798 | CR³ | CR⁴ | H | H | H | 2,2,2-trifluoro-1- | H | H | H | Br | H | bond | — | cyclopropyl | 1.94* |
| | | | | | | hydroxyethyl | | | | | | | | | |
| 799 | CR³ | N | H | —CH═CH—CH═CH— | | — | H | H | H | Cl | H | bond | — | cyclopropyl | 1.84* |
| 800 | CR³ | CR⁴ | H | H | H | H | H | H | H | Cl | H | methylene | — | 2,2- | 2.46* |
| | | | | | | | | | | | | | | dichloro- | |
| | | | | | | | | | | | | | | cyclopropyl | |
| 801 | CR³ | CR⁴ | H | H | H | H | H | H | H | Cl | H | bond | — | 1-(phenoxy- | 2.88* |
| | | | | | | | | | | | | | | methyl- | |
| | | | | | | | | | | | | | | cyclopropyl | |
| 802 | CR³ | CR⁴ | H | H | H | 4-methyl-2-oxo- | H | H | H | F | H | bond | — | cyclopropyl | 1.18* |
| | | | | | | 1,3-oxazolidin-3-yl | | | | | | | | | |
| 803 | CR³ | CR⁴ | H | H | H | 2-oxopiperidin-1-yl | H | H | H | CF₃ | H | bond | — | cyclopropyl | 2.18* |
| 804 | CR³ | CR⁴ | H | H | H | 2-oxopiperidin-1-yl | H | H | H | F | H | bond | — | cyclopropyl | 1.16* |
| 805 | CR³ | CR⁴ | H | H | H | 1-methoxyethyl | H | H | H | Br | H | bond | — | cyclopropyl | 2.08* |
| 806 | CR³ | CR⁴ | H | H | H | 1-methoxyethyl | H | H | H | Cl | H | bond | — | cyclopropyl | 1.96* |
| 807 | CR³ | CR⁴ | H | H | H | 1-(methyl- | H | H | H | Cl | H | bond | — | cyclopropyl | 2.52* |
| | | | | | | sulphanyl)ethyl | | | | | | | | | |
| 808 | CR³ | CR⁴ | H | H | H | H | H | H | H | Br | H | methylene | — | 2,2-dichloro- | 2.6* |
| | | | | | | | | | | | | | | cyclopropyl | |
| 809 | CR³ | CR⁴ | H | H | H | H | H | H | H | Br | H | ethylen | — | 1-cyclopropyl | 2.18* |
| 810 | CR³ | CR⁴ | H | H | H | 1-(ethyl-sulphanyl)- | H | H | H | Cl | H | bond | — | cyclopropyl | 2.82* |
| | | | | | | ethyl | | | | | | | | | |
| 811 | CR³ | CR⁴ | H | H | H | 1-(methyl- | H | H | H | Br | H | bond | — | cyclopropyl | 2.63* |
| | | | | | | sulphanyl)ethyl | | | | | | | | | |
| 812 | CR³ | CR⁴ | H | H | F | cyanomethyl | H | H | H | Br | H | bond | — | cyclopropyl | 1.97* |
| 813 | CR³ | CR⁴ | H | H | F | cyanomethyl | H | H | H | Cl | H | bond | — | cyclopropyl | 1.76* |
| 814 | CR³ | CR⁴ | —N═CH—CH═CH— | | H | H | H | H | H | Cl | H | bond | — | cyclopropyl | 2.97* |
| 815 | CR³ | CR⁴ | H | —CH═CH—CH═N— | | H | H | H | H | Cl | H | bond | — | cyclopropyl | 1.2* |
| 816 | CR³ | CR⁴ | H | H | F | cyanomethyl | H | H | H | Cl | H | methylene | — | cyclopropyl | 1.87* |
| 817 | CR³ | CR⁴ | H | H | cyanomethyl | H | OMe | H | H | Cl | H | bond | — | cyclopropyl | 1.82* |
| 818 | CR³ | CR⁴ | H | H | cyanomethyl | H | OMe | H | H | Br | H | bond | — | cyclopropyl | 1.99* |
| 819 | CR³ | CR⁴ | H | H | Me | cyanomethyl | H | H | H | Cl | H | bond | — | cyclopropyl | 1.79* |
| 820 | CR³ | CR⁴ | H | H | Me | cyanomethyl | H | H | H | Br | H | bond | — | cyclopropyl | 1.85* |
| 821 | CR³ | CR⁴ | H | H | H | cyanomethyl | H | H | H | Cl | H | methylene | — | cyclopropyl | 1.81* |
| 822 | CR³ | CR⁴ | H | H | NHCOMe | hydroxyl | H | H | H | Cl | H | bond | — | cyclopropyl | 1* |
| 823 | CR³ | CR⁴ | H | H | [(2,2-dimethylprop- | H | H | H | H | Cl | H | bond | — | cyclopropyl | 3.78* |
| | | | | | yl)sulphanyl]methyl | | | | | | | | | | |
| 824 | CR³ | CR⁴ | H | H | dimethylcarbamoyl | H | H | H | H | CF₃ | H | bond | — | cyclopropyl | 2.08* |
| 825 | CR³ | CR⁴ | H | H | H | 1-(methyl- | H | H | H | CF₃ | H | bond | — | cyclopropyl | 3.81* |
| | | | | | | sulphanyl)ethyl | | | | | | | | | |
| 826 | CR³ | CR⁴ | H | H | H | 1-methoxyethyl | H | H | H | CF₃ | H | bond | — | cyclopropyl | 3.16* |
| 827 | CR³ | CR⁴ | H | H | H | 1-(methyl- | H | H | H | CF₃ | H | methylene | — | cyclopropyl | 3.95* |
| | | | | | | sulphanyl)ethyl | | | | | | | | | |
| 828 | CR³ | CR⁴ | H | H | 2-oxo-1,3- | H | H | H | H | Cl | Me | bond | — | cyclopropyl | 1.95** |
| | | | | | oxozolidin-3-yl | | | | | | | | | | |
| 829 | CR³ | CR⁴ | H | H | tert-buytl | H | H | H | H | Cl | H | bond | — | cyclopropyl | 2.92* |
| 830 | CR³ | CR⁴ | H | H | Me | H | H | H | H | Cl | H | bond | — | cyclopropyl | 1.95* |
| 831 | CR³ | CR⁴ | H | H | H | tert-butyl- | H | H | H | Cl | H | bond | — | cyclopropyl | 2.07* |
| | | | | | | carbamoyl | | | | | | | | | |
| 832 | CR³ | CR⁴ | H | H | cyanomethyl | H | H | H | H | Cl | H | bond | — | cyclopropyl | 1.59* |
| 833 | CR³ | CR⁴ | H | H | H | 2-oxopyrridin- | H | H | H | Br | H | bond | — | cyclopropyl | 1.46*; |
| | | | | | | 1(2H)-yl | | | | | | | | | 1.46** |
| 834 | CR³ | CR⁴ | H | H | H | 2-oxo-1,3- | H | H | H | CF₃ | H | bond | — | cyclopropyl | 2.18*; |
| | | | | | | oxazolidin-3-yl | | | | | | | | | 2.18** |
| 835 | CR³ | CR⁴ | H | H | H | H | H | H | Me | Cl | H | bond | — | cyclopropyl | 1.65** |
| 836 | CR³ | CR⁴ | H | H | H | H | H | H | H | Br | H | bond | — | cyclopropyl | 2.56* |
| 837 | CR³ | CR⁴ | H | H | H | SMe | H | H | Me | Cl | H | bond | — | cyclopropyl | 2.23** |
| 838 | CR³ | CR⁴ | H | H | H | 2-oxopyrrolidin-1-yl | H | H | Me | Cl | H | bond | — | cyclopropyl | 1.47** |
| 839 | CR³ | CR⁴ | H | H | H | methoxymethyl | H | H | Me | Cl | H | bond | — | cyclopropyl | 1.67** |
| 840 | CR³ | CR⁴ | H | H | 2-oxo-1,3- | H | H | H | Me | Cl | H | bond | — | cyclopropyl | 1.31** |
| | | | | | oxazolidin-3-yl | | | | | | | | | | |
| 841 | CR³ | CR⁴ | H | H | H | 3-oxobutyl | H | H | H | Cl | H | bond | — | cyclopropyl | 1.72* |
| 842 | CR³ | CR⁴ | H | H | H | 2-oxo-1,3- | H | H | H | Br | H | bond | — | cyclopropyl | 1.51* |
| | | | | | | oxazolidin-3-yl | | | | | | | | | |
| 843 | CR³ | CR⁴ | H | H | H | 2-oxo-1,3- | H | H | H | Cl | H | bond | — | cyclopropyl | 1.4* |
| | | | | | | oxazolidin-3-yl | | | | | | | | | |
| 844 | CR³ | CR⁴ | H | H | H | (tert-butylamino)- | H | H | H | Cl | H | bond | — | cyclopropyl | 2.37* |
| | | | | | | sulphonyl | | | | | | | | | |
| 845 | CR³ | CR⁴ | H | Cl | hydroxyl | Cl | H | H | H | Cl | H | bond | — | cyclopropyl | 1.99* |
| 846 | CR³ | CR⁴ | H | H | 4-methylpiperazin- | CF₃ | H | H | H | Cl | H | bond | — | cyclopropyl | 1.38** |
| | | | | | 1-yl | | | | | | | | | | |
| 847 | CR³ | CR⁴ | H | H | H | methoxymethyl | H | H | H | I | H | bond | — | cyclopropyl | 1.86* |
| 848 | CR³ | CR⁴ | H | H | H | pyrrolidin-1- | H | H | H | Cl | H | bond | — | cyclopropyl | 1.57* |
| | | | | | | ylcarbonyl | | | | | | | | | |
| 849 | CR³ | CR⁴ | H | H | H | acetyl(methoxy)- | H | H | H | Br | H | bond | — | cyclopropyl | 1.68** |
| | | | | | | amino | | | | | | | | | |
| 850 | CR³ | CR⁴ | H | H | H | acetyl(methoxy)- | H | H | H | Cl | H | bond | — | cyclopropyl | 1.57** |
| | | | | | | amino | | | | | | | | | |
| 851 | CR³ | CR⁴ | H | H | 2-oxopropyl | H | H | H | H | Cl | H | bond | — | cyclopropyl | 1.48* |
| 852 | CR³ | CR⁴ | H | H | H | propylsulphonyl | H | H | H | Cl | H | bond | — | cyclopropyl | 2.25* |
| 853 | CR³ | CR⁴ | H | H | H | I | H | H | H | Cl | H | bond | — | cyclopropyl | 3.08** |
| 854 | CR³ | CR⁴ | H | H | H | (2E)-2-(methoxy- | H | H | H | Cl | H | bond | — | cyclopropyl | 2.2* |
| | | | | | | imino)propyl | | | | | | | | | |
| 855 | CR³ | CR⁴ | H | H | H | OMe | H | H | H | I | H | bond | — | cyclopropyl | 1.97* |
| 856 | CR³ | CR⁴ | H | H | H | (2-hydroxypropanoyl)- | H | H | H | Cl | H | bond | — | cyclopropyl | 1.49* |
| | | | | | | amino | | | | | | | | | |
| 857 | CR³ | CR⁴ | H | H | H | 2,2,2-trifluoro- | H | H | H | I | H | bond | — | cyclopropyl | 2.82* |
| | | | | | | methoxyethyl | | | | | | | | | |
| 858 | CR³ | CR⁴ | H | H | H | SMe | H | H | H | I | H | bond | — | cyclopropyl | 2.44* |
| 859 | CR³ | CR⁴ | H | I | H | H | H | H | H | Cl | H | bond | TsOH | cyclopropyl | 3.07** |
| 860 | CR³ | CR⁴ | H | H | H | I | H | H | H | Br | H | bond | — | cyclopropyl | 3.34** |
| 861 | CR³ | CR⁴ | H | H | H | 2-oxo-1,3- | H | H | H | Cl | H | methylene | — | cyclopropyl | 1.63* |
| | | | | | | oxazolidin-3-yl | | | | | | | | | |
| 862 | CR³ | CR⁴ | H | H | H | 2-oxo-1,3- | H | H | H | Br | H | methylene | — | cyclopropyl | 1.68* |
| | | | | | | oxazolidin-3-yl | | | | | | | | | |
| 863 | CR³ | CR⁴ | H | H | H | H | OH | H | H | Cl | H | bond | — | cyclopropyl | 1.47* |
| 864 | CR³ | CR⁴ | OH | H | H | 1,1- | H | H | H | Cl | H | bond | — | cyclopropyl | 2.6* |
| | | | | | | dimethylpropyl | | | | | | | | | |
| 865 | CR³ | CR⁴ | OH | H | H | methoxycarbonyl | H | H | H | Cl | H | bond | — | cyclopropyl | 1.66* |
| 866 | CR³ | CR⁴ | OH | H | H | propylsulphonyl | H | H | H | Cl | H | bond | — | cyclopropyl | 1.83* |
| 867 | CR³ | CR⁴ | H | H | 2-oxopyrrolidin- | H | OH | H | H | Cl | H | bond | — | cyclopropyl | 1.44* |
| | | | | | 1-yl | | | | | | | | | | |
| 868 | CR³ | CR⁴ | OH | H | H | phenylsulphonyl | H | H | H | Cl | H | bond | — | cyclopropyl | 2.11* |
| 869 | CR³ | CR⁴ | H | (methyl- | H | H | OH | H | H | Cl | H | bond | — | cyclopropyl | 1.22* |
| | | | | sulphonyl)- | | | | | | | | | | | |
| | | | | methyl | | | | | | | | | | | |
| 870 | CR³ | CR⁴ | H | H | OMe | H | OMe | H | H | Cl | H | bond | — | cyclopropyl | 1.64* |
| 871 | CR³ | CR⁴ | H | H | H | H | Br | H | H | Cl | H | bond | — | cyclopropyl | 2.7* |
| 872 | CR³ | CR⁴ | H | H | H | H | isopro- | H | H | Cl | H | bond | — | cyclopropyl | 1.96* |
| | | | | | | | pyl | | | | | | | | |
| 873 | CR³ | CR⁴ | H | Cl | H | H | phen- | H | H | Cl | H | bond | — | cyclopropyl | 4.87* |
| | | | | | | | oxy | | | | | | | | |
| 874 | CR³ | CR⁴ | H | H | (hydroxyacetyl)- | H | H | H | H | Cl | H | bond | — | cyclopropyl | 0.66* |
| | | | | | amino | | | | | | | | | | |
| 875 | CR³ | CR⁴ | Me | H | benzoylamino | Cl | H | H | H | Cl | H | bond | — | cyclopropyl | 2.43* |
| 876 | CR³ | CR⁴ | H | H | H | H | ethoxy | H | H | Cl | H | bond | — | cyclopropyl | 2.13* |
| 877 | CR³ | CR⁴ | H | H | butyl | H | H | H | H | Cl | H | bond | — | cyclopropyl | 2.88* |
| 878 | CR³ | CR⁴ | H | H | Me | H | Br | H | H | Cl | H | bond | — | cyclopropyl | 2.88* |
| 879 | CR³ | CR⁴ | Me | H | Cl | Me | H | H | H | Cl | H | bond | — | cyclopropyl | 2.43* |
| 880 | CR³ | CR⁴ | H | H | H | H | SMe | H | H | Cl | H | bond | — | cyclopropyl | 2.18* |
| 881 | CR³ | CR⁴ | H | H | H | H | CN | H | H | Cl | H | bond | — | cyclopropyl | 0.85* |
| 882 | CR³ | CR⁴ | H | H | H | Br | H | H | H | Cl | H | bond | — | cyclopropyl | 2.73* |
| 883 | CR³ | CR⁴ | H | H | H | H | tri- | H | H | Cl | H | bond | — | cyclopropyl | 3.07* |
| | | | | | | | fluoro- | | | | | | | | |
| | | | | | | | meth- | | | | | | | | |
| | | | | | | | oxy | | | | | | | | |
| 884 | CR³ | CR⁴ | H | H | H | H | phenyl- | H | H | Cl | H | bond | — | cyclopropyl | 3.94* |
| | | | | | | | thio | | | | | | | | |
| 885 | CR³ | CR⁴ | H | H | H | H | iso- | H | H | Cl | H | bond | — | cyclopropyl | 3.72* |
| | | | | | | | propyl- | | | | | | | | |
| | | | | | | | thio | | | | | | | | |
| 886 | CR³ | CR⁴ | H | H | Br | H | F | H | H | Cl | H | bond | — | cyclopropyl | 3.26* |
| 887 | CR³ | CR⁴ | H | H | F | H | Cl | H | H | Cl | H | bond | — | cyclopropyl | 2.67* |
| 888 | CR³ | CR⁴ | H | H | H | phenoxy | H | H | H | Cl | H | bond | — | cyclopropyl | 2.99* |
| 889 | CR³ | CR⁴ | H | H | phenoxy | H | H | H | H | Cl | H | bond | — | cyclopropyl | 2.6* |
| 890 | CR³ | CR⁴ | H | H | H | H | benzyl | H | H | Cl | H | bond | — | cyclopropyl | 2.21* |
| 891 | CR³ | CR⁴ | H | H | anilinocarbonyl | H | H | H | H | Cl | H | bond | — | cyclopropyl | 1.83* |
| 892 | CR³ | CR⁴ | H | H | H | benzyloxy | H | H | H | Cl | H | bond | — | cyclopropyl | 2.8* |
| 893 | CR³ | CR⁴ | H | H | Br | H | Et | H | H | Cl | H | bond | — | cyclopropyl | 2.57* |
| 894 | CR³ | CR⁴ | Me | H | H | NHCOMe | H | H | H | Cl | H | bond | — | cyclopropyl | 1.27* |
| 895 | CR³ | CR⁴ | H | H | 4-fluorophenoxy | H | H | H | H | Cl | H | bond | — | cyclopropyl | 2.7* |
| 896 | CR³ | CR⁴ | H | H | H | H | 1,1,2,2- | H | H | Cl | H | bond | — | cyclopropyl | 3.14* |
| | | | | | | | tetra- | | | | | | | | |
| | | | | | | | fluoro- | | | | | | | | |
| | | | | | | | ethoxy | | | | | | | | |
| 897 | CR³ | CR⁴ | OMe | H | H | Cl | H | H | H | Cl | H | bond | — | cyclopropyl | 3.03* |
| 898 | CR³ | CR⁴ | H | H | Me | F | H | H | H | Cl | H | bond | — | cyclopropyl | 2.4* |
| 899 | CR³ | CR⁴ | Me | H | OMe | Me | H | H | H | Cl | H | bond | — | cyclopropyl | 1.68* |
| 900 | CR³ | CR⁴ | H | H | H | H | phenyl | H | H | Cl | H | bond | — | cyclopropyl | 2.46* |
| 901 | CR³ | CR⁴ | H | H | H | H | phen- | H | H | Cl | H | bond | — | cyclopropyl | 3.14* |
| | | | | | | | oxy | | | | | | | | |
| 902 | CR³ | CR⁴ | Me | H | trifluoromethoxy | Cl | H | H | H | Cl | H | bond | — | cyclopropyl | 3.85* |
| 903 | CR³ | CR⁴ | H | CF₃ | H | CF₃ | H | H | H | Cl | H | bond | — | cyclopropyl | 4.56* |
| 904 | CR³ | CR⁴ | H | H | H | Br | Me | H | H | Cl | H | bond | — | cyclopropyl | 2.16* |
| 905 | CR³ | CR⁴ | H | H | Et | H | Et | H | H | Cl | H | bond | — | cyclopropyl | 2.16* |
| 906 | CR³ | CR⁴ | H | H | F | trifluoromethoxy | H | H | H | Cl | H | bond | — | cyclopropyl | 3.94* |
| 907 | CR³ | CR⁴ | H | H | H | H | sec- | H | H | Cl | H | bond | — | cyclopropyl | 2.11* |
| | | | | | | | butyl | | | | | | | | |
| 908 | CR³ | CR⁴ | H | H | F | CF₃ | H | H | H | Cl | H | bond | — | cyclopropyl | 3.14* |
| 909 | CR³ | CR⁴ | H | H | butyl | H | Me | H | H | Cl | H | bond | — | cyclopropyl | 2.63* |
| 910 | CR³ | CR⁴ | H | H | H | CF₃ | F | H | H | Cl | H | bond | — | cyclopropyl | 3.41* |
| 911 | CR³ | CR⁴ | H | H | H | H | butyl | H | H | Cl | H | bond | — | cyclopropyl | 2.3* |
| 912 | CR³ | CR⁴ | H | H | H | H | propyl | H | H | Cl | H | bond | — | cyclopropyl | 2.01* |
| 913 | CR³ | CR⁴ | H | H | trifluoromethoxy | Br | H | H | H | Cl | H | bond | — | cyclopropyl | 3.99* |
| 914 | CR³ | CR⁴ | H | H | H | CF₃ | Me | H | H | Cl | H | bond | — | cyclopropyl | 2.4* |
| 915 | CR³ | CR⁴ | H | H | CN | H | Me | H | H | Cl | H | bond | — | cyclopropyl | 2.26* |
| 916 | CR³ | CR⁴ | H | H | Me | Br | H | H | H | Cl | H | bond | — | cyclopropyl | 2.91* |
| 917 | CR³ | CR⁴ | Me | H | Br | F | H | H | H | Cl | H | bond | — | cyclopropyl | 3.26* |
| 918 | CR³ | CR⁴ | Me | H | H | Br | H | H | H | Cl | H | bond | — | cyclopropyl | 0.6* |
| 919 | CR³ | CR⁴ | H | H | Br | Cl | H | H | H | Cl | H | bond | — | cyclopropyl | 3.63* |
| 920 | CR³ | CR⁴ | H | H | H | tert-butyl | H | H | H | CF₃ | H | bond | — | cyclopropyl | 4.42* |
| 921 | CR³ | CR⁴ | H | H | H | tert-butyl | H | H | H | CF₃ | H | methylene | — | cyclopropyl | 4.59* |
| 922 | CR³ | CR⁴ | H | H | H | H | H | H | H | CF₃ | H | methylene | — | cyclopropyl | 2.99* |
| 923 | CR³ | CR⁴ | H | H | butoxycarbonyl | H | H | H | H | Cl | H | bond | — | cyclopropyl | 3.84** |
| 924 | CR³ | CR⁴ | H | H | 2-oxo-1,3- | H | H | H | CF₃ | Cl | H | bond | — | cyclopropyl | 3.19** |
| | | | | | oxazolidin-3-yl | | | | | | | | | | |
| 925 | CR³ | CR⁴ | H | H | dimethylcarbamoyl | H | H | H | H | I | H | bond | — | cyclopropyl | 1.49*** |
| 926 | CR³ | CR⁴ | H | H | H | SMe | H | H | H | Br | Me | bond | — | cyclopropyl | 1.96** |
| 927 | CR³ | CR⁴ | H | H | (methoxycarbonyl)- | H | H | H | H | CF₃ | H | bond | — | cyclopropyl | 2.06** |
| | | | | | amino | | | | | | | | | | |
| 928 | CR³ | CR⁴ | H | H | H | H | H | H | H | Br | Me | bond | — | cyclopropyl | 1.7** |
| 929 | CR³ | CR⁴ | H | H | H | propylsulphinyl | H | H | H | CF₃ | H | bond | — | cyclopropyl | 2.57* |
| 930 | CR³ | CR⁴ | H | H | H | propylsulphonyl | H | H | H | CF₃ | H | bond | — | cyclopropyl | 3.03* |
| 931 | CR³ | CR⁴ | H | H | H | formyl | H | H | H | Cl | H | bond | — | cyclopropyl | 1.77* |
| 932 | CR³ | CR⁴ | H | H | H | H | H | H | H | Br | H | bond | — | 1-(phenoxy- | 3.03* |
| | | | | | | | | | | | | | | methyl)cyclo- | |
| | | | | | | | | | | | | | | propyl | |
| 933 | CR³ | CR⁴ | Me | H | Cl | Br | H | H | H | Cl | H | bond | — | cyclopropyl | 3.46* |
| 934 | CR³ | CR⁴ | H | H | CF₃ | H | F | H | H | Cl | H | bond | — | cyclopropyl | 3.68* |
| 935 | CR³ | CR⁴ | H | Me | H | H | Me | H | H | Cl | H | bond | — | cyclopropyl | 1.83* |
| 936 | CR³ | CR⁴ | H | CN | Cl | H | F | H | H | Cl | H | bond | — | cyclopropyl | 3.33* |
| 937 | CR³ | CR⁴ | H | H | H | Cl | Cl | H | H | Cl | H | bond | — | cyclopropyl | 3.76* |
| 938 | CR³ | CR⁴ | H | H | H | H | Me | H | H | Cl | H | bond | — | cyclopropyl | 1.61* |
| 939 | CR³ | CR⁴ | H | H | Me | Me | H | H | H | Cl | H | bond | — | cyclopropyl | 2.01* |
| 940 | CR³ | CR⁴ | H | H | H | H | Cl | H | H | Cl | H | bond | — | cyclopropyl | 2.6* |
| 941 | CR³ | CR⁴ | H | H | Cl | Cl | H | H | H | Cl | H | bond | — | cyclopropyl | 3.46* |
| 942 | CR³ | CR⁴ | H | H | Cl | Br | H | H | H | Cl | H | bond | — | cyclopropyl | 3.55* |
| 943 | CR³ | CR⁴ | H | H | H | H | di- | H | H | Cl | H | bond | — | cyclopropyl | 2.36* |
| | | | | | | | fluoro- | | | | | | | | |
| | | | | | | | meth- | | | | | | | | |
| | | | | | | | oxy | | | | | | | | |
| 944 | CR³ | CR⁴ | H | H | H | H | (tri- | H | H | Cl | H | bond | — | cyclopropyl | 3.72* |
| | | | | | | | fluoro- | | | | | | | | |
| | | | | | | | meth- | | | | | | | | |
| | | | | | | | yl-thio | | | | | | | | |
| 945 | CR³ | CR⁴ | H | H | F | H | Me | H | H | Cl | H | bond | — | cyclopropyl | 1.68* |
| 946 | CR³ | CR⁴ | H | H | H | F | F | H | H | Cl | H | bond | — | cyclopropyl | 2.53* |
| 947 | CR³ | CR⁴ | H | H | Cl | H | F | H | H | Cl | H | bond | — | cyclopropyl | 3.03* |
| 948 | CR³ | CR⁴ | F | H | H | F | H | H | H | Cl | H | bond | — | cyclopropyl | 2.91* |
| 949 | CR³ | CR⁴ | H | H | Cl | SO₂NH₂ | H | H | H | Cl | H | bond | — | cyclopropyl | 1.66* |
| 950 | CR³ | CR⁴ | H | H | H | CF₃ | H | H | H | Cl | H | bond | — | cyclopropyl | 3.03* |
| 951 | CR³ | CR⁴ | H | H | Et | H | H | H | H | Cl | H | bond | — | cyclopropyl | 2.13* |
| 952 | CR³ | CR⁴ | H | H | cyclohexyl | H | H | H | H | Cl | H | bond | — | cyclopropyl | 3.29* |
| 953 | CR³ | CR⁴ | H | H | propyl | H | H | H | H | Cl | H | bond | — | cyclopropyl | 2.5* |
| 954 | CR³ | CR⁴ | H | H | sec-butyl | H | H | H | H | Cl | H | bond | — | cyclopropyl | 2.77* |
| 955 | CR³ | CR⁴ | H | H | Me | sec-butoxy | H | H | H | Cl | H | bond | — | cyclopropyl | 2.99* |
| 956 | CR³ | CR⁴ | H | H | NHCOMe | H | H | H | H | CF₃ | H | bond | — | cyclopropyl | 1.64** |
| 957 | CR³ | CR⁴ | H | H | H | ethylpulphinyl | H | H | H | Br | H | bond | — | cyclopropyl | 2.03* |
| 958 | CR³ | CR⁴ | H | H | H | ethylsulphinyl | H | H | H | Br | H | bond | — | cyclopropyl | 1.59* |
| 959 | CR³ | CR⁴ | H | H | H | 3-oxobutyl | H | H | H | CF₃ | H | bond | — | cyclopropyl | 2.68* |
| 960 | CR³ | CR⁴ | H | H | H | dimethylcarbamoyl | H | H | H | Cl | H | bond | — | cyclopropyl | 1.33* |
| 961 | CR³ | CR⁴ | H | H | H | 2,2,2-trifluoro-1- | H | H | H | Cl | H | bond | — | cyclopropyl | 1.79* |
| | | | | | | hydroxyethyl | | | | | | | | | |
| 962 | CR³ | CR⁴ | H | H | H | 1-methoxyethyl | H | H | H | CF₃ | H | methylene | — | cyclopropyl | 3.29* |
| 963 | CR³ | CR⁴ | H | H | H | H | H | H | H | Cl | Me | methylene | — | cyclopropyl | 2.72** |
| 964 | CR³ | CR⁴ | H | H | H | 2-oxopyrrolidin- | H | H | H | Cl | Me | methylene | — | cyclopropyl | 2.21** |
| | | | | | | 1-yl | | | | | | | | | |
| 965 | CR³ | CR⁴ | H | H | H | methoxymethyl | H | H | H | Cl | Me | methylene | — | cyclopropyl | 2.71** |
| 966 | CR³ | CR⁴ | H | H | 2-oxopyrrolidin- | H | H | H | H | Cl | Me | methylene | — | cyclopropyl | 2.09** |
| | | | | | 1-yl | | | | | | | | | | |
| 967 | CR³ | CR⁴ | H | H | H | 3-oxobutyl | H | H | H | Br | H | bond | — | cyclopropyl | 1.83* |
| 968 | CR³ | CR⁴ | H | H | H | SMe | H | H | H | Cl | Me | methylene | — | cyclopropyl | 3.6** |
| 969 | CR³ | CR⁴ | H | H | H | Me | H | Me | H | Cl | H | bond | — | cyclopropyl | 1.73* |
| 970 | CR³ | CR⁴ | H | H | (methoxycarbonyl)- | H | H | H | H | Cl | Me | methylene | — | cyclopropyl | 1.99** |
| | | | | | amino | | | | | | | | | | |
| 971 | CR³ | CR⁴ | H | H | methoxycarbonyl | H | H | H | H | CF₃ | H | bond | — | cyclopropyl | 3.21* |
| 972 | CR³ | CR⁴ | H | H | H | OMe | H | Me | H | Cl | H | bond | — | cyclopropyl | 1.55* |
| 973 | CR³ | CR⁴ | H | H | H | isopropyl | H | H | H | CF₃ | H | bond | — | cyclopropyl | 4.14* |
| 974 | CR³ | CR⁴ | H | H | H | H | H | H | CF₃ | Cl | H | bond | — | cyclopropyl | 4.24** |
| 975 | CR³ | CR⁴ | H | H | H | 2-oxopyrrolidin- | H | Me | H | CF₃ | H | bond | — | cyclopropyl | 2.14* |
| | | | | | | 1-yl | | | | | | | | | |
| 976 | CR³ | CR⁴ | H | H | H | cyanomethyl | H | H | H | CF₃ | H | bond | — | cyclopropyl | 2.56* |
| 977 | CR³ | CR⁴ | H | H | (ethoxycarbonyl)- | H | H | H | H | Cl | H | bond | — | cyclopropyl | 1.63* |
| | | | | | amino | | | | | | | | | | |
| 978 | CR³ | CR⁴ | H | H | formyl(methyl)- | H | H | H | H | Cl | H | bond | — | cyclopropyl | 1.44* |
| | | | | | amino | | | | | | | | | | |
| 979 | CR³ | CR⁴ | H | H | —OCONH— | | H | H | H | Cl | H | bond | | cyclopropyl | 1.37* |
| 980 | CR³ | CR⁴ | H | —N(COCH₃)CH₂CH₂— | | H | H | H | H | Cl | H | bond | | cyclopropyl | 1.47* |
| 981 | CR³ | CR⁴ | H | H | —NHCOO— | | H | H | H | Cl | H | bond | | cyclopropyl | 1.37* |
| 982 | CR³ | CR⁴ | H | H | Me | NHCOMe | H | H | H | Cl | H | bond | — | cyclopropyl | 1.34* |
| 983 | CR³ | CR⁴ | H | H | H | propylthio | H | H | H | Cl | Me | bond | — | cyclopropyl | 4.88* |
| 984 | CR³ | CR⁴ | H | H | diethylcarbamoyl | H | H | H | H | Cl | H | bond | — | cyclopropyl | 1.84* |
| 985 | CR³ | CR⁴ | H | H | H | 2-oxopyrrolidin- | H | H | COMe | Cl | H | bond | — | cyclopropyl | 1.75** |
| | | | | | | 1-yl | | | | | | | | | |
| 986 | CR³ | CR⁴ | H | H | H | (ethylthio)methyl | H | H | H | Cl | H | bond | — | cyclopropyl | 2.55* |
| 987 | CR³ | CR⁴ | H | H | H | 1-(ethyl- | H | H | H | CF₃ | H | bond | — | cyclopropyl | 4.19* |
| | | | | | | sulphanyl)ethyl | | | | | | | | | |
| 988 | CR³ | CR⁴ | H | H | H | 1-(ethyl- | H | H | H | CF₃ | H | methylene | — | cyclopropyl | 4.37* |
| | | | | | | sulphanyl)ethyl | | | | | | | | | |
| 989 | CR³ | CR⁴ | H | H | isopropoxy | H | H | H | H | Cl | H | bond | — | cyclopropyl | 1.97* |
| 990 | CR³ | CR⁴ | H | H | Cl | ethoxycarbonyl | H | H | H | Cl | H | bond | — | cyclopropyl | 2.84* |
| 991 | CR³ | CR⁴ | H | H | Me | butoxy | H | H | H | Cl | H | bond | — | cyclopropyl | 3.13* |
| 992 | CR³ | CR⁴ | H | H | isopropoxy | isopropoxy | H | H | H | Cl | H | bond | — | cyclopropyl | 2.42* |
| 993 | CR³ | CR⁴ | H | H | hydroxyl | methoxycarbonyl | H | H | H | Cl | H | bond | — | cyclopropyl | 1.81* |
| 994 | CR³ | CR⁴ | H | H | Me | isobutoxy | H | H | H | Cl | H | bond | — | cyclopropyl | 3.17* |
| 995 | CR³ | CR⁴ | H | H | —N═CH—S— | | H | H | H | Cl | H | bond | — | cyclopropyl | 1.66* |
| 996 | CR³ | CR⁴ | H | H | H | 2-oxopyrrolidin- | H | H | CF₃ | Cl | H | bond | — | cyclopropyl | 3.43* |
| | | | | | | 1-yl | | | | | | | | | |
| 997 | CR³ | CR⁴ | H | H | H | (2-oxopyrrolidin- | H | H | H | Cl | Me | bond | — | cyclopropyl | 2.15* |
| | | | | | | 1-yl)methyl | | | | | | | | | |
| 998 | CR³ | CR⁴ | H | Br | H | CF₃ | H | H | H | Cl | H | bond | — | cyclopropyl | 4.6* |
| 999 | CR³ | CR⁴ | H | H | H | ethoxymethyl | H | H | H | Cl | H | bond | — | cyclopropyl | 1.99* |
| 1000 | CR³ | CR⁴ | H | H | propane-1,3-diyl | | H | H | H | Cl | H | bond | — | cyclopropyl | 2.11* |
| 1001 | CR³ | CR⁴ | H | methoxy | H | methoxycarbonyl | H | H | H | Cl | H | bond | — | cyclopropyl | 2.46* |
| | | | | carbonyl | | | | | | | | | | | |
| 1002 | CR³ | CR⁴ | H | H | H | phenyl | H | H | H | Cl | H | bond | — | cyclopropyl | 2.77* |
| 1003 | CR³ | CR⁴ | H | H | Me | isopropoxy | H | H | H | Cl | H | bond | — | cyclopropyl | 2.57* |
| 1004 | CR³ | CR⁴ | H | H | OMe | methoxycarbonyl | H | H | H | Cl | H | bond | — | cyclopropyl | 1.58* |
| 1005 | CR³ | CR⁴ | H | H | H | 1H-pyrrol-1-yl | H | H | H | Cl | H | bond | — | cyclopropyl | 2.67* |
| 1006 | CR³ | CR⁴ | H | H | H | isopropylcarbonyl | H | H | H | Cl | H | bond | — | cyclopropyl | 2.63* |
| 1007 | CR³ | CR⁴ | H | H | H | 1,3-thiazol-4-yl | H | H | H | Cl | H | bond | — | cyclopropyl | 1.9* |
| 1008 | CR³ | CR⁴ | H | H | H | H | H | H | H | Cl | H | bond | — | 2-methyl- | 2** |
| | | | | | | | | | | | | | | cyclopropyl | |
| 1009 | CR³ | CR⁴ | H | H | H | 2-oxoimidazolidin- | H | H | H | Cl | H | bond | — | cyclopropyl | 1.23* |
| | | | | | | 1-yl | | | | | | | | | |
| 1010 | CR³ | CR⁴ | H | H | H | ethoxymethyl | H | H | H | CF₃ | H | bond | — | cyclopropyl | 3.23* |
| 1011 | CR³ | CR⁴ | H | methoxy | H | methoxymethyl | H | H | H | CF₃ | H | bond | — | cyclopropyl | 2.79* |
| | | | | methyl | | | | | | | | | | | |
| 1012 | CR³ | CR⁴ | H | methoxy | H | methoxymethyl | H | H | H | Cl | H | bond | — | cyclopropyl | 1.76* |
| | | | | methyl | | | | | | | | | | | |
| 1013 | CR³ | CR⁴ | H | H | phenylthio | H | H | H | H | Cl | H | bond | — | cyclopropyl | 3.69* |
| 1014 | CR³ | CR⁴ | H | H | H | H | H | H | H | Cl | H | bond | — | 2-phenyl- | 2.77** |
| | | | | | | | | | | | | | | cyclopropyl | |
| 1015 | CR³ | CR⁴ | H | H | Cl | 1-methoxyethyl | H | H | H | Cl | H | bond | — | cyclopropyl | 3.1* |
| 1016 | CR³ | CR⁴ | H | H | H | 1-ethoxyethyl | H | H | H | Cl | H | bond | — | cyclopropyl | 2.3* |
| 1017 | CR³ | CR⁴ | H | CN | H | CF₃ | H | H | H | Cl | H | bond | — | cyclopropyl | 3.69** |
| 1018 | CR³ | CR⁴ | H | H | Me | 2-oxo-1,3- | H | H | H | Cl | H | bond | — | cyclopropyl | 1.37* |
| | | | | | | oxazolidin-3-yl | | | | | | | | | |
| 1019 | CR³ | CR⁴ | H | H | H | 2-(dimethylamino)- | H | H | H | Cl | H | bond | — | cyclopropyl | 1.29* |
| | | | | | | 2-oxoethyl | | | | | | | | | |
| 1020 | CR³ | CR⁴ | H | H | H | 2-oxopiperidin- | H | H | H | Cl | H | bond | — | cyclopropyl | 1.53** |
| | | | | | | 1-yl | | | | | | | | | |
| 1021 | CR³ | CR⁴ | H | H | acetamidosulphonyl | H | H | H | H | Cl | H | bond | — | cyclopropyl | 1.54** |
| 1022 | CR³ | CR⁴ | H | H | H | SMe | H | H | CF₃ | Cl | H | bond | — | cyclopropyl | 4.64** |
| 1023 | CR³ | CR⁴ | H | H | H | bromomethyl | H | H | H | Cl | H | bond | — | cyclopropyl | 2.31** |
| 1024 | CR³ | CR⁴ | H | H | H | allyloxy | H | H | H | CF₃ | H | bond | — | cyclopropyl | 3.54** |
| 1025 | CR³ | CR⁴ | H | H | H | H | H | H | H | Cl | 4- | bond | — | cyclopropyl | 4.15** |
| | | | | | | | | | | | meth- | | | | |
| | | | | | | | | | | | oxy- | | | | |
| | | | | | | | | | | | benzyl | | | | |
| 1026 | CR³ | CR⁴ | H | H | H | SMe | H | H | H | Cl | 4- | bond | — | cyclopropyl | 4.78** |
| | | | | | | | | | | | meth- | | | | |
| | | | | | | | | | | | oxy- | | | | |
| | | | | | | | | | | | benzyl | | | | |
| 1027 | CR³ | CR⁴ | H | H | H | H | H | H | H | Cl | H | bond | — | 2,2-dimethyl- | 2.37*; |
| | | | | | | | | | | | | | | cyclopropyl | 2.37** |
| 1028 | CR³ | CR⁴ | H | H | H | methoxymethyl | H | H | H | Cl | H | bond | — | 2,2-dimethyl- | 2.36*; |
| | | | | | | | | | | | | | | cyclopropyl | 2.37** |
| 1029 | CR³ | CR⁴ | H | H | H | SMe | H | H | H | Cl | H | bond | — | 2,2-dimethyl- | 3.07*; |
| | | | | | | | | | | | | | | cyclopropyl | 3.07** |
| 1030 | CR³ | CR⁴ | H | H | dimethylcarbamoyl | H | H | H | H | Cl | H | bond | — | 2,2-dimethyl- | 1.86*; |
| | | | | | | | | | | | | | | cyclopropyl | 1.86** |
| 1031 | CR³ | CR⁴ | H | H | H | ethylcarbamoyl | H | H | H | CF₃ | H | bond | — | cyclopropyl | 2.07* |
| 1032 | CR³ | CR⁴ | H | H | 1-ethylpropyl | H | H | H | H | CF₃ | H | bond | — | cyclopropyl | 4.92* |
| 1033 | CR³ | CR⁴ | H | H | 1-ethylpropyl | H | H | H | H | Cl | H | bond | — | cyclopropyl | 3.41* |
| 1034 | CR³ | CR⁴ | H | H | H | Et | H | H | H | CF₃ | H | bond | — | cyclopropyl | 3.67* |
| 1035 | CR³ | CR⁴ | H | H | H | 2-oxo-1,3- | H | H | H | Cl | H | bond | — | 2,2-dimethyl- | 1.93*; |
| | | | | | | oxazolidin-3-yl | | | | | | | | cyclopropyl | 1.93** |
| 1036 | CR³ | CR⁴ | H | H | H | 1H-imidazol-1-yl | CF₃ | H | H | Cl | H | bond | — | cyclopropyl | 1.41* |
| 1037 | CR³ | CR⁴ | H | H | H | 1-ethoxyethyl | Br | H | H | Cl | H | bond | — | cyclopropyl | 2.49* |
| 1038 | CR³ | CR⁴ | —CH═CH—CH═N— | | H | H | H | H | H | Cl | H | bond | — | cyclopropyl | 1.01* |
| 1039 | CR³ | CR⁴ | H | H | H | 2-methoxyethyl | H | H | H | CF₃ | H | bond | — | cyclopropyl | 2.88** |
| 1040 | CR³ | CR⁴ | H | H | H | 2-morpholin-4- | H | H | H | C | H | bond | — | cyclopropyl | 0.84* |
| | | | | | | ylethoxy | | | | | | | | | |
| 1041 | CR³ | CR⁴ | H | H | H | 2-methoxyethoxy | H | H | H | CF₃ | H | bond | — | cyclopropyl | 2.78* |
| 1042 | CR³ | CR⁴ | H | H | H | 2-morpholin-4- | H | H | H | Cl | H | bond | — | cyclopropyl | 1.42* |
| | | | | | | yl-2-oxoethyl | | | | | | | | | |
| 1043 | CR³ | CR⁴ | H | H | H | pyrrolidin-1- | H | H | H | Cl | H | bond | — | cyclopropyl | 0.92** |
| | | | | | | ylmethyl | | | | | | | | | |
| 1044 | CR³ | CR⁴ | H | H | H | methoxymethyl | H | H | H | Cl | 4- | bond | — | cyclopropyl | 4.07** |
| | | | | | | | | | | | meth- | | | | |
| | | | | | | | | | | | oxy- | | | | |
| | | | | | | | | | | | benzyl | | | | |
| 1045 | CR³ | CR⁴ | H | H | H | 2-oxopyrrolidin- | H | H | H | Cl | 4- | bond | — | cyclopropyl | 3.37** |
| | | | | | | 1-yl | | | | | meth- | | | | |
| | | | | | | | | | | | oxy- | | | | |
| | | | | | | | | | | | benzyl | | | | |
| 1046 | CR³ | CR⁴ | H | H | H | 1H-imidazol-1- | H | H | H | Cl | H | bond | — | cyclopropyl | 0.83** |
| | | | | | | ylmethyl | | | | | | | | | |
| 1047 | CR³ | CR⁴ | H | H | F | hydroxymethyl | H | H | H | Cl | H | bond | — | cyclopropyl | 1.25* |
| 1048 | CR³ | CR⁴ | H | H | pentyl | H | H | H | H | Cl | H | bond | — | cyclopropyl | 3.55* |
| 1049 | CR³ | CR⁴ | H | H | H | diethylcarbamoyl | H | H | H | CF₃ | H | bond | — | cyclopropyl | 2.71* |
| 1050 | CR³ | CR⁴ | H | H | OMe | methoxymethyl | H | H | H | Cl | H | bond | — | cyclopropyl | 1.55* |
| 1051 | CR³ | CR⁴ | H | H | H | Me | H | H | H | Cl | H | methylene | — | cyclopropyl | 2.09* |
| 1052 | CR³ | CR⁴ | H | H | H | SF5 | H | H | H | Cl | H | bond | — | cyclopropyl | 3.7** |
| 1053 | CR³ | CR⁴ | H | H | F | difluoromethoxy | H | H | H | Cl | H | bond | — | cyclopropyl | 2.62* |
| 1054 | CR³ | CR⁴ | H | H | H | butylthio | H | H | H | Cl | H | bond | — | cyclopropyl | 3.78* |
| 1055 | CR³ | CR⁴ | H | H | H | 2-oxo-1,3- | H | H | H | Cl | Me | bond | — | cyclopropyl | 2.13* |
| | | | | | | oxazolidin-3-yl | | | | | | | | | |
| 1056 | CR³ | CR⁴ | H | H | H | 2-oxomorpholin- | H | H | H | Cl | Me | bond | — | cyclopropyl | 1.86* |
| | | | | | | 4-yl | | | | | | | | | |
| 1057 | CR³ | CR⁴ | H | H | H | Me | H | H | H | Cl | Me | bond | — | cyclopropyl | 3.08* |
| 1058 | CR³ | CR⁴ | H | H | H | Et | H | H | H | Cl | Me | bond | — | cyclopropyl | 3.65* |
| 1059 | CR³ | CR⁴ | H | H | H | (4-methylpiperazin- | H | H | H | Cl | H | bond | — | cyclopropyl | 0.59* |
| | | | | | | 1-yl)carbonyl | | | | | | | | | |
| 1060 | CR³ | CR⁴ | H | H | H | ethyl(methyl)- | H | H | H | Cl | H | bond | — | cyclopropyl | 1.56* |
| | | | | | | carbamoyl | | | | | | | | | |
| 1061 | CR³ | CR⁴ | H | H | H | [2-(methoxy- | H | H | H | Cl | H | bond | — | cyclopropyl | 1.77* |
| | | | | | | methyl)pyrrolidin- | | | | | | | | | |
| | | | | | | 1-yl]carbonyl | | | | | | | | | |
| 1062 | CR³ | CR⁴ | H | H | H | (2-methylpyrrolidin- | H | H | H | Cl | H | bond | — | cyclopropyl | 1.86* |
| | | | | | | 1-yl)carbonyl | | | | | | | | | |
| 1063 | CR³ | CR⁴ | H | H | H | azetidin-1-ylcarbonyl | H | H | H | Cl | H | bond | — | cyclopropyl | 1.43* |
| 1064 | CR³ | CR⁴ | H | H | H | (2-methoxy-ethyl)- | H | H | H | Cl | H | bond | — | cyclopropyl | 1.46* |
| | | | | | | (methyl)carbamoyl) | | | | | | | | | |
| 1065 | CR³ | CR⁴ | H | H | H | piperazin-1- | H | H | H | Cl | H | bond | — | cyclopropyl | 0.53* |
| | | | | | | ylcarbonyl | | | | | | | | | |
| 1066 | CR³ | CR⁴ | H | H | H | cyclopentyl | H | H | H | Cl | H | bond | — | cyclopropyl | 3.18* |
| 1067 | CR³ | CR⁴ | H | H | H | thiomorpholin-4- | H | H | H | Cl | H | bond | — | cyclopropyl | 1.8* |
| | | | | | | ylcarbonyl | | | | | | | | | |
| 1068 | CR³ | CR⁴ | H | H | H | propylsulphonyl | H | H | H | CF₃ | H | methylene | — | cyclopropyl | 3.25* |
| 1069 | CR3 | CR4 | H | H | H | H | H | H | H | Br | H | bond | — | cyclopropyl | 2.65* |

^{a}The LogP values were determined in accordance with EEC Directive 79/831 Annex V.A8 by HPLC (High Performance Liquid Chromatography) on reversed-phase columns (C 18), using the methods below:
*temperature: 40° C.; mobile phase: 0.1% aqueous formic acid and acetonitrile; linear gradient from 10% acetonitrile to 95% acetonitrile.
**temperature: 43° C.; mobile phase: 0.1% aqueous phosphoric acid and acetonitrile; linear gradient from 10% acetonitrile to 95% acetonitrile.

Calibration was in each case carried out using unbranched alkan-2-ons (3 to 16 carbon atoms) with known logP values (determination of the logP values via the retention times by linear interpolation between two known alkanones).

The lambda-max values were in each case determined in the maxima of the chromatographic signals of the UV spectra between 190 nm and and 400 nm.

### Use Examples

### Example A

Venturia test (apple)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention of the formulae below show, at an active compound concentration of 100 ppm, an efficacy of 70% or more.

Example: 2, 10, 15, 24, 28, 29, 30, 33, 34, 37, 43, 45, 48, 51, 59, 60, 61, 65, 67, 68, 69, 70, 71, 72, 73, 74, 75, 76, 77, 78, 80, 86

### Example B

Botrytis test (bean)/protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

2 days after the inoculation, the size of the infected areas on the leaves is evaluated. 0% means an efficacy which corresponds to that of the control, whereas an efficacy of 100% means that no infection is observed.

In this test, the compounds according to the invention of the formulae below show, at an active compound concentration of 250 ppm, an efficacy of 70% or more.

Example: 2, 29, 30, 60, 63, 64, 65, 67, 68, 69, 70, 71, 72, 75, 80

### Example C

Alternaria test (tomato)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifer, and the concentrate is diluted with water to the desired concentration.

To test for protective activity, young tomato plants are sprayed with the active compound preparation at the stated application rate. 1 day after the treatment, the plants are inoculated with a spore suspension of Alternaria solani and then remain at 100% relative humidity and 20° C. for 24 h. The plants then remain at 96% relative atmospheric humidity and a temperature of 20° C.

Evaluation is carried out 7 days after the inoculation. 0% means an efficacy which corresponds to that of the control, whereas an efficacy of 100% means that no infection is observed.

In this test, the compounds according to the invention of the formulae below show, at an active compound concentration of 500 ppm, an efficacy of 70% or more.

Example: 2, 3, 5, 12, 19, 22, 28, 35, 45, 47, 50, 51, 56, 59, 60, 61, 63, 64, 65, 67, 68, 69, 70, 71, 72, 73, 75, 77, 84, 91, 95, 97, 99, 100, 101, 102, 107, 110

### Example D

Sphaerotheca test (cucumber)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. 1 day after the treatment, the plants are inoculated with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the compounds according to the invention of the formulae below show, at an active compound concentration of 500 ppm, an efficacy of 70% or more.

Example: 2, 15, 16, 18, 24, 28, 29, 30, 33, 58, 59, 74, 76, 80, 82, 88, 89, 93, 98, 101, 104

### Example E

Puccinia test (wheat)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Puccinia recondita. The plants remain in an incubation cabin at 20° C. and 100% relative atmospheric humidity for 48 hours. The plants are then placed in a greenhouse at a temperature of about 20° C. and a relative atmospheric humidity of 80% to promote the development of rust pustules.

Evaluation is carried out 10 days after the inoculation. 0% means an efficacy which corresponds to that of the control, whereas an efficacy of 100% means that no infection is observed.

In this test, the compounds according to the invention of the formulae below show, at an active compound concentration of 1000 ppm, an efficacy of 70% or more.

Example: 2, 4, 6, 12, 16, 29, 30, 33, 34, 43, 45, 48, 54, 58, 60, 61, 63, 67, 74, 76, 80, 86, 88, 91

1. A fungicide comprising a diaminopyrimidine compound of formula (I) 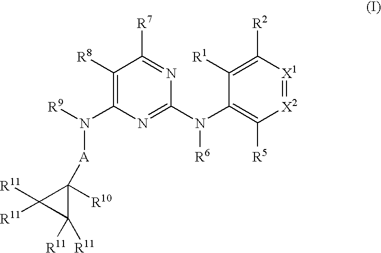
where the symbols have the following meanings:
X¹ represents nitrogen or CR³,
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or
unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃, CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3-to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted
or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

2. A fungicide of claim 1 in which
X¹ represents nitrogen or CR³,
X² represents nitrogen or CR⁴,
A represents a direct bond, methylene or —CH(CH₃)—,
R¹ to R⁵ independently of one another represent hydrogen, fluorine, chlorine, bromine, iodine, cyano, hydroxyl, nitro, OMe, OEt, OPr, OisoPr, OBu, OsecBu, OisoBu, OtertBu, O—(CH₂)₂OCH₃, O—(CH₂)₃OCH₃, O-cyclopentyl, OCF₃, OCF₂H, OCF₂CF₃, OCF₂CF₂H, OCH₂CH₂N(C₂H₅)₂, OCH(CH₃)CH₂OCH₃, SH, SMe, SPh, SEt, SPr, SisoPr, SBu, SsecBu, SisoBu, StBu, S Spentyl, Ssecpentyl, Sneopentyl, SOctyl, CF₃, SCF₂H, SOMe, SO₂Me, SO₂CF₃, SO₂Et, SO₂Pr, SO₂CH₂CH═CH₂, SO₂CH₂CN, SO₂CH₂C≡CH, SONHMe, SONMe₂, SO₂NHMe, SO₂NMe₂, O₂NH₂, SO₂NHAc, SO₂NHPh, SO₂NHnBu, SO₂NHtBu, SO₂NEt₂, SO₂NHEt, SO₂NPr₂, COOH, COMe, SO₂NH(CH₂)₃NMe₂, SO₂NHCH₂CH═CH₂, COEt, COPr, COisoPr, COBu, COsecBu, COisoBu, COtertBu, COCHF₂, COCF₃, NHCOOMe, NHCOOisoPr, NHCOOtertBu, NHCOOnBu NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCOCH₂OCH₃, NHCO(CH₂)₂OCH₃, N(CH₃)COCH₃, N(C₂H₅)COCH₃, N(CH₃)COC(CH₃)₃, N(C₂H₅)COOCH₃, NHCHO, N(CH₃)CHO, NMe₂, NEt₂, NHMe, NH₂, NHtertBu, NHEt, NHPr, NHisoPr, NHBu, NHisoBu, NHsecBu, cyclopropylamino, NHCH(CH₃)CH₂OCH₃, NCH₃COCH₃, acetyl(cyclopropyl)amino, [(1-methylcyclopropyl)carbonyl]amino, morpholin-1-yl, morpholin-4-ylmethyl, NHSO₂CH₃NHSOMe, NHSO₂Me, NHSOCF₃, NHSO₂CF₃, OCONHCH₃, OCONHEt, OCONHPr, OCONHisoPr, OCON(CH₃)₂, OCON(Et)₂, OCOMe, OCOEt, OCOPr, OCOisoPr, OCOBu, OCOsecBu, OCOisoBu, OCOtertBu, OSO₂N(CH₃)₂, CONHEt, CONEt₂, CONHCH₃, CON(CH₃)₂, CONHPr, CONHisoPr, CONHPh, COCH₂CN, CONPr₂, CONHBu, CONHtertBu, CONHCH₂CH═CH₂, CONHCH(CH₃)CH₂OCH₃, CONH(CH₂)₂CH₃, COOH, CO₂CH₃, CO₂Et, CO₂Pr, CO₂isoPr, CO₂(CH₂)₂OCH₃, COCH₂N(CH₃)₂, CH₂SO₂NHMe, CH₂SO₂NHisoPr, CH₂SO₂NHPr, CH₂SO₂NHtertBu, CH₂COtertBu, CH₂COCH₃, CH₂COOEt, C(CH₃)₂OCH₃, CHCF₃OCH₃, CHCF₃OC₂H₅, CHCF₃OH, CH₂C(CH₃)₂OCH₃, CHCHF₂OH, CH₂OH, CH₂NHCOOBn, CH₂NHCOOtertBu, CH₂SO₂CH₃, CH₂NH(CH₂)₂OCH₃, CH₂OMe, (CH₂)₂Ome, (CH₂)₃OMe, CH₂SMe, (CH₂)₂SMe, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, CH₂NHCOOMe, CH₂NHCOOtertBu, CH₂NHCOOEt, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, cyclopentyl, CF₃, CF₂H, CCl₃, C₂F₅, 4-(tert-butoxycarbonyl)piperazin-1-yl, morpholin-4-ylsulphonyl, morpholin-4-ylcarbonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl -2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxo-morpholin-4-yl, 2-oxoazetidin-1-yl, 2,5-dioxo-2,5-dihydro-1H-pyrrol-1-yl,
if in each case two adjacent radicals R², R³ or R⁴, if appropriate via R¹² or R¹³, form a cycle, the following subunit of formula (I): 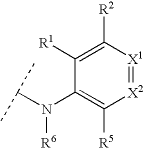
may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethyl-sulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino,
R⁶ represents hydrogen, Me, formyl, COMe, COOMe, COOEt, COOtertBu, COOBn, COCF₃, benzyl,
R⁷ represents hydrogen, methyl, CF₃,
R⁸ represents fluorine, chlorine, bromine, iodine, cyano, methyl, CF₃, CCl₃, CFH₂, CF₂H,
R⁹ represents hydrogen, Me,
R¹⁰ represents hydrogen, OEt, COOEt, 2-chlorophenyl,
R¹¹ represents identical or different hydrogen, fluorine, chlorine,
And/or an agrochemically active salt thereof.

3. Composition for controlling unwanted microorganisms, comprising a fungicide comprising at least one diaminopyrimidine and/or salt of formula (I) according to claim 1, and an extender and/or surfactant.

4. Composition according to claim 3, comprising at least one further agrochemically active compound.

5. Method for controlling unwanted microorganisms, comprising applying a fungicide comprising a compound and/or salt of formula (I) according to claim 1 to the unwanted microorganisms and/or a habitat thereof.

6. Process for preparing compositions for controlling unwanted microorganisms, comprising mixing a fungicide according to claim 1 with an extender and/or surfactant.

7. Compounds of formula (Ia) 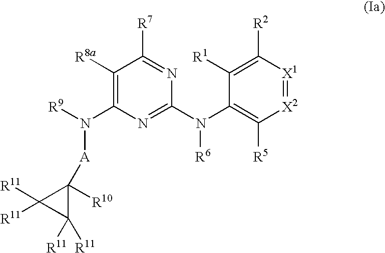
in which the symbols have the following meanings:
R^{8a} represents chlorine, iodine, CFH₂, CF₂H or CCl₃ and
X¹ represents nitrogen or CR³,
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulfinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

8. Compounds of formula (Ib) 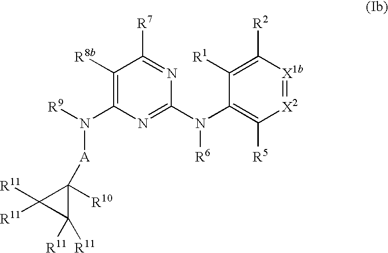
in which the symbols have the following meanings:
X^{1b} represents nitrogen or C—R^{3b}, and
R^{3b} represents hydrogen, halogen, cyano, hydroxyl, nitro, an unsubstituted or substituted, saturated, partially unsaturated or aromatic, heterocyclic or carbocyclic five-, six- or seven-membered ring, OR¹², SR¹², SOR¹², SO₂CF₃, SO₂Bu, SO₂secBu, SO₂isoBu, SO₂-tertBu, SO₂-pentyl, SO₂neopentyl, SO₂CH₂CH═CH₂, SO₂CH₂CN, SO₂CH₂C≡CH, SON(R¹²)₂, SO₂NHMe, SO₂NMe₂, SO₂NH₂, SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe₂, SO₂NH(CH₂)₃NEt₂, SO₂NHCH₂CH═CH₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8
where additionally or independently thereof in each case two adjacent radicals R², R^{3b} or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another; and
R^{8b} represents cyano and
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSOR¹², (CH₂)ₘSO₂R¹², (CH₂)ₘSON(R¹²)₂, (CH²)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—CH≡CH—R^{1-A}, —CH₂CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₈-trialkylsilyl, unsubstituted or substituted aryl, unsubstituted or substituted cyclopropyl,
R₁₂ represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

9. Compounds of formula (Ic) 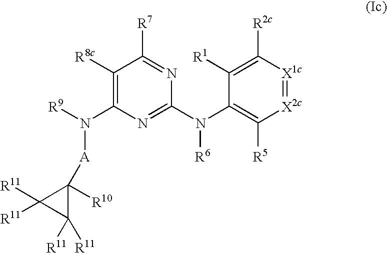
in which the symbols have the following meanings:
X^{1c} represents nitrogen or C—R^{3c},
R^{2c} and R^{4c} independently of one another represent hydrogen, halogen, cyano, hydroxyl, nitro, an unsubstituted or substituted, saturated, partially unsaturated or aromatic, heterocyclic or carbocyclic five-, six- or seven-membered ring, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF₃, OCF₂CF₂H, OCH₂CF₂H, OCH₂CF₃, OCH₂CH₂N(C₂H₅)₂, OCH₂CH₂N(CH₃)₂, OCH(CH₃)CH₂OCH₃, SR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8
where additionally or independently thereof in each case two adjacent radicals R^{2c}, R^{3c} or R^{4c}, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another;
R^{3c} represents hydrogen, fluorine, chlorine, bromine, iodine, cyano, hydroxyl, nitro, OMe, OEt, OPr, OisoPr, OBu, OsecBu, OisoBu, OtertBu, Opentyl, Oneopentyl O—(CH₂)₂OH, O—(CH₂)₂OCH₃, O—(CH₂)₃OH, O—(CH₂)₃OCH₃, O-cyclopentyl, O-cyclopropyl, O-cyclobutyl, O-cyclohexyl, OCF₃, OCF₂H, OCFH₂, OCF₂CF₃, OCF₂CF₂H, OCH₂CF₂H, OCH₂CF₃, OCH₂CH₂N(C₂H₅)₂, OCH₂CH₂N(CH₃)₂, OCH(CH₃)CH₂OCH₃, SH, SMe, SPh, SEt, SPr, SisoPr, SBu, SsecBu, SisoBu, StBu, Spentyl, Ssecpentyl, Sneopentyl, SOctyl, SCF₃, SCF₂H, SCFH₂, SOMe, SO-Et, SO—Pr, SOisoPr, SOBu, SOsecBu, SOisoBu, SO-tBu, SO-pentyl, SOneopentyl, SO₂Me, SO₂CF₃, SO₂Et, SO₂Pr, SO₂isoPr, SO₂Bu, SO₂secBu, SO₂isoBu, SO₂-tBu, SO₂-pentyl, SO₂neopentyl, SO₂CH₂CH═CH₂, SO₂CH₂CN, SO₂CH₂C≡CH, SONHMe, SONMe₂, SONHEt, SONEt₂, SONHPr, SONPr₂, SONH-cyclopropyl, SON(cyclopropyl)₂, SONMe-cyclopropyl, SONHnBu, SONHisoBu, SONHtertBu, SONHpentyl, SONBu₂, SONHCF₃, SON(CF₃)₂, SO₂NHMe, SO₂NMe₂, SO₂NH₂, SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe₂, SO₂NH(CH₂)₃NEt₂, SO₂NHCH₂CH═CH₂, COMe, COEt, COPr, COisoPr, COBu, COsecBu, COisoBu, COtertBu, COcyclopropyl, COCHF₂, COCH₂F, COCF₃, NHCOOMe, NHCOOisoPr, NHCOO-tertBu, NHCOOnBu, NHCOOpentyl, NHCOOcycloproyl, NH(C═S)OMe, NHCOMe, NHCOCF₃, NHCOEt, NHCOPr, NHCOisoPr, NHCOBu, NHCOisoBu, NHCOsecBu, NHCOisoBu, NHCOtertBu, N(Et)COMe, NHCOCH═CH₂, NHCOPh, NHCOC(CH₃)₂CH₂F, NHCOC(CH₃)₂CH₂Cl, NHCO(C═CH₂)CH₃, NHCON(CH₃)₂, NHCO(CH₂)₂OH, NHCOCH₂OCH₃, NHCO(CH₂)₂OCH₃, NHCO(CH₂)₃OH, NHCO(CH₂)₃OCH₃, NHCO(CH₂)₃OEt, N(C₂H₅)COCH₃, N(CH₃)COEt, N(C₂H₅)COEt, N(CH₃)COC(CH₃)₃, N(C₂H₅)COOCH₃, N(C₂H₅)COOEt, N(C₂H₅)COOPr, N(C₂H₅)COOBu, N(C₂H₅)COOtertBu, NHCHO, N(CH₃)CHO, N(Et)CHO, NMe₂, NEt₂, NPr₂, NBu₂, NisoPr₂, NisoBu₂, N-sBu₂, N-tBu₂, NHMe, NH₂, NHtertBu, NHsBu, NHEt, NHPr, NHisoPr, NHBu, NHisoBu, NHsecBu, cyclopropylamino, NH(Et)cyclopropyl, NH(Et)cyclopropyl, NHCH(CH₃)CH₂OCH₃, NHCH(CH₃)CH₂OEt, NCH₃COCH₃, NEtCOCH₃, acetyl(cyclopropyl)amino, [(1-methylcyclopropyl)carbonyl]amino, NHSOMe, NHSO₂Me, NHSOEt, NHSO₂Et, NMeSOMe, NMeSO₂Me, NMeSOEt, NMeSO₂Et, NHSOPr, NHSO₂Pr, NMeSOPr, NMeSO₂Pr, NHSOisoPr, NHSO₂isoPr, NMeSOisoPr, NMeSO₂isoPr, NHSOBu, NHSO₂Bu, NMeSOBu, NMeSO₂Bu, NHSOtertBu, NHSO₂tBu, NMeSOtertBu, NMeSO₂tBu, NHSOsBu, NHSO₂sBu, NMeSOsBu, NMeSO₂sBu, NHSOisoBu, NHSO₂isoBu, NMeSOisoBu, NMeSO₂isoBu, NHSOCF₃, NHSO₂CF₃, OCONHCH₃, OCONHEt, OCONHPr, OCONHisoPr, OCON(CH₃)₂, OCON(Et)₂, OCONHBu, OCONHsecBu, OCONHisoBu, OCONHtertBu, OCONMe₂, OCONEt₂, OCONPr₂, OCONisoPr₂, OCONBu₂, OCONsecBu₂, OCONisoBu₂, OCONHtertBu, OCONHcyclopropyl, OCON(Me)Et, OCON(Me)Pr, OCON(Me)isoPr, OCON(Me)Bu, OCON(Me)secBu, OCON(Me)isoBu, OCON(Me)tertBu, OCON(Me)cyclopropyl, OCOMe, OCOEt, OCOPr, OCOisoPr, OCOBu, OCOsecBu, OCOisoBu, OCOtertBu, OSO₂N(CH₃)₂, OSO₂NEt₂, CONHEt, CONEt₂, CONHCH₃, CON(CH₃)₂, CONHPr, CONHisoPr, CONHPh, CON(Me)Pr, CON(Me)isoPr, CON(Me)Ph, COCH₂CN, CONPr₂, CONHBu, CONHsecBu, CONHisoBu, CONHtertBu, CON(Me)Bu, CON(Me)secBu, CON(Me)isoBu, CON(Me)tBu, CONHCH₂CH═CH₂, CONHCH(CH₃)CH₂OH, CONHCH(CH₃)CH₂OCH₃, CONHCH(C₂H₅)CH₂OCH₃, CONH(CH₂)₂OCH₃, CONHCH(CH₃)CH₂OEt, CONHCH(C₂H₅)CH₂OEt, CONH(CH₂)₂OEt, CONH(CH₂)₂OH, CONH(CH₂)₃OCH₃, CONH(CH₂)₃OEt, CONH(CH₂)₃OH, COOH, CO₂CH₃, CO₂Et, CO₂Pr, CO₂isoPr, CO₂Bu, CO₂secBu, CO₂isoBu, CO₂tBu, CO₂(CH₂)₂OH, CO₂(CH₂)₂OCH₃, CO₂(CH₂)₂OEt, COCH₂N(CH₃)₂, CO₂(CH₂)₃OCH₃, COCH₂NEt₂, CO₂(CH₂)₃OEt, CH₂SO₂NHMe, CH₂SO₂NHisoPr, CH₂SO₂NHPr, CH₂SO₂NHtertBu, CH₂SO₂NHEt, CH₂COtertBu, CH₂COCH₃, CH₂COEt, CH₂COOEt, CH₂COOMe, C(CH₃)₂OCH₃, CHCH₃OCH₃, CHCF₃OCH₃, CHCF₃OC₂H₅, C(CH₃)₂OEt, CHCH₃OEt, CHCF₃OEt, C(CH₃)₂OH, CHCH₃OH, CHCF₃OH, CH₂C(CH₃)₂OCH₃, CHCHF₂OCH₃, CH₂C(CH₃)₂OEt, CHCHF₂OEt, CHCHF₂OH, CH₂OH, CH₂NHCOOBn, CH₂NHCOOtertBu, CH₂SO₂CH₃, CH₂NH(CH₂)₂OCH₃, CH₂SO₂Et, CH₂NH(CH₂)₂OEt, (CH₂)₂OH, (CH₂)₃OH, (CH₂)₄OH, CH₂OMe, (CH₂)₂OMe, (CH₂)₃OMe, (CH₂)₄OMe, CH₂OEt, (CH₂)₂OEt, (CH₂)₃OEt, (CH₂)₄OEt, CH₂SH, (CH₂)₂SH, (CH₂)₃SH, (CH₂)₄SH, CH₂SMe, (CH₂)₂SMe, (CH₂)₃SMe, (CH₂)₄SMe, —CH₂SEt, (CH₂)₂SEt, (CH₂)₃SEt, (CH₂)₄SEt, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOisoPr, (CH₂)₂COOisoPr, (CH₂)₃COOisoPr, CH₂COOtertBu, (CH₂)₂COOtertBu, (CH₂)₃COOtertBu, CH₂COO(CH₂)₂OH, CH₂COO(CH₂)₂OCH₃, CH₂COO(CH₂)₃OH, CH₂COO(CH₂)₃OCH₃, CH₂NHCOOMe, CH₂NHCOOtertBu, CH₂NHCOOEt, CH₂NHCOOPr, CH₂NHCOOisoPr, CH₂NHCOOBu, CH₂NHCOOtertBu, CH₂NHCOOsecBu, O—(CH₂)₃OCH₃, O-cyclopentyl, CH₂NHCOOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, 1-methoxycyclopropyl, 1-chlorocyclopropyl, cyclobutyl, cyclopentyl, cyclohexyl, CF₃, CF₂H, CCl₃, C₂F₅, C₃F₇, CF(CF₃)₂, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl -2,5-dioxoimidazolidin-1-yl), (4-isopropyl-2-oxo-1,3-oxazolidin-3-yl), (piperidin-1-ylethyl)amino, 4-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl-, 3-oxo-4-ethyl-5-methyl-2,4-dihydropyrazol-2-yl-, 3-oxo-5-trifluoromethyl-2,4-dihydropyrazol2-yl, 3-oxo-2,3a,4,5,6,7-hexahydroindazol-2-yl, 3-oxo-5-isopropyl-2,4-dihydropyrazol-2-yl-, 3,5-dioxo-4,4-dimethylpyrazolidin-1-yl, 3,5-dioxo-4-ethylpyrazolidin-1-yl, 2,5-dioxopyrrolidin-1-yl-, 3-oxo-4,4-dimethylpyrazolidin-1-yl, 3-oxopyrazolidin-1-yl, 3-oxopyrazolidin-1-yl, (2-oxopyrrolidin-1-yl)methyl-, (2-oxopiperidin-1-yl)methyl-, 2-oxopiperidin-1-yl, 3-oxomorpholin-4-yl, 2-oxoazetidin-1-yl, 2,5-dioxo-2,5-dihydro-1H-pyrrol-1-yl,
R^{8c} represents fluorine and
X¹ represents nitrogen or CR³,
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹⁴, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

10. Compounds of formula (Id) 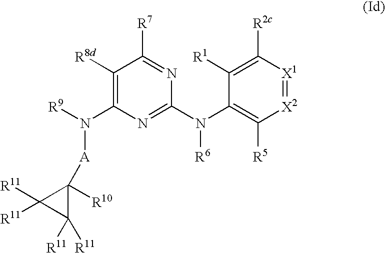
in which the symbols have the following meanings:
X¹ represents nitrogen or CR³,
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³ , NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³ ,NR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆- alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, C₃-C₈-halocycloalkyl)carbonyl having in each case 1 to 9 fluorine, chlorine and/or bromine atoms, or —CH₁—C≡C—R^{1-A}, —CH₂—CH═CH—R^{I-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and with the proviso that
R¹ to R⁵ correspond to the above definitions, except for the following cases: either X¹ represents CR³ and R² and R³, in the following subunit of the general formula (Id): 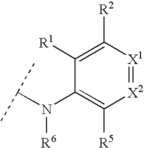
form (2-oxo-2,3-dihydro-1H-indol-5-yl)amino, or X¹ represents CR³ and X² represents CR⁴ and R⁴ and R³, in the above subunit of the general formula (Id), also form (2-oxo-2,3-dihydro-1H-indol-5-yl)amino; and
R^{8d} represents CF₃ and and/or an agrochemically active salt thereof.

11. Compounds of the formula (Ie) 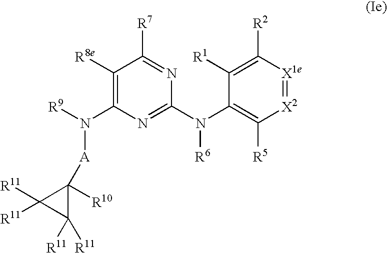
in which the symbols have the following meanings:
X^{1e} represents nitrogen or C—R^{3e},
R^{3e} represents hydrogen, halogen, cyano, hydroxyl, nitro, an unsubstituted or substituted, saturated, partially unsaturated or aromatic, heterocyclic or carbocyclic five-, six- or seven-membered ring, OR¹², SR¹², SOR¹², SO₂R¹², SON(R¹²)₂, SO₂NHMe, SO₂NMe₂, SO₂NHAc, SO₂NMeAc, SO₂N(cyclopropyl)Ac, SO₂NHPh, SO₂NHbenzyl, SO₂NHnBu, SO₂NEt₂, SO₂NHEt, SO₂NPr₂, SO₂NHPr, SO₂NHcyclopropyl, SO₂NHtertBu, SO₂NHCF₃, SO₂N(CF₃)₂, SO₂NH(CH₂)₃NMe₂, SO₂NH(CH₂)₃NEt₂, SO₂NHCH₂CH═CH₂, OSO₂N(R¹²)₂, COEt, COPr, COisoPr, COBu, COsecBu, COisoBu, COtertBu, COcyclopropyl, COCHF2, COCH₂F, COCF₃, NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8
where additionally or independently thereof in each case two adjacent radicals R², R^{3e} or R⁴, if appropriate via R¹² or R¹³, together may form a cycle which, in the following subunit of the general formula (Ie): 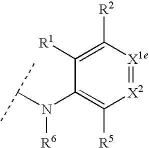
may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1, 4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, 2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1H-indol-5-yl)amino, 2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino;
R^{8e} represents Br and
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R_{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

12. Compounds of formula (If) 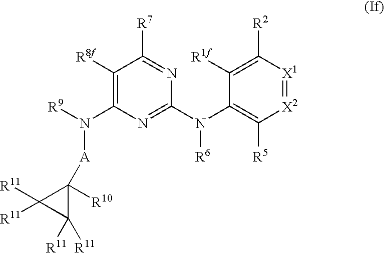
in which the symbols have the following meanings:
R^{1f} represents hydrogen, fluorine, bromine, iodine, cyano, hydroxyl, nitro, an unsubstituted or substituted, saturated, partially unsaturated or aromatic, heterocyclic or carbocyclic five-, six- or seven-membered ring, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl, C₃-C₈-cycloalkyl; where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another; and
R^{8f} represents methyl and
X¹ represents nitrogen or CR³,
X² represents nitrogen or CR⁴,
A represents C(R¹⁴)₂ or a direct bond,
R¹ to R⁵ independently of one another represent hydrogen, halogen, cyano, hydroxyl, nitro, a 3- to 8-membered, unsubstituted or substituted, saturated or unsaturated cycle which may contain no or up to four heteroatoms selected from the group consisting of N, O and S, where two oxygen atoms are not adjacent, OR¹², SR¹², SOR¹², SO₂R¹², SON(R¹²)₂, SO₂N(R¹²)₂, OSO₂N(R¹²)₂, C═OR¹², NR¹²COOR¹³, NR¹²(C═S)OR¹³, N(R¹²)₂, NR¹²COR¹², NR¹²SO₂R¹³, NR¹²SOR¹³, OCON(R¹²)₂, OC═OR¹², CON(R¹²)₂, COOR¹², C(R¹²)₂OR¹², (CH₂)ₘC(R¹²)₂OR¹², (CH₂)ₘOR₁₂, (CH₂)ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, (CH₂)ₘCOOR¹², (CH₂)ₘCOR¹², (CH₂)ₘNR¹²COR¹², (CH₂)ₘNR¹²COOR¹³, unsubstituted or substituted C₁-C₈-alkyl, C₁-C₈-haloalkyl;
where m=1-8,
where additionally or independently thereof in each case two adjacent radicals R², R³ or R⁴, if appropriate via R¹² or R¹³, together may form a 3- to 7-membered, unsubstituted or substituted, saturated or unsaturated cycle which may contain no or up to four heteroatoms selected from the group consisting of N, O and S, where two oxygen atoms are not adjacent to one another,
R⁶ represent hydrogen, unsubstituted or substituted benzyl, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, C₃-C₈-halocycloalkyl)carbonyl having in each case 1 to 9 fluorine, chlorine and/or bromine atoms, or —CH₂—CH≡CH—R^{1-A}, —CH_{2—CH═CH—R}^{1-A}, —CH═C═CH—R^{1-A}, C(═O)C(═O)R², —CONR³R⁴, —CH₂NR⁵R⁶, C₁-C₆-trialkylsilyl, C₁-C₄-trialkylsilylethyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R⁷ represents hydrogen, cyano, methyl, CF₃ CFH₂ or CF₂H,
R⁸ represents fluorine, chlorine, bromine, iodine, cyano, methyl, C₁-C₃-haloalkyl,
R⁹ represents hydrogen, unsubstituted or substituted C₁-C₈-alkyl, C₃-C₈-cycloalkyl, C₁-C₈-haloalkyl, C₁-C₄-trialkylsilyl, unsubstituted or substituted aryl, unsubstituted or substituted benzyl, C₁-C₄-alkylC(═O), C₁-C₄-alkylOC(═O), unsubstituted or substituted C₁-C₄-alkoxy(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl,
R¹⁰ represents hydrogen, C₁-C₈-alkyloxy, unsubstituted or substituted C₁-C₈-alkyl, C₃-C₈-cycloalkyl, C₁-C₈-haloalkyl, C₁-C₄-trialkylsilyl, unsubstituted or substituted aryl, COOR¹², C═OR¹², OR¹²,
R¹¹ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl, unsubstituted or substituted aryl, unsubstituted or substituted cyclopropyl,
R¹² represents identical or different hydrogen, unsubstituted or substituted C₁-C₈-alkyl, unsubstituted or substituted C₁-C₈-haloalkyl, unsubstituted or substituted C₃-C₆-cycloalkyl, C₁-C₄-trialkylsilyl, unsubstituted or substituted C₂-C₆-alkenyl, unsubstituted or substituted C₃-C₆-alkynyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
or
if two radicals R¹² are attached to a nitrogen atom, two radicals R¹² may form a 3- to 7- membered, unsubstituted or substituted, saturated or unsaturated cycle which may contain up to four further heteroatoms selected from the group consisting of N, O and S, where two oxygen atoms are not adjacent to one another,
or
if two radicals R¹² are adjacent to one another in the grouping NR¹²COR¹², two radicals R¹² may form a 3- to 7-membered, unsubstituted or substituted, saturated or unsaturated cycle which may contain up to four further heteroatoms selected from the group consisting of N, O and S, where two oxygen atoms are not adjacent to one another,
R¹³ represents identical or different unsubstituted or substituted C₁-C₈-alkyl, unsubstituted or substituted C₁-C₈-haloalkyl, C₁-C₄-trialkylsilyl, unsubstituted or substituted C₁-C₆-alkenyl, unsubstituted or substituted C₁-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered, unsubstituted or substituted, saturated or unsaturated cycle which may contain no or up to four heteroatoms selected from the group consisting of N, O and S, where two oxygen atoms are not adjacent to one another,
where two radicals R¹³ may form a 3- to 7-membered, unsubstituted or substituted, saturated or unsaturated cycle which may contain up to four further heteroatoms selected from the group consisting of N, O and S, where two oxygen atoms are not adjacent to one another,
R¹⁴ represents identical or different hydrogen, fluorine, chlorine, bromine, unsubstituted or substituted C₁-C₈-alkyl, C₁-C₈-alkyloxy, C₃-C₈-cycloalkyl, C₁-C₈-haloalkyl, C₁-C₄-trialkylsilyl,
where two radicals R¹⁴ may also form a carbonyl or thiocarbonyl group (C═O or C═S), and/or an agrochemically active salt thereof.

13. Composition for controlling unwanted microorganisms comprising, at least one diaminopyrimidine selected from compounds of the formulae (Ia) according to claim 7 and an extender and/or surfactant.

14. Composition according to claim 13, further comprising at least one further agrochemically active compound.

15. A fungicide for controlling unwanted microorganisms comprising a diaminopyrimidine selected from compounds of formulae (Ib) according to claim 8.

16. Method for controlling unwanted microorganisms, comprising applying a diaminopyrimidine selected from compounds of formulae (Ic) according to claim 9 to the unwanted microorganisms and/or a habitat thereof.

17. Process for preparing compositions for controlling unwanted microorganisms, comprising mixing a diaminopyrimidine selected from compounds of formulae (Id), according to claim 10 with an extender and/or surfactant.

18. A fungicide for controlling unwanted microorganisms comprising a-diaminopyrimidine selected from compounds of formulae I(e) according to claim 11.

19. A fungicide for controlling unwanted microorganisms comprising a-diaminopyrimidine selected from compounds of formulae I(f) according to claim 12.
